(19) United States
(12) Patent Application Publication
(10) Pub. No.: US 2023/0124492 A1
(43) Pub. Date: Apr. 20, 2023

(54) COMPOSITIONS AND METHODS FOR SUBSTITUTED 7-(PIPERAZIN-1-YL)PYRAZOLO[1,5-A]PYRIMIDINE ANALOGS AS INHIBITORS OF KRAS

(71) Applicant: The Board of Regents of the University of Texas System, Austin, TX (US)

(72) Inventors: Cynthia V. Pagba, Houston, TX (US); Alemayehu Gorfe Abebe, Houston, TX (US); Scott R. Gilbertson, Houston, TX (US); Kasuni Dilsha, Houston, TX (US)

(21) Appl. No.: 17/784,078

(22) PCT Filed: Dec. 10, 2020
(86) PCT No.: PCT/US2020/064356

§ 371 Date: Jun. 9, 2022

Related U.S. Application Data
(60) Provisional application No. 62/946,138, filed on Dec. 10, 2019.

(51) Int. Cl. C07D 487/04; A61P 35/00; A61K 45/06; A61K 31/519

(52) U.S. Cl. CPC C07D 487/04; A61P 35/00; A61K 45/06; A61K 31/519

(57) ABSTRACT

In one aspect, the disclosure relates to compounds useful as inhibitors of mutant KRAS proteins, methods of making the same, pharmaceutical compositions comprising the same, and methods of treating cancers associated with mutated forms of KRAS using the same. This abstract is intended as a scanning tool for purposes of searching in the particular art and is not intended to be limiting of the present disclosure.

### CROSS-REFERENCE TO RELATED APPLICATIONS

This application claims the benefit of U.S. Provisional Appl. No. 62/946,138, fil d Dec. 10, 2019. The content of the foregoing application is relied upon and is incorporated by reference herein in its entirety.

### STATEMENT OF FEDERALLY SPONSORED RESEARCH AND DEVELOPMENT

This invention was made with government support under Grant No. GM124233 awarded by the National Institutes of Health. The government has certain rights in the invention.

### BACKGROUND

KRAS is a proto-oncogene encoding a protein known as K-Ras, which is, in turn, an element of the RAS/MAP K signaling pathway. The KRAS protein typically relays signals regarding growth and proliferation and or differentiation from outside the cell to the nucleus. KRAS-is a GTPase protein and must bind to a molecule of guanosine triphosphate (GTP) in order to transmit a signal. When bound to guanosine diphosphate (GDP), which happens a result of GTP hydrolysis, KRAS protein is inactive. KRAS is typically found bound to cell membranes due to a hydrophobic group located at its C-terminus.

A mutation affecting a single amino acid can activate KRAS decrease GTP hydrolysis activity, leading to prolonged KRAS activation and increased signal transduction leading to uncontrolled cell proliferation.

KRAS mutations in somatic cells are associated with as many as 2030% of human cancers, including 95% of pancreatic cancers and 45% of colorectal cancers, as well as ovarian, gastric, uterine, and lung cancers. Mutations G12V, G12D, G13D, and Q61H are some of the most common and most deleterious activating mutations. Thus, KRAS would be a highly-desirable drug target. However, efforts to target KRAS with small molecules have thus far proven to be largely unsuccessful. GTP and GDP have high affinities (picomolar) for the GTP/GDP binding site, rendering it inaccessible to small molecule inhibitors. KRAS lacks other obvious binding sites for small molecules and attempts at indirect inhibition, such as interfering with the functionally required targeting of KRAS to the plasma membrane, have also failed thus far.

Despite advances in KRAS inhibition research, there is still a scarcity of compounds that are potent, efficacious, and selective inhibitors of KRAS that are effective in the treatment of cancers associated with KRAS activation. A large gap remains between laboratory data on KRAS and clinically relevant outcomes. These needs and other, needs are satisfied by the present disclosure.

### SUMMARY

In accordance with the purpose(s) of the invention, as embodied and broadly described herein, the invention, in one aspect, relates to compounds useful as inhibitors of mutant KRAS proteins, methods of making the same, pharmaceutical compositions comprising the same, and methods of treating cancers associated with mutated forms of KRAS using the same.

Disclosed are compounds having a structure represented by a formula:

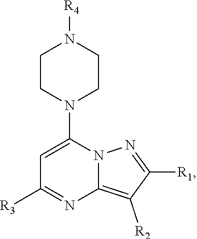

wherein R₁ is aryl, substituted aryl, —(C1-C7 alkanediyl)-R₅, or —(C01-C7 alkanediyl)-(C═O)—R₅; wherein each of R₂ and R₃ is independently C1-C8 alkyl, cycloalkyl, heterocycloalkyl, aryl, heteroaryl, substituted aryl, or substituted heteroaryl; wherein R₄ is —(C1-C7 alkyl) or —(C1-C7 alkanediyl)-R₆; and wherein each of R₅ and R₆ is independently hydrogen, methoxy, —SF₅, —OH, —SH, —NH₂ or halogen; or a pharmaceutically acceptable salt, solvate, or ester thereof.

Also disclosed are pharmaceutical compositions comprising a therapeutically effective amount of one or more disclosed compounds, or pharmaceutically acceptable salt thereof, and a pharmaceutically acceptable carrier.

Also disclosed are methods for the treatment of a cancer associated with KRAS dysfunction in a mammal comprising the step of administering to the mammal a therapeutically effective amount of at least one disclosed compound or pharmaceutically acceptable salt thereof.

Also disclosed are methods for reducing or eliminating of KRAS signal transduction activity in a mammal comprising the step of administering to the mammal a therapeutically effective amount of at least one disclosed compound or pharmaceutically acceptable salt thereof.

Also disclosed are methods for reducing or eliminating of KRAS signal transduction activity in at least one cell, comprising the step of contacting the cell with an effective amount of at least one disclosed compound or pharmaceutically acceptable salt thereof.

Also disclosed are uses of a disclosed compound, or a pharmaceutically acceptable salt thereof; a disclosed product of making, or a pharmaceutically acceptable salt thereof; or a disclosed pharmaceutical composition.

Also disclosed are uses of a disclosed compound, or a pharmaceutically acceptable salt thereof, in the manufacture of a medicament for the treatment of a disorder, associated with KRAS signal transduction dysfunction in a mammal.

Also disclosed are methods for the manufacture of a medicament to inactivate or inhibit KRAS signal transduction in a mammal comprising combining at least one disclosed compound, or a pharmaceutically acceptable salt thereof with a pharmaceutically acceptable carrier or diluent.

Also disclosed are kits comprising at least one disclosed compound, or a pharmaceutically acceptable salt, hydrate, solvate, or ester thereof, and one or more of: a) at least one agent known to decrease KRAS activity; (b) at least one agent known to treat a disorder associated with increased KRAS activity; (c) instructions for treating a disorder associated with increased KRAS activity; and/or (d) instructions for administering the compound in connection with radiation therapy or another anti-cancer therapy.

While aspects of the present disclosure can be described and claimed in a particular statutory class, such as the system statutory class, this is for convenience only and one of skill in the art will understand that each aspect of the present disclosure can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

### BRIEF DESCRIPTION OF THE DRAWINGS

The accompanying figures, which are incorporated in and constitute a part of this specification, illustrate several aspects and together with the description serve, to explain the principles of the disclosure.

FIGS. 1A-H show microscale thermophoresis data the binding of representative compounds (pictured) to KRAS.

FIGS. 2A-I illustrates the measurements of binding affinity of ligands to wild type (WT) or G120 KRAS bound to the GTP analogue GNP and, for selected exemplary embodiments, effect of ligands on the rate of SOS-mediated nucleotide exchange reaction. Binding affinity was determined using intrinsic fluorescence, which made use of the ligands' intrinsic fluorescence and the accompanying change (decrease or increase in intensity) upon binding to KRAS.

Additional advantages of the disclosure will be set forth in part in the description which follows, and in part will be obvious from the description, or can be learned by practice of the disclosure. The advantages of the disclosure will be realized and attained by means of the elements and combinations particularly pointed out in the appended claims. It is to be understood that both the foregoing general description and the following detailed description are exemplary and explanatory only and are not restrictive of the disclosure, as claimed.

### DETAILED DESCRIPTION

Many modifications and other embodiments disclosed herein will come to mind to one skilled in the art to which the disclosed compositions and methods pertain having the benefit of the teachings presented in the foregoing descriptions and the associated drawings, Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications and patents cited in this specification are cited to disclose and describe the methods and/or materials in connection with which the publications are cited. All such publications and patents are herein incorporated by references as if each individual publication or patent were specifically and individually indicated to be incorporated by reference. Such incorporation by reference is expressly limited to the methods and/or materials described in the cited publications and patents and does not extend to any lexicographical definitions from the cited publications and patents. Any lexicographical definition in the publications and patents cited that is not also expressly repeated in the instant application should not be treated as such and should not be read as defining any terms appearing in the accompanying claims. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

While aspects of the present disclosure can be described and claimed in a particular statutory class, such as the system statutory class, this is for convenience only and one of skill in the art will understand that each aspect of the present disclosure can be described and claimed in any statutory class.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Aspects, of the present disclosure will employ, unless otherwise indicated, techniques of molecular biology, microbiology, organic chemistry, biochemistry, physiology, cell biology, blood vessel biology, and the like, which are within the skill of the art. Such techniques are explained fully in the literature.

Prior to describing, the various aspects of the present disclosure, the following definitions are provided and should be used unless otherwise indicated. Additional terms may be defined elsewhere in the present disclosure.

### Definitions

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of” Similarly, the term “consisting essentially of” is intended to include examples encompassed by the term “consisting of”.

As used herein, the term “and/or” includes any and all combinations of one or more of the associated listed items, Expressions such as “at least one of,” when preceding a list of elements, modify the entire list of elements and do not modify the individual elements of the list.

As used in the specification and the appended claims, the singular forms “a,” “an” and “the” include plural referents unless the context clearly dictates otherwise. Thus, for example, reference to “a KRAS inhibitor” or “a cancer” include, but are not limited to, two or more such KRAS inhibitors or cancers, and the like.

Reference to “a/an” chemical compound refers to one or more molecules of the chemical compound rather than being limited to a single molecule of the chemical compound. Furthermore, the one or more molecules may or may not be identical, so long as they fall under the category of the chemical compound. Thus, for example, “a” KRAS inhibitor is interpreted to include one or more small molecule inhibitors of KRAS, where the small molecules may or may not be identical (e.g., different pendant groups may be located on the pyrazolopyrimidine ring system disclosed herein as can be found in a multidrug cocktail).

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent about,” it will be understood that the particular value forms a further aspect. For example, if the value “about 10” is disclosed, then “10” is also disclosed.

Where a range is expressed, a further aspect includes from the one particular value and/or to the other particular value. Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, ‘less than y’, and ‘less than z’. Likewise, the phrase ‘about x, y, z, or greater’ should be interpreted to include the specific ranges of ‘about x’, ‘about y’, and ‘about z’ as well as the ranges of ‘greater than x’, ‘greater than y’, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3 to, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used, herein, “about,” “approximately,” substantially, and the like, when used in connection with a numerical variable, can generally refers to the value of the variable and to all values of the variable that are within the experimental error (e.g., within the 95% confidence interval for the mean) or within +/−10% of the indicated value, whichever is greater. As used herein, the terms “about,” “approximate,” “at or about,” and “substantially” can mean that the amount or value in question can be the exact value or a value that provides equivalent results or, effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, the terms “optional” “optionally” means that the subsequently described event or circumstance can or cannot occur, and that the description includes instances where said event or circumstance occurs and instances where it does not.

As used herein, “EC₅₀,” is intended to refer to the concentration of a substance (e.g., a compound or a drug) that is required for 50% activation or enhancement of a biological process, or component of a process. For example, EC₅₀ can refer to the concentration of KRAS inhibitor that provokes a response halfway between the baseline and maximum response in an appropriate assay of the target activity. For example, an EC₅₀ for a KRAS inhibitor can be determined in an in vitro or cell-based assay system. Such in vitro assay systems frequently utilize a cell line that either expresses endogenously a target of interest, or has been transfected with a suitable expression vector that directs expression of a recombinant form of the target such as a mutant form of KRAS.

As used herein, “IC 50” is intended to refer to the concentration of a substance (e.g., a compound or a drug) that is required for 50% inhibition of a biological process, or component of a process. For example, IC₅₀ refers to the half maximal (50%) inhibitory concentration (IC) of a substance as determined in a suitable assay. For example, an IC₅₀ for KRAS can be determined in an in vitro or cell-based assay system. Frequently, receptor assays, including suitable assays for KRAS inhibition, make use of a suitable cell-line, e.g. a cell line that either expresses endogenously a target of interest, or has been transfected with a suitable expression vector that directs expression of a recombinant form of the target such as mutant, activated KRAS.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valences of the heteroatoms. This disclosure is not intended to be limited in, any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc. It is also contemplated that, in certain aspects, unless expressly indicated to the contrary, individual substituents can be further optionally substituted (i.e., further substituted or unsubstituted).

In defining various terms, “A₁,” “A₂,” “A₃” and “A₄” are used herein as generic symbols to represent various specific substituents. These symbols can be any substituent, not limited to those disclosed herein, and when they are defined to be certain substituents in one instance, they can, in another instance, be defined as some other substituents.

The term “aliphatic” or “aliphatic group,” as used herein, denotes a hydrocarbon moiety that may be straight-chain (i.e., unbranched), branched, or cyclic (including fused, bridging, and spirofused polycyclic) and may be completely saturated or may contain one or more units of unsaturation, but which is not aromatic. Unless otherwise specified, aliphatic groups contain 1-20 carbon atoms. Aliphatic groups include, but are not limited to, linear or branched, alkyl, alkenyl, and alkynyl groups, and hybrids thereof such as (cycloalkyl)alkyl, (cycloalkenyl)alkyl or (cycloalkyl)alkenyl.

The term “alkyl” as used herein is a branched or unbranched saturated hydrocarbon group oft to 24 carbon atoms, such as methyl, ethyl, n-propyl, isopropyl, n-butyl, isobutyl, s-butyl, t-butyl, n-pentyl, isopentyl, s-pentyl, neopentyl, hexyl, heptyl, octyl, nonyl, decyl, dodecyl, tetradecyl, hexadecyl, eicosyl, tetracosyl, and the like. The alkyl group can be cyclic or acyclic. The alkyl group can be branched or unbranched. The alkyl group can also be substituted or unsubstituted. For example, the alkyl group can be substituted with one or more groups including, but not limited to, alkyl, cycloalkyl, alkoxy, amino, ether, halide, hydroxy, nitro, silyl, sulfo-oxo, or thiol, as described herein. A “lower alkyl” group is an alkyl group containing from one to six (e.g., from one to four) carbon atoms. The term alkyl group, can also be a C1 alkyl, C1-C2 alkyl, C1-C3 alkyl, C1-C4 alkyl, C1-05 alkyl, C1-C6 alkyl, C1-C7 alkyl, C1-C3 alkyl, C1-C9 alkyl, C1-C10 alkyl, and the like up to and including a C1-C24 alkyl.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” or “haloalkyl” specifically refers to an alkyl group that is substituted with one or more halide, e.g., fluorine, chlorine, bromine, or iodine. Alternatively, the term “monohaloalkyl” specifically refers to an alkyl group that is substituted with a single halide, e.g. fluorine, chlorine, bromine, or iodine. The term “polyhaloalkyl” specifically refers to an alkyl group that is independently substituted with two or more halides, i.e. each halide substituent need not be the same halide as another halide substituent, nor do the multiple instances of a halide substituent need to be on the same carbon. The term “alkoxyalkyl” specifically refers to an alkyl group that is substituted with one or more alkoxy groups, as described below. The term “aminoalkyl” specifically refers to an alkyl group that is substituted with one or more amino groups. The term “hydroxyalkyl” specifically refers to an alkyl group that is substituted with one or more hydroxy groups. When “alkyl” is used in one instance and a specific term such as “hydroxyalkyl” is used in another, it is not meant to imply that the term “alkyl” does not also refer to specific terms such as “hydroxyalkyl” and the like.

This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl, can be referred to as, e.g., an “alkylcycloalkyl.” Similarly, a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl” and a specific term, such as “alkylcycloalkyl” is not meant to imply that the general term does not also include the specific term.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, norbomyl, and the like. The term “heterocycloalkyl” is a type of cycloalkyl group as defined above, and is included within the meaning of the term “cycloalkyl” where at least one of the carbon atoms of the ring is replaced with a heteroatom such as, but not limited to, nitrogen, oxygen, sulfur or phosphorus. The cycloalkyl group and heterocycloalkyl group can be substituted or unsubstituted. The cycloalkyl group and heterocycloalkyl group can be substituted with one or more groups including, but not limited to, alkyl, cycloalkyl, alkoxy, amino, ether halide, hydroxy, nitro, silyl, sulfa-oxo, or thiol as described herein.

The term “alkanediyl” as used herein, refers to a divalent saturated aliphatic group, with one or two saturated carbon atom(s) as the point(s) of attachment, a linear or branched, cyclo, cyclic or acyclic structure, no carbon-carbon double or triple bonds, and no atoms other than carbon and hydrogen. The groups, —CH₂-(methylene), —CH₂CH₂—, —CH₂C(CH₃)₂CH₂—, and —CH₂CH₂CH₂—are non-limiting examples of alkanediyl groups.

The terms “alkoxy” and “alkoxyl” as used herein to refer to an alkyl or cycloalkyl group bonded through an ether linkage; that, is, an “alkoxy” group can be defined as—OA₁ where A₁ is alkyl or cycloalkyl as defined above. “Alkoxy” also includes polymers of alkoxy groups as just described; that is, an alkoxy can be a polyether such as—OA₁—OA₂ or—OA₁—(OA₂)ₐ—OA₃, where “a” is an integer of from 1 to 200 and A₁, A_{z}, and A₃ are alkyl and/or cycloalkyl groups.

The term “alkenyl” as used herein is a hydrocarbon group of from 2 to 24 carbon atoms with a structural formula containing at least one carbon-carbon double bond. Asymmetric structures such as (A₁A₂)C═C(A₃A₄) are intended to include both the E and Z isomers. This can be presumed in structural formulae herein wherein an asymmetric alkene is present, or it can be explicitly indicated by the bond symbol C═C. The alkenyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

The term “cycloalkenyl” as used herein is a non-aromatic carbon-based ring composed of at least three carbon atoms and containing at least one carbon-carbon double bound, i.e., C═C. Examples of cycloalkenyl groups include, but are not limited to, cyclopropenyl, cyclobutenyl, cyclopentenyl, cyclopentadienyl, cyclohexenyl, cyclohexadienyl, norbornenyl, and the like. The term “heterocycloalkenyl” is a type of cycloalkenyl group as defined above, and is included within the meaning of the term “cycloalkenyl,” where at least one of the carbon atoms of the ring is replaced with a heteroatom such as, but not limited to, nitrogen, oxygen, sulfur, or phosphorus. The cycloalkenyl group and heterocycloalkenyl group can be substituted or unsubstituted. The cycloalkenyl group and heterocycloalkenyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfa-oxo, or thiol as described herein.

The term “alkynyl” as used herein is a hydrocarbon group of 2 to 24 carbon atoms with a structural formula containing at, least one carbon-carbon triple bond. The alkynyl group can be unsubstituted or substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfur-oxo, or thiol, as described herein.

The term “cycloalkynyl” as used herein is a non-aromatic carbon-based ring composed of at least seven carbon atoms and containing at least one carbon-carbon triple bound. Examples of cycloalkynyl groups include, but are not limited to, cycloheptynyl, cyclooctynyl, cyclononynyl, and the like. The term “heterocycloalkynyl” is a type of cycloalkenyi group as defined above, and is included within the meaning of the term “cycloalkynyl,” where at least one of the carbon atoms of the ring is replaced with a heteroatom such as, but not limited to, nitrogen, oxygen, sulfur, or phosphorus. The cycloalkynyl group and heterocycloalkynyl group can be substituted or unsubstituted. The cycloalkynyl group and heterocycloalkynyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo or thiol as described herein.

The term “aromatic group” as used herein refers to a ring structure having, cyclic clouds of delocalized TT electrons above and below the plane of the molecule, where the TT clouds contain (4n+2) TT electrons, A further discussion of aromaticity is found in Morrison and Boyd, Organic Chemistry, (5th Ed., 1987), Chapter 13, entitled “Aromaticity,” pages 477-497, incorporated herein by reference. The term “aromatic group” is inclusive of both aryl and heteroaryl groups.

The term “aryl” as used herein is a group that contains any carbon-based aromatic group including, but not limited to, benzene, naphthalene, phenyl, biphenyl, anthracene, and the like. The aryl group can be substituted or unsubstituted. The aryl group can be substituted with one or more groups including, but not limited to, alkyl, cycloalkyl, alkoxy, alkenyl, cycloalkenyi, alkynyl, cycloalkynyl, aryl, heteroaryl, aldehyde, —NH₂, carboxylic acid, ester, ether, halide, hydroxy, ketone, azide, nitro, silyl, sulfo-oxo, or thiol as described herein. The term “biaryl” is a specific type of aryl group and is included in the definition of “aryl.” In addition, the aryl group can be a single ring structure or comprise multiple ring structures that are either fused ring structures or attached via one or ore bridging groups such as a carbon-carbon bond. For example, biaryl to two aryl groups that are bound together via a fused ring structure, as in naphthalene, or are attached via one or more carbon-carbon bonds, as in biphenyl.

The term “aldehyde” as used herein is represented by the formula —C(O)H. Throughout this specification “C(P)” is a short hand notation for a carbonyl group, i.e., C═O.

The terms “amine” or “amino” as used herein are represented by the formula—NA₁A₂, where A₁ and A₂ can be, independently, hydrogen or alkyl, cycloalkyl, alkenyl, cycloalkenyi, alkynyl, cycloalkynyl, aryl, or heteroaryl group as described herein, A specific example of amino is —NH₂.

The term “alkylamino” as used herein is represented by the formula —NH₁(-alkyl) and —N(-alkyl)₂, where alkyl is a described herein. Representative examples include, but are not limited to, methylamino group, ethylarnino group, propylamino group, isopropylamino group, butylamino group, isobutylamino group, (sec-butyl)amino group, (tert-butyl)amino group, pentyiamino group, isopentylamino group, (tert-pentyl)amino group, hexylamino group, climethylamino group, diethylamino group, dipropylamino group, diisopropylamino group, dibutylamino group, diisobutylamino group, di(sec-butyl)amino group, di(tert-butyl)amino group, dipentylamino group, diisopentylamino group, di(tert-pentyl)amino group, dihexylamino group, N-ethyl-N-methylarnino group, N-methyl-N-propylamino group, N-ethyl-N-propylamino group and the like.

The term “carboxylic acid” as used herein is represented by the formula —C(O)OH.

The term “ester” as used herein is represented by the formula —OC(O)A₁ or C(O)OA₁, where A₁ can be alkyl, cycloalkyl, alkenyl, cycloalkenyl, alkynyl, cycloalkynyl, aryl, or heteroaryl group as described herein. The term “polyester” as used herein is represented by the formula—(A₁O(O)C—A₂—C(O)O)ₐ— or—(A₁O(O)C—A₂—OC(O))ₐ—, where A₁ and A₂ can be, independently, an alkyl, cycloalkyl, alkenyl, cycloalkenyl, alkynyl, cycloalkynyl, aryl, or heteroaryl group described herein and “a” is an integer from 1 to 500. “Polyester” is as the term used to describe a group that is produced by the reaction between a compound having at least two carboxylic acid groups with a compound having at least two hydroxyl groups.

The term “ether” as used herein is represented by the formula A₁OA₂, where A₁ and A₂ can be, independently, an alkyl, cycloalkyl, alkenyl, cycloalkenyl, alkynyl, cycloalkynyl, aryl, or heteroaryl group described herein. The term “polyether” as used herein is represented by the formula—(A₁O-A₂O)ₐ—, where A₁ and A₂ can be, independently, an alkyl, cycloalkyl, alkenyl, cycloalkenyl, alkynyl, cycloalkynyl, aryl, or heteroaryl group described herein and “a” is an integer of from 1 to 500. Examples of polyether groups include polyethylene oxide, polypropylene oxide, and polybutylene oxide.

The terms “halo,” halogen or “halide,” as used herein can be used interchangeably and refer to F, Cl, Br, or I.

The terms “pseudohalide,” “pseudohalogen” or “pseudohalo,” as used herein can be used interchangeably and refer to functional groups that behave substantially similar to halides. Such functional groups include, by way of example, cyano, thiocyanato, azido, trifluoromethyl, trifluoromethoxy, perfluoroalkyl and perfluoroalkoxy groups.

The term “heteroalkyl” as used herein refers to an alkyl group containing at least one heteroatom. Suitable heteroatoms include, but are not limited to, O, N, Si, P and S, wherein the nitrogen, phosphorous and sulfur atoms are optionally oxidized, and the nitrogen heteroatom is optionally quaternized. Heteroalkyls can be substituted as defined above for alkyl groups.

The term “heteroaryl” as used herein refers to an aromatic group that has at least one heteroatom incorporated within the ring of the aromatic group. Examples of heteroatoms include, but are not limited, to, nitrogen, oxygen, sulfur, and phosphorus, where N-oxides, sulfur oxides, and dioxides are permissible heteroatom substitutions. The heteroaryl group can be substituted or unsubstituted. The heteroaryl group can be substituted with one or more groups including, but not limited to, alkyl, cycloalkyl, alkoxy, amino, ether, halide, hydroxy, nitro, silyl, sulfo-oxo, or thiol as described herein. Heteroaryl groups can be monocyclic, or alternatively fused ring systems. Heteroaryl groups include, but are not limited to, furyl, imidazolyl, pyrimidinyl, tetrazolyl, thienyl, pyridinyl, pyrolyl, N-methylpyrrolyl, quinolinyl, isoquinolinyl, pyrazolyl, triazolyl, thiazolyl, ox tolyl, isoxazolyl, oxadiazolyl, thiadiazolyl, isothiazolyl, pyridazinyl, pyrazinyl, benzofuranyl, benzodioxolyl, benzothiophenyl, indolyl, indazolyl, benzimidazolyl, imidazopyridinyl, pyrazolopyridinyl, and pyrazolopyrimidinyl. Further not limiting examples of heteroaryl groups include, but are not limited to, pyridinyl, pyridazinyl, pyrimidinyl, pyrazinyl, thiophenyl, pyrazolyl, imidazolyl, benzo[d]oxazoyl, benzo[d]thiazolyl, quinolinyl, quinazolinyl, indazolyl, imidazo[1,2-b]pyridazinyl, imidazo[1,2-a]pyrazinyl, benzo[c][1,2,5]thiadiazoyl, benzo[c][1,2,5]oxadiazolyl, and pyrido[2,3-b]pyrazinyl.

The terms “heterocycle” or “heterocyclyl,” as used herein can be used interchangeably and refer to single and multi-cyclic aromatic or non-aromatic ring systems in which at least one of the ring members is other than carbon. Thus, the term is inclusive of, but not limited to, “heterocycloalkyl,” “heteroaryl,” “bicyclic heterocycle,” and “polycyclic heterocycle.” Heterocycle includes pyridine, pyrimidine, furan, thiophene, pyrrole, isoxazole, isothiazole, pyrazole, oxazole: thiazole, imidazole, oxazole, including, 1,2,3-oxadiazole, 1,2,5-oxadiazole and 1,3,4-oxadiazole, thiadiazole, including, 1,2,3-thiadiazole, 1,2,5-thiadiazole, and 1,3,4-thiadiazole, triazole, including, 1,2,3-triazole, 1,3,4-triazole, tetrazole, including 1,2,3,4-tetrazole and 1,2,4,5-tetrazole, pyridazine, pyrazine, triazine, including 1,2,4-triazine and 1,3,5-triazine, tetrazine, including 1,2,4,5-tetrazine, pyrrolidine, piperidine, piperazine, morpholine, azetidine, tetrahydropyran, tetrahydrofuran, dioxane, and the like. The term heterocyclyl group can also be a C2 heterocyclyl, C2-C3 heterocyclyl, C2-C4 heterocyclyl, C2-C5 heterocyclyl, C2-C6 heterocyclyl, C2-C7 heterocyclyl, C2-C8 heterocyclyl, C2-C9 heterocyclyl, C2-C10 heterocyclyl, C2-C11 heterocyclyl, and the like up to and including a C2-C18 heterocyclyl. For example, a C2 heterocyclyl comprises a group which has two carbon atoms and at least one heteroatom, including, but not limited to, aziridinyl, diazetidinyl, dihydrodiazetyl: oxiranyl, thiiranyl, and the like. Alternatively, for example, a C5 heterocyclyl comprises a group which has five carbon atoms and at least one heteroatom, including, but not limited to, piperidinyl, tetrahydropyranyl, tetrahydrothiopyranyl, diazepanyl, pyridinyl, and the like. It is understood that a heterocyclyl group may be bound either through a heteroatom in the ring, where chemically possible, or one of carbons comprising the heterocyclyl ring.

The term “bicyclic heterocycle” or “bicyclic heterocyclyl” as used herein refers to a ring system in which at least one of the ring members is other than carbon. Bicyclic heterocyclyl encompasses ring systems wherein an aromatic ring is fused with another aromatic ring, or wherein an aromatic ring is fused with a non-aromatic ring. Bicyclic heterocyclyl encompasses ring systems wherein a benzene ring is fused to a 5- or a 6-membered ring containing 1, 2 or 3 ring heteroatoms or wherein a pyridine ring is fused to a 5- or a 6-membered ring containing 1, 2 or 3 ring heteroatoms, Bicyclic heterocyclic groups include, but are not limited to, indolyl, indazolyl, pyrazolo[1,5-a]pyridinyl, benzofuranyl, quinolinyl, quinoxalinyl, 1,3-benzodioxolyl, 2,3-dihydro-1,4-benzodioxinyl, 3,4-dihydro-2H-chromenyl, 1H-pyrazolo[4,3-c]pyridin-3-yl; 1H-pyrrolo[3,2-b]pyridin-3-yl; and 1H-pyrazolo[3,2-b]pyridin-3-yl.

The term “heterocycloalkyl” as used herein refers to an aliphatic, partially unsaturated or fully saturated, 3- to 14-membered ring system, including single rings of 3 to 8 atoms and bi- and tricyclic ring systems. The heterocycloalkyl ring-systems include one to four heteroatoms independently selected from oxygen, nitrogen, and sulfur, wherein a nitrogen and sulfur heteroatom optionally can be oxidized and a nitrogen heteroatom optionally can be substituted. Representative heterocycloalkyl groups include, but are not limited to, pyrrolidinyl, pyrazolirtyl, pyrazolidinyl, imidazolinyl, imidazolidinyl, piperidinyl, piperazinyl, oxazolidinyl, isoxazolidinyl, morpholinyl, thiazolidinyl, isothiazolidinyl, and tetrahydrofuryl.

The term “hydroxyl” or “hydroxy” as used herein is represented by the formula —OH.

The term ketone as used herein is represented by the formula A₁C(O)A₂, where A₁ and A₂ can be, independently, an alkyl, cycloalkyl, alkenyl, cycloalkenyl, alkynyl, cycloalkynyl, aryl, or heteroaryl group as described herein.

The term “azide” or “azido” as used herein is represented by the formula —N₂.

The term “nitro” as used herein is, represented by the formula —NO₂.

The term “nitrile” or “cyano” as used herein is represented by the formula —CN.

The term “silyl” as used herein is represented by the formula —SiA₁A₂A₃, where A₁, A₂, and A₃ can be, independently, hydrogen or an alkyl, cycloalkyl, alkoxy, alkenyl, cycloalkenyl, alkynyl, cycloalkynyl, aryl, or heteroaryl group as described herein.

The term “sulfo-oxo” as used herein is represented by the formulas —S(O)A₁, —S(O)₂A₁, OS(O)₂A₁, or —OS(O)₂A₁, where A₁ can be hydrogen or an alkyl, cycloalkyl, alkenyl, cycloalkenyl, alkynyl, cycloalkynyl, aryl, or heteroaryl group as described herein. Throughout this specification “S(O)” is a short hand notation for S═O. The term “sulfonyl” is used herein to refer to the sulfo-oxo group represented by the formula —S(O)₂A₁, where A can be hydrogen or an alkyl, cycloalkyl, alkenyl, cycloalkenyl, alkynyl, cycloalkynyl, aryl, or heteroaryl group as described herein. The term “sulfone” as used herein is represented by the formula A₁S(O)₂A₂, where A₁ and A₂ can be, independently, an alkyl, cycloalkyl, alkenyl, cycloalkenyl, alkynyl, cycloalkynyl, aryl, or heteroaryl group as described herein. The term “sulfoxide” as used herein is represented by the formula A₁S(O)A₂, where A₁ and A₂ can be, independently, an alkyl, cycloalkyl, alkenyl, cycloalkenyl, alkynyl, cycloalkynyl, aryl, or heteroaryl group as described herein.

The term “thiol” as used herein is represented by the formula —SH.

“R₁,” “R₂,” “R₃,” . . . “Rₙ,” where n is an integer, as used herein can, independently, possess one or more of the groups listed above. For example, if R₁ is a straight chain alkyl group, one of the hydrogen atoms of the alkyl group can optionally be substituted with a hydroxyl group, an alkoxy group, an alkyl group, a halide, and the like. Depending upon the groups that are selected, a first group can be incorporated within second group or, alternatively, the first group can be pendant (i.e., attached) to the second group. For example, with the phrase “an alkyl group comprising an amino group,” the amino group can be incorporated within the backbone of the alkyl group. Alternatively, the amino group can be attached to the backbone of the alkyl group. The nature of the group(s) that is (are) selected will determine if the first group is embedded or attached to the second group.

As described herein, compounds of the invention may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. In is also contemplated that, in certain aspects, unless expressly indicated to the contrary, individual substituents can be further optionally substituted (i.e., further substituted or unsubstituted).

The term “stable,” as used herein, refers to compounds that are not substantially altered when subjected to conditions to allow for their production, detection, and, in certain aspects, their recovery, purification, and use for one or more of the purposes disclosed herein.

Suitable monovalent substituents on a substitutable carbon atom of an “optionally substituted” group are independently halogen; —(CH2)₀₋₄R^{o}; —(CH2)₀₋₄ OR^{o}; —O(CH2)₀₋₄R^{o}, —O—(CH2)₀₋₄ C(O)OR^{o}; —(CH2)₀₋₄CH(OR^{o})₂; —(CH2)₀₋₄SR^{o}; —(CH2)₀₋₄Ph, which may be substituted with R^{o}; —(CH2)₀₋₄O(CH2)₀₋₄Ph which may be substituted with R^{o}; —CH═CHPh, which may be substituted with R^{o}; —(CH2)₀₋₄O(CH2)₀₋₁-pyridyl which may be substituted with R^{o}; —NO₂—CN; —N₃; (CH₂)₀₋₄N(R^{o})₂; —(CH2)₀₋₄N(R^{o})C(O)R^{o}; —N(R^{o})C(S)R^{o}; —(CH₂)₀₋₄N(R^{o})C(O)NR^{o}₂; N(R^{o})C(S)NR^{o}₂; —(CH₂)₀₋₄N(R^{o})C(O)OR^{o}; —N(R^{o}); —N(R^{o})C(O)R^{o}; N(R^{o})N(R^{o})C(O)NR^{o}₂; N(R^{o})N(R^{o})C(O)OR^{o}; —(CH₂)₀₋₄C(O)R^{o}; —C(S)R^{o}; —(CH₂)₀₋₄C(O)OR^{o}; —(CH₂)₀₋₄C(O)SR^{o}; (CH₂)₀₋₄C(O)OSiR^{o}₃; —(CH2)₀₋₄OC(O)R^{o}—OC(O)(CH₂)₀₋₄ SR—, SC(S)SR^{o}; —(CH₂)₀₋₄SC(O)R^{o}; —(CH₂)₀₋₄C(O)NR^{o}₂; —C(S)NR^{o}₂; —C(S)SR^{o}; (CH₂)₀₋₄OC(O)NR^{o}₂; C(O)N(OR^{o})R^{o}; —C(O)C(O)R^{o}; —C(O)CH₂C(O)R^{o}; —C(NOR^{o})R^{o}; (CH₂)₀₋₄SSR^{o}; —(CH₂)₀₋₄S(O)₂R^{o}; —(CH₂)₀₋₄S(O)₂OR^{o}; —(CH₂)₀₋₄OS(O)₂R^{o}; —S(O)₂NR^{o}₂; (CH₂)₀₋₄S(O)R^{o}; N(R^{o})S(O)₂NR^{o}₂: —N(R^{o})S(O)₂R^{o}; —N(OR^{o})R^{o}; —C(NH)NR^{o}₂; —P(O)₂R^{o}; P(O)₂R^{o}; OP(O)R^{o}₂; —OP(O)(OR^{o})₂; SiR^{o}₃: —(C₁₋₄ straight or branched alkylene)O—N(R^{o}₂; or —(C₁₋₄ straight or branched alkylene)C(O)O—N(R^{o})₂, wherein each R^{o} may be substituted as defined below and is independently hydrogen, C₁₋₆; aliphatic, —CH₂Ph, —O(CH₂)₀₋₁Ph, —CH₂—(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{o}, taken together with their intervening atoms), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{o} (or the ring formed by taking two independent occurrences of R^{o} together with their intervening atoms), are independently halogen, —(CH₂)₀₋₂R^{o}, -(haloR^{o}), —(CH₂)₀₋₂OH, —(CH₂)₀₋₂OR^{o}, —(CH(OR^{o})₂; O(haloR^{o}), —(CN), —N₃, —(CH₂)₀₋₂C(O)R^{o}, —(CH₂)₀₋₂C(O)OH, —(CH₂)₀₋₂C(O)OR^{o}, —(CH₂)₀₋₂ SR^{o}, —(CH₂)₀₋₂SH, —(CH₂)₀₋₂NH₂, —(CH₂)₀₋₂, NHR^{o}, —(CH₂)₀₋₂, NR^{o}₂, —NO₂. —SiR^{o}₃, —OSiR^{o}₃, C(O)SR^{o}, —(C₁₋₄ straight or branched alkylene)C(O)OR^{o}, or —SSW wherein each R^{o} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated, carbon atom of R^{o} include═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R*include halogen, —R^{o}, (haloR^{o}), OH, —OR^{o}, —O(haloR^{o}), —CN, —C(O)OH, —C(O)OR^{o}, —NH₂, —NHR^{o}, —NR^{o}₂, or —NO₂, wherein each R^{o} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)O(O)R^{†}, —C(O)CH₂C(O)R^{†}, —S(O)₂R^{†}, S(O)₂NR^{†}₂, —C(S)NR^{†}₂, —C(NH)NR^{†}₂, or —N(R^{†})S(O)₂R^{†}; wherein each R^{†}is independently hydrogen, C₁₋₆ aliphatic which may be substituted as defined below, unsubstituted—OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R† are independently halogen, —R^{o}, (haloR^{o}), —OH, —OR^{o}, —O(haloR^{o}), —CN, —C(O)OH, —C(O)OR^{o}, —NH₂, —NHR^{o}, —NR^{o}₂, or —NO₂, wherein each R^{o} is unsubstituted or There preceded by “halo” is substituted only with one or more halogens, and is independently C₁₋₄ aliphatic, —CH₂Pb, —O(CH₂)₀₋₁Ph, or a 5- 6-membered saturated, partially unsaturated, or aryl ring having 0-4 heteroatoms independently selected from nitrogen, oxygen, or sulfur.

The term “leaving group” refers to an atom (or a group of atoms) with electron withdrawing ability that can be displaced as a stable species, taking with, it the bonding electrons. Examples of suitable leaving groups include halides and sulfonate esters, including, but not limited to, triflate, mesylate, tosylate, and brosylate.

The terms “hydrolysable group” and “hydrolysable moiety” refer to a functional group capable of undergoing hydrolysis, e.g., under basic or acidic conditions. Examples of hydrolysable residues include, without limitation, acid halides, activated carboxylic acids, and various protecting groups known in the art (see, for example, “Protective Groups in Organic Synthesis,” T. W. Greene, P. G. M. Wuts, Wiley-interscience 1999).

A residue of a chemical species, as used in the specification and concluding claims, refers to the moiety that is the resulting product of the chemical species in a particular reaction scheme, or subsequent formulation or chemical product, regardless of whether the moiety is actually obtained from the chemical species, Thus, an ethylene glycol residue in a polyester refers to one or more—OCH₂CH₂O—units in the polyester, regardless of whether ethylene glycol was used to prepare the polyester. Similarly, a sebacic acid residue in a polyester refers to one or more—CO(CH₂)₈CO—moieties in the polyester, regardless of whether the residue is obtained by reacting sebacic acid or an ester thereof to obtain the polyester.

The term “organic residue” defines a carbon containing residue, i.e., a residue comprising at least one carbon atom, and includes but is not limited to the carbon containing groups, residues, or radicals defined hereinabove. Organic residues can contain various heteroatoms, or be bonded to another molecule through a heteroatom, including oxygen, nitrogen, sulfur, phosphorus, or the like. Examples of organic residues include but are not limited alkyl or substituted alkyls, alkoxy or substituted alkoxy, mono or di-substituted amino, amide groups, etc. Organic residues can preferably comprise 1 to 18 carbon atoms, 1 to 15, carbon atoms, 1 to 12 carbon atoms, 1 to 8 carbon atoms, 1 to 6 carbon atoms, or 1 to 4 carbon atoms. In a further aspect, an organic residue can comprise 2 to 18 carbon atoms, 2 to 15, carbon atoms, 2 to 12 carbon atoms, 2 to 8 carbon atoms 2 to 4 carbon atoms, or 2 to 4 carbon atoms.

A very close synonym of the term “residue” is the term “radical,” which as in the specification and concluding claims, refers to a fragment, group, or substructure of a molecule described herein, regardless of how the molecule is prepared. For example, a 2,4-thiazolidinedione radical in a particular compound has the structure:

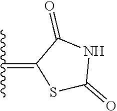 [SMILES: *=C1SC(=O)NC1=O]

regardless of whether thiazolidinedione is used to prepare the compound. In some embodiments the radical (for example an alkyl) can be further modified (i.e., substituted alkyl) by having bonded thereto one or more “substituent radicals.” The number of atoms in a given radical is not critical to the present invention unless it is indicated to the contrary elsewhere herein.

“Organic radicals,” as the term is defined and used herein, contain one or more carbon atoms. An organic radical can have, for example, 1-26 carbon atoms, 1-18 carbon atoms, 1-12 carbon atoms, 1-8 carbon atoms, 1-6 carbon atoms, or 1-4 carbon atoms. Ina further aspect, an organic radical can have 2-26 carbon atoms, 2-18 carbon atoms, 2-12 carbon atoms, 2-8 carbon atoms, 2-6 carbon atoms, or 24 carbon atoms. Organic radicals often have hydrogen bound to at least some of the carbon atoms of the organic, radical, One example, of an organic radical that comprises no inorganic atoms is a 5, 6, 7, 8-tetrahydro-2-naphthyl radical. In some embodiments, an organic radical can contain 1-10 inorganic heteroatoms bound thereto or therein, including halogens, oxygen, sulfur, nitrogen, phosphorus, and the like. Examples of organic radicals include but are not limited to an alkyl, substituted alkyl, cycloalkyl, substituted cycloakyl, mono-substituted amino di-substituted amino, acyloxy, cyano, carboxy, carboalkoxy, alkylcarboxamide, substituted alkylcarboxamide, dialkylcarboxamide, substituted diaikyicarboxamide, alkylsufonyl, alkylsulfinyl, thioalkyl, thiohaloalkyl, alkoxy, substituted alkoxy, haloalkyl, haloalkoxy, aryl, substituted aryl, heteroaryl, heterocyclic, or substituted heterocyclic radicals, wherein the terms are defined elsewhere herein. A few non-limiting examples of organic radicals that include heteroatoms include alkoxy radicals, trinuoromethoxy radicals, acetoxy radicals, dimethylamino radicals and the like.

“Inorganic radicals,” as the term is defined and used herein, contain no carbon atoms and therefore comprise only atoms other than carbon. Inorganic radicals comprise bonded combinations of atoms selected from hydrogen, nitrogen, oxygen, silicon, phosphorus, sulfur, selenium, and halogens such as fluorine, chlorine, bromine, and iodine, which can be present individually or bonded together in their chemically stable combinations, inorganic radicals have 10 or fewer, or preferably one to six or one to four inorganic atoms as listed above bonded together. Examples of inorganic radicals include, but not limited to, amino, hydroxy, halogens, nitro, thiol, sulfate, phosphate, and like commonly known inorganic radicals. The inorganic radicals do not have bonded therein the metallic elements of the periodic table (such as the alkali metals, alkaline earth metals, transition metals, lanthanide metals, or actinide metals), although such metal ions can sometimes serve as a pharmaceutically acceptable cation for anionic inorganic radicals such as a sulfate, phosphate, or like anionic inorganic radical. Inorganic radicals do not comprise metalloids elements such as boron, aluminum, gallium, germanium, arsenic, tin, lead, or tellurium, or the noble gas elements, unless otherwise specifically indicated elsewhere herein.

Compounds described herein can contain one or more double bonds and, thus, potentially give rise to cis/trans (E/Z) isomers, as well as other conformational isomers. Unless stated to the contrary, the invention includes all such possible isomers, as well as mixtures of such isomers.

Unless stated to the contrary, a formula with chemical bonds shown only as solid lines and not as wedges or dashed lines contemplates each possible isomer, e.g., each enantiomer and diastereomer, and a mixture of isomers, such as a racemic or scalemic mixture. Compounds described herein can contain one or more asymmetric centers and, thus, potentially give rise to diastereomers and optical isomers. Unless stated to the contrary, the present invention includes all such possible diastereomers as well as their racemic mixtures, their substantially pure resolved enantiomers, all possible geometric isomers, and pharmaceutically acceptable salts thereof. Mixtures of stereoisomers, as well as isolated specific stereoisomers, are also included, During the course of the synthetic procedures used to prepare such compounds, or in using racemization or epimerization procedures known to those skilled in the art, the products of such procedures can be, mixture of stereoisomers.

Many organic compounds exist in optically active forms having the ability to rotate the plane of plane-polarized light. In describing an optically active compound, the prefixes D and L or R and S are used to denote the absolute configuration of the molecule about its chiral center(s). The prefixes d and I or (+) and (−) are employed to designate the sign of rotation of plane-polarized light by the compound, with (−) or meaning that the compound is levorotatory. A compound prefixed with (+) or d is dextrorotatory. For a given chemical structure, these compounds, called stereoisomers, are identical except that they are non-superimposable mirror images of one another. A specific stereoisomer can also be referred to as, an enantiomer, and a mixture of such isomers is often called an enantiomeric mixture. A 50:50 mixture of enantiomers is referred to as a racemic mixture. Many of the compounds described herein can have one or more chiral centers and therefore can exist in different enantiomeric forms. If desired, a chiral carbon can be designated with an asterisk (*). When bonds to the chiral carbon are depicted as straight lines in the disclosed formulas, it is understood that both the (R) and (S) configurations of the chiral carbon, and hence both enantiomers and mixtures thereof, are embraced within the formula. As is used in the art, when it is desired to specify the absolute configuration about a chiral carbon, one of the bonds to the chiral carbon can be depicted as a wedge (bonds to atoms above the plane) and the other can be depicted as a series or wedge of short parallel lines is (bonds to atoms below the plane). The Cahn-Ingold-Prelog system can be used to assign the (R) or (S) configuration to a chiral carbon.

Compounds described herein comprise atoms in both their natural isotopic abundance and in non-natural abundance. The disclosed compounds can be isotopically-labeled or isotopically-substituted compounds identical to those described, but for the fact that one or more atoms are replaced by an atom having an atomic mass or mass number different from the atomic mass or mass number typically found in nature. Examples of isotopes that can be incorporated into compounds of the invention include isotopes of hydrogen, carbon, nitrogen, oxygen, sulfur, fluorine and chlorine, such as ²H, ³H, ¹³C, ¹⁴C, ¹⁵N, ¹⁸O, ¹⁷O, ³⁵S, ¹⁸F, and ³⁶Cl, respectively, Compounds further comprise prodrugs thereof and pharmaceutically acceptable salts of said compounds or of said prodrugs which contain the aforementioned isotopes and/or other isotopes of other atoms are within the scope of this invention. Certain isotopically-labeled compounds of the present invention, for example those into which radioactive isotopes such as ³H and ¹⁴C are incorporated, are useful in drug and/or substrate tissue distribution assays, Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labeled compounds of the present invention and prodrugs thereof can generally be prepared by carrying out the procedures below, by substituting a readily available isotopically labeled reagent for a non-isotopically labeled reagent.

The compounds described in the invention can be present as a solvate, in some cases, the solvent used to prepare the solvate is an aqueous solution, and the solvate, is then often referred to as a hydrate. The compounds can be present as a hydrate, which can be obtained, for example, by crystallization from a solvent or from aqueous solution. In this connection, one, two, three or any arbitrary number of solvent or water molecules can combine with the compounds according to the invention to form solvates and hydrates. Unless stated to the contrary, the invention includes all such possible solvates.

It is also appreciated that certain compounds described herein can be present as an equilibrium of tautomers. For example, ketones with an α-hydrogen can, exist in an equilibrium of the keto form and the enol form.

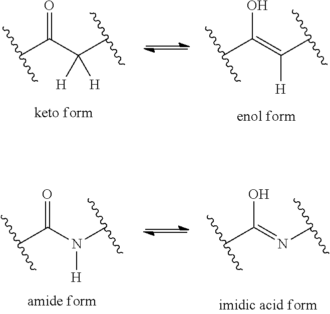 [SMILES: *CC(*)=O ; */C=C(/*)O ; *NC(*)=O ; */N=C(/*)O]

Likewise, amides with an N-hydrogen can exist in an equilibrium of the amide form and the imidic acid form. Unless stated to the contrary, the invention includes all such possible tautomers.

In some aspects, a structure of a compound can be represented by a formula:

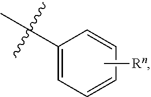

which is understood to be equivalent to a formula:

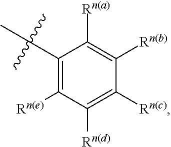

wherein n is typically an integer. That is, Rⁿ is understood to represent five independent substituents, R^{n(o)} and R^{n(b)}, R^{n(c)}, R^{n(d)}, and R^{n(e)}. By “independent substituents,” it is meant that each R substituent can be independently defined. For example, if in one instance R^{n(a)} is halogen, then R^{n(b)} is not necessarily halogen in that instance.

Certain materials, compounds, compositions, and components disclosed herein can be obtained commercially or readily synthesized using techniques generally known to those of skill in the art. For example, the starting materials and reagents used in preparing the disclosed compounds and compositions are either available from commercial suppliers such as Aldrich Chemical Co., (Milwaukee, Wis.), Acros Organics (Morris Plains, N.J.), Fisher Scientific (Pittsburgh, Pa.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991); March's Advanced Organic Chemistry, (John Wiley and Sons, 4th Edition); and Larock's Comprehensive Organic Transformations (VCH Publishers Inc., 1989).

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of embodiments described in the specification.

Disclosed are the components to be used to prepare the compositions of, the invention as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary, Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example the sub-group of A-E, B-F, and C-E would be considered disclosed. This concept applies to all aspects of this application including but not limited to, steps in methods of making and using the compositions of the invention, Thus, if there are a variety of additional steps that can be performed it is understood that each of these additional steps can be performed with any specific embodiment or combination of embodiments of the methods of the invention.

It is understood that the compositions disclosed herein have certain functions. Disclosed herein are certain structural requirements for performing the disclosed functions, and it is understood that there are a variety of structures that can perform the same function that are related to the disclosed structures, and that these structures will typically achieve the same result.

In various aspects, it is contemplated herein that the disclosed compounds further comprise their biosteric equivalents. The term “bioisosteric equivalent” refers to compounds or groups that possess near equal molecular shapes and volumes, approximately the same distribution of electrons, and which exhibit similar physical and biological properties. Examples of such equivalents are: (i) fluorine vs. hydrogen, (ii) oxo vs. Chia, (iii) hydroxyl vs. amide, (iv) carbonyl vs. oxime, (v) carboxylate vs, tetrazole. Examples of such bioisosteric replacements can be found in the literature and examples of such are: (i) Burger A, Relation of chemical structure and biological activity; in Medicinal Chemistry Third ed., Burger A, ed.; Wiley-Interscience; New York, 1970, 64-80; (ii) Burger, A.; “Isosterism and bioisosterism in drug design”; Prog. Drug Res, 1991, 37, 287-371; (iii) Burger A, “Isosterism, and bioanalogy in drug design”, Med, Chem. Res. 1994, 4, 89-92; (iv) Clark R D, Ferguson A M, Cramer R D, “Bioisosterism and molecular diversity”, Perspect. Drug Discovery Des. 1998, 9/10/11, 213-224; (v) Koyanagi T, Haqa T, “Bioisosterism in agrochemicals”, ACS Symp, Ser. 1995, 584, 15-24; (vi) Kubinyi H, “Molecular similarities, Part 1. Chemical structure and biological activity”, Pharm. Unserer Zeit 1998, 27, 92-106; (vii) Lipinski C A.; “Bioisosterism in drug design”; Annu. Rep. Med. Chem. 1986, 21, 283-91; (viii) Patani G A, LaVoie E J, “Bioisosterism: A rational approach in drug design”, Chem. Rev. (Washington, D.C.) 1996, 96, 3147-3176; (ix) Soskic V, Joksimovic J, “Bioisosteric approach in the design of new dopaminergic/serotonergic ligands”, Curr. Med. Chem. 1998, 5, 493-512 (x) Thornber C W, “Isosterism and molecular modification in drug design”, Chem. Soc. Rev. 1979, 8, 563-80.

In further aspects, bioisosteres are atoms, ions, or molecule which the peripheral layers of electrons can be considered substantially identical. The term bioisostere is usually used to mean a portion of an overall molecule, as opposed to the entire molecule itself. Bioisosteric replacement involves using one bioisostere to replace another with the expectation of maintaining or slightly modifying the biological activity of the first bioisostere. The bioisosteres in this case are thus atoms or groups of atoms having similar size, shape and electron density. Preferred bioisosteres of esters, amides or carboxylic acids are compounds containing two sites for hydrogen bond acceptance. In one embodiment, the ester, amide or carboxylic, acid bioisostere is a 5-membered monocyclic heteroaryl ring, such as an optionally substituted 1H-imidazolyl, an optionally substituted oxazolyl, 1H-tetrazolyl, [1,2,4]triazolyl, or an optionally substituted [1,2,4]oxadiazolyl.

In various aspects, the disclosed compounds cat be in the form of a co-crystal. The term “co-crystal” means a physical association of two or more molecules which owe their stability through non-covalent interaction. One or n ore components of this molecular complex provide a stable framework in the crystalline lattice. In certain instances, the guest molecules are incorporated in the crystalline lattice as anhydrates or solvates, see e.g. “Crystal Engineering of the Composition of Pharmaceutical Phases. Do Pharmaceutical Co-crystals Represent a New Path to Improved Medicines?” Almarasson, O., et. al., The Royal Society of Chemistry, 1889-18%, 2004. Preferred co-crystals include p-toluenesulfonic acid and benzenesulfonic acid.

The term “pharmaceutically acceptable co-crystal” means one that is compatible with the other ingredients of the formulation and not deleterious to the recipient thereof.

In a further aspect, the disclosed compounds can be isolated as solvates and, in particular, as hydrates of a disclosed compound, which can be obtained, for example, by crystallization from a solvent or from aqueous solution. In this connection, one, two, three or any arbitrary number of solvate or water molecules can combine with the compounds according to the disclosure to form solvates and hydrates.

The disclosed compounds can be used in the form of salts derived from inorganic or organic acids, Pharmaceutically acceptable salts include salts of acidic or basic groups present in the disclosed compounds. Suitable pharmaceutically acceptable salts include base addition salts, including alkali metal salts, e.g., sodium or potassium salts; alkaline earth metal salts, e.g., calcium or magnesium salts; and salts formed with suitable organic ligands, e.g., quaternary ammonium salts, which may be similarly prepared by reacting the drug compound with a suitable pharmaceutically acceptable base. The salts can be prepared in situ during the final isolation and purification of the compounds of the present disclosure; or following final isolation by reacting a free base function, such as a secondary or tertiary amine, of a disclosed compound with a suitable inorganic or organic acid; or reacting a free acid function, such as a carboxylic acid, of a disclosed compound with a suitable inorganic or organic base.

As used herein, “KRAS” and “K-Ras” can be used interchangeably, and refer to an enzyme encoded by a gene in humans with a cytogenetic location of 12p12.1 and a molecular location of base pairs 25,205,245 to 25,250,928 on chromosome 12 (Homo sapiens Annotation Release 109, GRCh38.p12). The gene structure in humans comprises 4 exons. KRAS has an intracellular location within the cell membrane and catalyzes GTP hydrolysis as well as playing roles in the regulation of cell proliferation and inducing transcriptional silencing of tumor suppressor genes. KRAS' has also been referred to as C-K-RAS, c-K-ras protein, G-K-ras2 protein, c-Kirsten-ras protein, cellular c-Ki-ras2 proto-oncogene, K-ras p21 protein, KI-RAS, Kirsten rat sarcoma, viral oncogene homolog, KRAS1, PR310 c-K-ras oncogene, RASK2, RASK_HUMAN, transforming protein p21 and v-Ki-ras2 Kirsten rat sarcoma viral oncogene homolog.

As used herein, “administering” can refer to an administration that is oral, topical, intravenous, subcutaneous, transcutaneous, transdermal, intramuscular, intra-joint, parenteral, intra-arteriole, intradermal, intraventricular, intraosseous, intraocular, intracranial, intraperitoneal, intralesional, intranasal, intracardiac, intraarticular, intracavernous, intrathecal, intravitreal, intracerebral, and intracerebroventricular; intratympanic, intracochlear, rectal, vaginal, by inhalation, by catheters, stents or via an implanted reservoir or other device that administers, either actively or passively (e.g. by diffusion) a composition the perivascular space and adventitia. For example a medical device such as a stent can contain a composition or formulation disposed on its surface, which can then dissolve or be otherwise distributed to the surrounding tissue and cells. The term “parenteral” can include subcutaneous, intravenous, intramuscular, intra-articular, intra-synovial, intrasternal, intrathecal, intrahepatic, intralesional, and intracranial injections or infusion techniques. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

As used herein, “therapeutic agent” can refer to any substance, compound, molecule, and the like, which can be biologically active or otherwise can induce a pharmacologic, immunogenic, biologic and for physiologic effect on a subject to which it is administered to by local and/or systemic action. A therapeutic agent can be a primary active agent, or in other words, the component(s) of a composition to which the whole or part of the effect of the composition is attributed. A therapeutic agent can be a secondary therapeutic agent, or in other words, the component(s) of a composition to which an additional part and/or other effect of the composition is attributed. The term therefore encompasses those compounds or chemicals traditionally regarded as drugs, vaccines, and biopharmaceuticals including molecules such as proteins, peptides, hormones, nucleic acids, gene constructs and the like. Examples of therapeutic agents are described in well-known literature references such as the Merck Index (14th edition), the Physicians' Desk Reference (64th edition), and The Pharmacological Basis of Therapeutics (12th edition), and they include, without limitation, medicaments: vitamins; mineral supplements; substances used for the treatment, prevention, diagnosis, cure or mitigation of a disease or illness; substances that affect the structure or function of the body, or pro-drugs, which become biologically active or more active after they have been placed in a physiological environment. For example, the term “therapeutic agent” includes compounds or compositions for use in all of the major therapeutic areas including, but not limited to, adjuvants; anti-infectives such as antibiotics and antiviral agents; analgesics and analgesic combinations, anorexics, anti-inflammatory agents, anti-epileptics, local and general anesthetics, hypnotics, sedatives, antipsychotic agents, neuroleptic agents, antidepressants, anxiolytics, antagonists, neuron blocking agents, anticholinergic and cholinomimetic agents, antimuscarinic and muscarinic agents, antiadrenergic, antiarrhythmics, antihypertensive agents, hormones, and nutrients, antiarthritic, antiasthmatic agents: anticonvulsants, antihistamines, antinauseants, antineoplastics, antipruritics, antipyretics; antispasmodics, cardiovascular preparations (including calcium channel blockers, beta-blockers, beta-agonists and antiarrhythmics), antihypertensives, diuretics, vasodilators; central nervous system stimulants: cough and cold preparations; decongestants; diagnostics; hormones; bone growth stimulants and bone resorption inhibitors; immunosuppressives; muscle relaxants; psychostimulants: sedatives; tranquilizers; proteins, peptides, and fragments thereof (whether naturally occurring, chemically synthesized or recombinantly produced); and nucleic acid molecules (polymeric forms of two or more nucleotides, either ribonucleotides (RNA) or deoxyribonucleotides (DNA) including both double- and single-stranded molecules, gene constructs, expression vectors, antisense molecules and the like), small molecules (e.g., doxorubicin) and other biologically active macromolecules such as, for example, proteins and enzymes. The agent may be a biologically active agent used in medical, including veterinary, applications and in agriculture, such as with plants, as well as other areas. The term therapeutic agent also includes without limitation, medicaments; vitamins; mineral supplements: substances used for the treatment, prevention, diagnosis, cure or mitigation of disease or illness; or substances which affect the structure or function of the body; or pro-drugs, which become biologically active or more active after they have been placed in a predetermined physiological environment.

As used herein, “kit” means a collection of at least two components constituting the kit. Together, the components constitute a functional unit for a given purpose. Individual member components may be physically packaged together or separately. For example, a kit comprising an instruction for using the kit may or may not physically include the instruction with other individual member components, Instead, the instruction can be supplied as a separate member component, either in a paper form or an electronic form which may be supplied on computer readable memory device or downloaded from an internet website, or as recorded presentation.

As used herein, “instruction(s)” means documents describing relevant materials or methodologies pertaining to a kit. These materials may include any combination of the following: background information, list of components and their availability information (purchase information, etc.), brief or detailed protocols for using the kit, trouble-shooting, references, technical support, and any other related documents. Instructions can be supplied with the kit or as a separate member component, either as a paper form or an electronic form which may be supplied on computer readable memory device or downloaded from an Internet website, or as recorded presentation. Instructions can comprise one or multiple documents, and are meant to include future updates.

As used herein, “attached” can refer to covalent or non-covalent interaction between two or more molecules, Non-covalent interactions can include ionic bonds, electrostatic interactions, van der Walls forces, dipole-dipole interactions, dipole-induced-dipole interactions, London dispersion forces, hydrogen bonding, halogen bonding, electromagnetic interactions, TT-TT interactions, cation-TT interactions, anion-TT interactions, polar TT-Interactions, and hydrophobic effects.

As used interchangeably herein, “subject,” “individual,” or “patient” can refer to a vertebrate organism, such as a mammal (e.g, human), “Subject” can also refer to a cell, a population of cells, a tissue, an organ, or an organism, preferably to human and constituents thereof.

As used herein the terms “reefing” and “treatment” can refer generally to obtaining a desired pharmacological and/or physiological effect. The effect can be, but does not necessarily have to be, prophylactic in terms of preventing or partially preventing a disease, symptom or condition thereof, such as pancreatic ductal adenocarcinoma, gastric cancer, and/or other cancers and/or tumors. The effect can be therapeutic in terms of a partial or complete cure of a disease, condition, symptom or adverse effect attributed to the disease, disorder, or condition. The term “treatment” as used herein can include any treatment of pancreatic ductal adenocarcinoma, gastric cancer, and/or other cancers and/or tumors in a subject, particularly a human and can include any one or more of the following: (a) preventing the disease from occurring in a subject which may be predisposed to the disease but has not yet been diagnosed as having it; (b) inhibiting the disease, i.e., arresting its development; and (c) relieving the disease, i.e., mitigating or ameliorating the disease and/or its symptoms or conditions. The term “treatment” as used herein can refer to both therapeutic treatment alone, prophylactic treatment alone, or both therapeutic and prophylactic treatment Those in need of treatment (subjects in need thereof) can include those already with the disorder and/or those in which the disorder is to be prevented. As used herein, the term “treating”, can include inhibiting the disease, disorder or condition, e.g., impeding its progress; and relieving the disease, disorder, or condition, e.g., causing regression of the disease, disorder and/or condition. Treating the disease, disorder, or condition can include ameliorating at least one symptom of the particular disease, disorder, or condition, even if the underlying pathophysiology is not affected, e.g., such as treating the pain of a subject by administration of an analgesic agent even though such agent does not treat the cause of the pain.

As used herein, “dose,” “unit dose,” or “dosage” can refer to physically discrete units suitable for use in a subject, each unit containing a predetermined quantity of a disclosed compound and/or a pharmaceutical composition thereof calculated to produce the desired response or responses in association with its administration.

As used herein, “therapeutic” can refer to treating, healing, and/or ameliorating a disease, disorder, condition, or side effect, or to decreasing in the rate of advancement of a disease, disorder, condition, or side effect.

As used herein, “effective amount” can refer to the amount of a disclosed compound or pharmaceutical composition provided herein that is sufficient to effect beneficial or desired biological, emotional, medical, or clinical response of a cell, tissue, system, animal, or human. An effective amount can be administered in one or more administrations, applications, or dosages. The term can also include within its scope amounts effective to enhance or restore to substantially normal physiological function.

As used herein, the term “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder: the specific composition employed; the age, body weight, general health, sex and diet of the patient; the time of administration; the route of administration; the rate of excretion of the specific compound employed: the duration of the treatment; drugs used in combination or coincidental with the specific compound employed and like factors within the knowledge and expertise of the health practitioner and which may be well known in the medical arts. In the case of treating a particular disease or condition, in some instances, the desired response can be inhibiting the progression of the disease or condition. This may involve only slowing the progression of the disease temporarily. However, in other instances, it may be desirable to halt the progression of the disease permanently. This can be monitored by routine diagnostic methods known to one of ordinary skill in the art for any particular disease. The desired response to treatment of the disease or condition also can be delaying the onset or even preventing the onset of the disease or condition.

For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. It is generally preferred that a maximum dose of the pharmacological agents of the invention (alone or in combination with other therapeutic agents) be used, that is, the highest safe dose according to sound medical judgment. It will be understood by those of ordinary skill in the art however, that a patient may insist upon a lower dose or tolerable dose for medical reasons, psychological reasons or for virtually any other reasons.

A response to a therapeutically effective dose of a disclosed compound and/or pharmaceutical composition, for example, can be measured by determining the physiological effects of the treatment or medication, such as the decrease or lack of disease symptoms following administration of the treatment or pharmacological agent. Other assays will be known to one of ordinary skill in the art and can be employed for measuring the level of the response. The amount of a treatment may be varied for example by increasing or decreasing the amount of a disclosed compound and/or pharmaceutical composition, by changing the disclosed compound and/or pharmaceutical composition administered, by changing the route of administration, by changing the dosage timing and so on. Dosage can vary, and can be administered in one or more dose administrations daily, for one or several days. Guidance can be found in the literature for appropriate dosages for given classes of pharmaceutical products.

As used herein, the term “prophylactically effective amount” refer n amount effective for preventing onset or initiation of a disease or condition.

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

The term “pharmaceutically acceptable” describes a material that is not biologically or otherwise undesirable, i.e., without causing an unacceptable level of undesirable biological, effects or interacting in a deleterious manner.

The term “pharmaceutically acceptable salts”, as used herein, means salts of the active principal agents which are prepared with acids or bases that are tolerated by a biological system or tolerated by a subject or tolerated by a biological system and tolerated by a subject when administered in a therapeutically effective amount. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include, but are not limited to; sodium, potassium, calcium, ammonium, organic amino, magnesium salt, lithium salt, strontium salt or a similar salt. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include, but are not limited to, those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like.

The term “pharmaceutically acceptable ester” refers to esters of compounds of the present disclosure which hydrolyze in vivo and include those that break down readily in the human body to leave the parent compound or a salt thereof. Examples of pharmaceutically acceptable, non-toxic esters of the present disclosure include C1-to-C6 alkyl esters and C 5-to-C7 cycloalkyl esters, although C1-to-C4 alkyl esters are preferred. Esters of disclosed compounds can be prepared according to conventional methods. Pharmaceutically acceptable esters can be appended onto hydroxy groups by reaction of the compound that contains the hydroxy group with acid and an alkylcarboxylic acid such as acetic acid, or with acid and an arylcarboxylic acid such as benzoic acid. In the case of compounds containing carboxylic acid groups, the pharmaceutically acceptable esters are prepared from compounds containing the carboxylic acid groups by reaction of the compound with base such as triethylamine and an alkyl halide, for example with methyl iodide, benzyl iodide, cyclopentyl iodide or alkyl triflate. They also can be prepared by reaction of the compound with an acid such as hydrochloric acid and an alcohol such as ethanol or methanol.

The term “pharmaceutically acceptable amide” refers to nontoxic amides of the present disclosure derived from ammonia, primary C1-to-C6 alkyl amines and secondary C 1-to-C6 dialkyl amines. In the case of secondary amines, the amine can also be in the form of a 5 or 6-membered heterocycle containing one nitrogen atom. Amides derived from ammonia, C1-to-C3 alkyl primary amides and C1-to-C2 dialkyl secondary amides are preferred. Amides of disclosed compounds can be prepared according to conventional methods. Pharmaceutically acceptable amides can be prepared from compounds containing primary or secondary amine groups by reaction of the compound that contains the amino group with an alkyl anhydride, aryl anhydride, acyl halide, or aroyl halide. In the case of compounds containing carboxylic acid groups, the pharmaceutically acceptable amides are prepared from compounds containing the carboxylic acid groups by reaction of the compound with base such as triethylamine, a dehydrating agent such as dicyclohexyl carbodiimide, or carbonyl diimidazole, and an alkyl amine, dialkylamine, for example with methylamine, diethylamine, and piperidine. They also can be prepared by reaction of the compound with an acid such as sulfuric acid and an alkylcarboxylic acid such as acetic acid, or with acid and an arylcarboxylic acid such as benzoic acid under dehydrating conditions such as with molecular sieves added. The composition can contain a compound of the present disclosure in the form of a pharmaceutically acceptable prodrug.

The term “pharmaceutically acceptable prodrug” or “prodrug” represents those prodrugs of the compounds of the present disclosure which are, within the scope of sound medical judgment, suitable for use in contact with the tissues of humans and lower anima without undue toxicity, irritation, allergic response, and the like, commensurate with a reasonable benefit/risk ratio, and effective for their intended use. Prodrugs of the present disclosure can be rapidly transformed in vivo to a parent compound having a structure of a disclosed compound, for example, by hydrolysis in blood. A thorough discussion is provided in T. Higuchi and V. StelIa, Pro-drugs as Novel Delivery Systems, V. 14 of the A.C.S. Symposium Series, and in Edward B. Roche, ed., Bioreversible Carriers in Drug Design, American Pharmaceutical Association and Pergamon Press (1987).

As used herein, the term “derivative” refers to a compound having a structure derived from the structure of a parent compound (e.g., a compound disclosed herein) and whose structure is sufficiently similar to those disclosed herein and based upon that similarity, would be expected by one skilled in the art to exhibit the same or similar activities and utilities as the claimed compounds, or to induce, as a precursor, the same or similar activities and utilities as the claimed compounds. Exemplary derivatives include salts, esters, amides, salts of esters or amides, and N-oxides of a parent compound.

The term “contacting” as used herein refers to bringing a disclosed compound or pharmaceutical composition in proximity to a cell, a target protein, or other biological entity together in such a manner that the disclosed compound or pharmaceutical composition can affect the activity of the a cell, target protein, or other biological entity, either directly; i.e., by interacting with the cell, target protein, or other biological entity itself, or indirectly; i.e., by interacting with another molecule, co-factor, factor, or protein on which the activity of the cell, target protein, or other biological entity itself is dependent.

As used herein, nomenclature for compounds, including organic compounds, can be given using common names, IUPAC, IUBMB, or CAS recommendations for nomenclature. When one or more stereochemical features are present, Cahn-Ingold-Prelog rules for stereochemistry can be employed to designate stereochemical priority, E/Z specification, and the like. One of skill in the art can readily ascertain the structure of a compound if given a name, either by systemic reduction of the compound structure using naming conventions, or by commercially available software, such as CHEMDRAW™ (Carnbridgesoft Corporation, U.S.A.).

It is understood, that unless otherwise specified, temperatures referred to herein are based on atmospheric pressure (i.e. one atmosphere).

Described herein are pyrazolopyrimidine derivatives that have therapeutic or clinical utility. Also described herein are methods of synthesizing the pyrazolopyrimidine derivatives. Also described herein are methods of administering the pyrazolopyrimidine derivatives to a subject in need thereof. In some aspects, the subject can have a cancer such as, for example, pancreatic ductal adenocarcinoma. Other compositions, compounds, methods, features, and advantages of the present disclosure will be or become apparent to one having ordinary skill in the art upon examination of the following drawings, detailed description, and examples. It is intended that all such additional compositions, compounds, methods, features, and advantages be included within this description, and be within the scope of the present disclosure.

Compounds

In one aspect, disclosed herein is a compound having a structure represented by a formula:

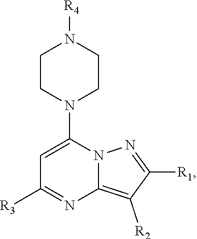

wherein R is aryl, substituted aryl, —(C1-C7 alkanediyl)-R₅, or —(C1-C7 alkanediyl)-(C═O)—R₅; wherein each of R₂ and R₃ is independently C1-C8 alkyl, cycloalkyl, heterocycloalkyl, aryl, heteroaryl, substituted aryl, or substituted heteroaryl; wherein R₄ is —(C1-C7 alkyl) or —(C—7 alkanediyl)-R₆; and wherein each of R₆ and R₅ is independently hydrogen, methoxy—SF₅, —OH, —SH, —NH₂, or halogen; or a pharmaceutically acceptable salt, solvate, or ester thereof.

In a further aspect, disclosed herein is a compound having a structure represented by a formula:

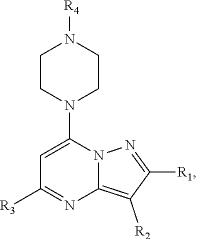

wherein R₁ is aryl, substituted aryl, —(C1-C7 alkanediyl)-R₅, or —(C1-C7 alkanediyl)-(C═O)—R₅; wherein each of R₂ and R₂ is independently C1-C8 alkyl, cycloalkyl, heterocycloalkyl, aryl, heteroaryl, substituted aryl, or substituted heteroaryl; wherein R₄ is —(C1-C7 alkyl) or —(C1-C7 alkanediyl)-R₅; and wherein each of R₅ and R₆ is independently hydrogen, methoxy, —S₅, —OH, —SH, —NH₂, or halogen; or a pharmaceutically acceptable salt, solvate, or ester thereof; provided that the compound does not have structure represented by a formula of:

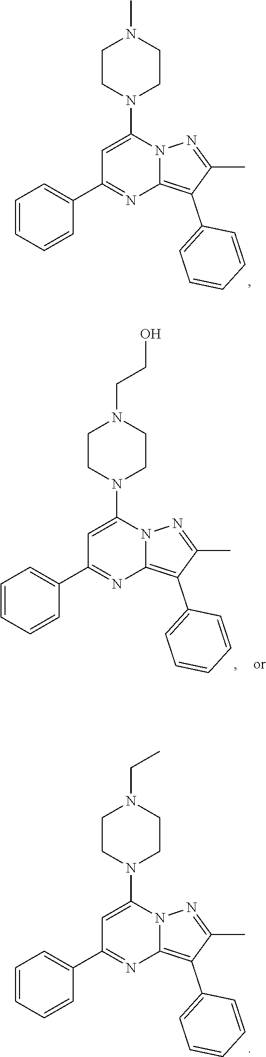 [SMILES: Cc1nn2c(N3CCN(C)CC3)cc(-c3ccccc3)nc2c1-c1ccccc1 ; Cc1nn2c(N3CCN(CCO)CC3)cc(-c3ccccc3)nc2c1-c1ccccc1 ; CCN1CCN(c2cc(-c3ccccc3)nc3c(-c4ccccc4)c(C)nn23)CC1]

In some aspects, R₁ is methyl or phenyl. In other aspects, R₂ is phenyl or 4-methoxyphenyl. In still other aspects, R₃ is phenyl, methyl, 4-methoxyphenyl, methylimidazole, or 4-fluorophenyl. In still other aspects, R₄ is —(CH₂)₂—OH, —(CH₂)₂—OCH₃, or 2-methoxyphenyl.

In another aspect, disclosed herein is a compound having a structure represented by a formula:

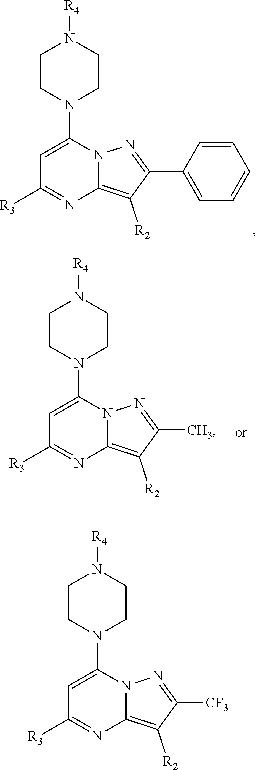

wherein R₂, R₃, and R₄ are defined as described above.

In still another aspect, disclosed herein is a compound having a structure represented by a formula:

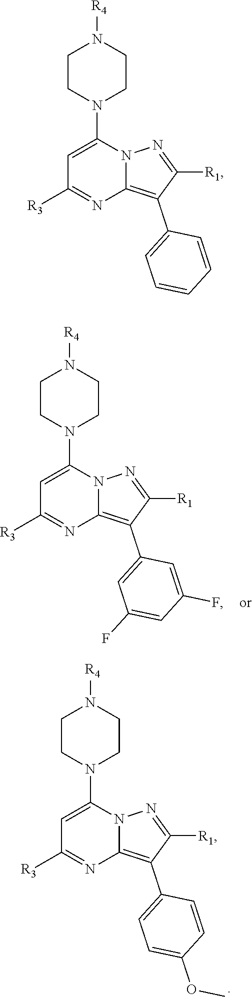

wherein R₁, R₃, and R₄ are defined as described above.

In still another aspect, disclosed herein is a co pound, having a structure represented by a formula:

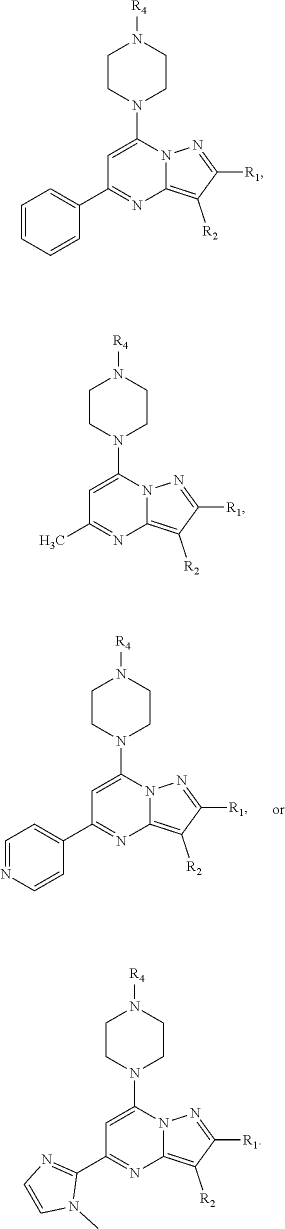

wherein R₁, R₂, and R₄ are defined as described above.

In another aspect, disclosed herein is a compound having a structure represented by a formula:

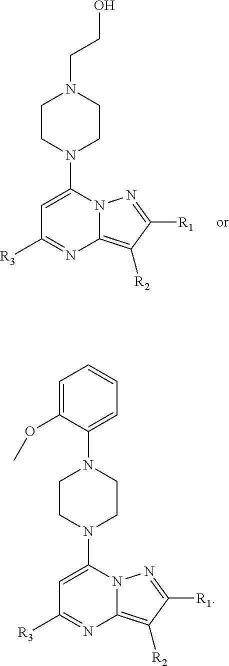

wherein R₁, R₂, and R₃ are defined as described above.

In still another aspect, disclosed herein is a compound having a structure represented, by a formula:

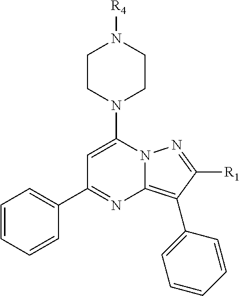

wherein R₁ is methyl, —CF₃, or phenyl and R₄ is —(CH₂)₂—OH, —(CH₂)₂—OCH₃, or 2-methoxyphenyl.

In a further aspect, disclosed herein is a compound having a structure represented by a formula;

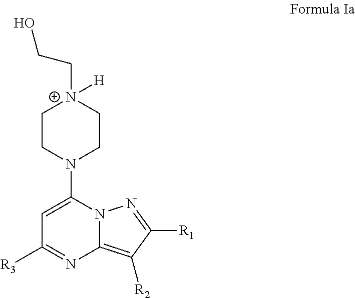

wherein R₁ is aryl, substituted aryl, —(C1-C7 alkanediyl)-R₅, or —(C1-C7 alkanediyl)-(C═O)—R₅; wherein each of R₂ and R₃ is independently C1-C8 alkyl, cycloalkyl, heterocycloalkyl, aryl, heteroaryl, substituted aryl, or substituted heteroaryl; wherein R₄ is -(C1-C7 alkyl) or —(C1-C7 alkanediyl)-R₆; and wherein each of R₅ and R₆ is independently hydrogen, methoxy, —SF₅, —OH, —SH, —NH₂, or halogen; or a pharmaceutically acceptable salt, solvate, or ester thereof.

In some embodiments, in Formula Ia, R₃ is alkyl (e.g. methyl), —CF₃, phenyl, hydroxyphenyl (preferably 4-hydroxyphenyl), or halophenyl (preferably 4-halophenyl).

In some embodiments, in Formula Ia, R₃ is a substituted phenyl.

In some embodiments, in Formula Ia R₂ is phenyl or halophenyl preferably 4-halophenyl).

In some embodiments, in Formula Ia, R₂ is a substituted phenyl.

In some embodiments in Formula Ia, R₁ is alkyl (e.g., methyl) or phenyl.

In some embodiments, in Formula Ia, R₁ is a substituted phenyl.

In further embodiments, Formula Ia is one of:

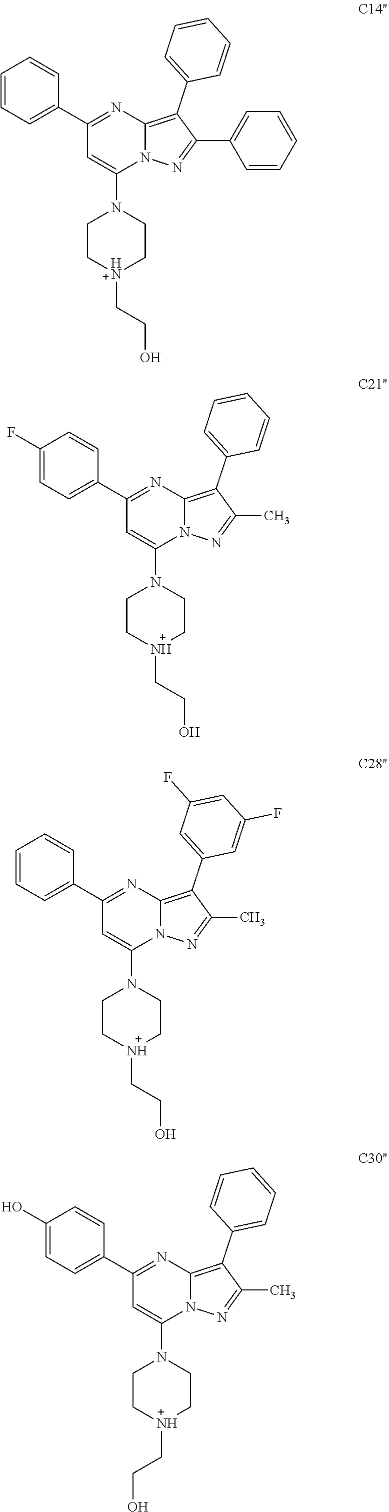

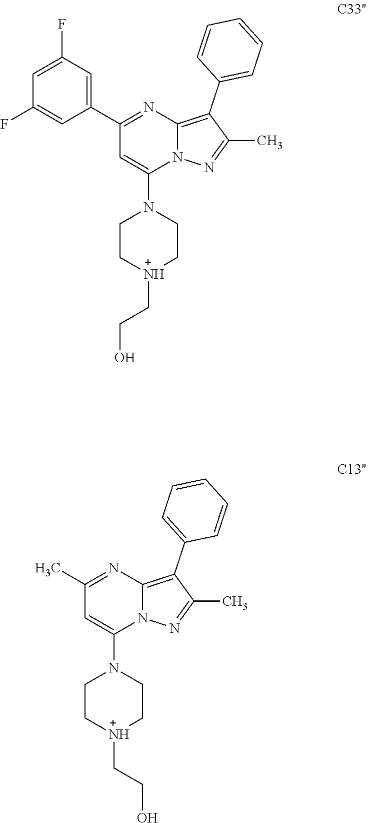 [SMILES: Cc1nn2c(N3CC[NH+](CCO)CC3)cc(-c3cc(F)cc(F)c3)nc2c1-c1ccccc1 ; Cc1cc(N2CC[NH+](CCO)CC2)n2nc(C)c(-c3ccccc3)c2n1]

In some aspects, exemplary, but non-limiting, compounds according to the present disclosure are presented below along with compound identifiers used throughout the detailed description that follows:

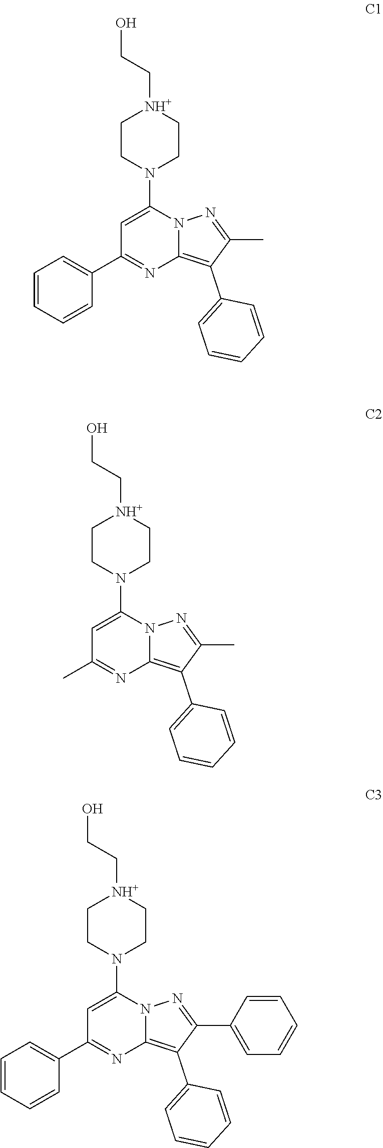 [SMILES: Cc1nn2c(N3CC[NH+](CCO)CC3)cc(-c3ccccc3)nc2c1-c1ccccc1 ; Cc1cc(N2CC[NH+](CCO)CC2)n2nc(C)c(-c3ccccc3)c2n1 ; OCC[NH+]1CCN(c2cc(-c3ccccc3)nc3c(-c4ccccc4)c(-c4ccccc4)nn23)CC1]

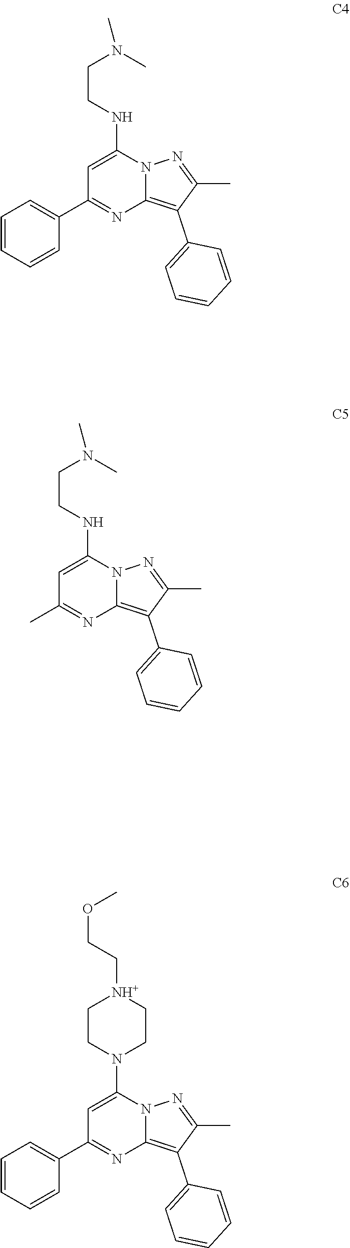 [SMILES: Cc1nn2c(NCCN(C)C)cc(-c3ccccc3)nc2c1-c1ccccc1 ; Cc1cc(NCCN(C)C)n2nc(C)c(-c3ccccc3)c2n1 ; COCC[NH+]1CCN(c2cc(-c3ccccc3)nc3c(-c4ccccc4)c(C)nn23)CC1]

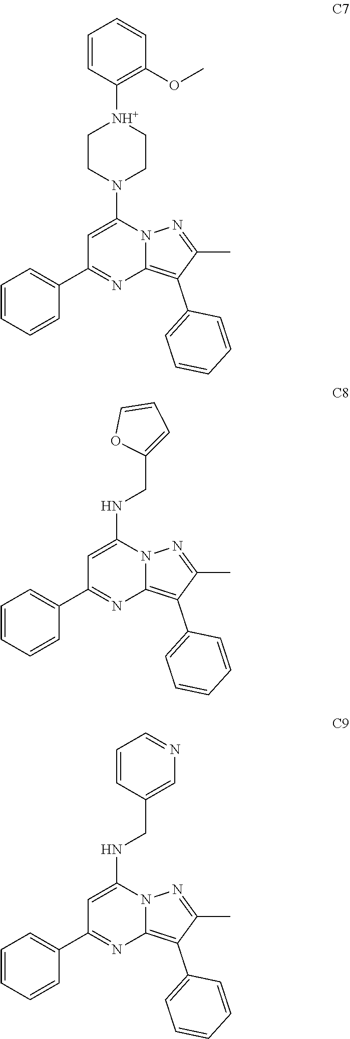 [SMILES: COc1ccccc1[NH+]1CCN(c2cc(-c3ccccc3)nc3c(-c4ccccc4)c(C)nn23)CC1 ; Cc1nn2c(NCc3ccco3)cc(-c3ccccc3)nc2c1-c1ccccc1 ; Cc1nn2c(NCc3cccnc3)cc(-c3ccccc3)nc2c1-c1ccccc1]

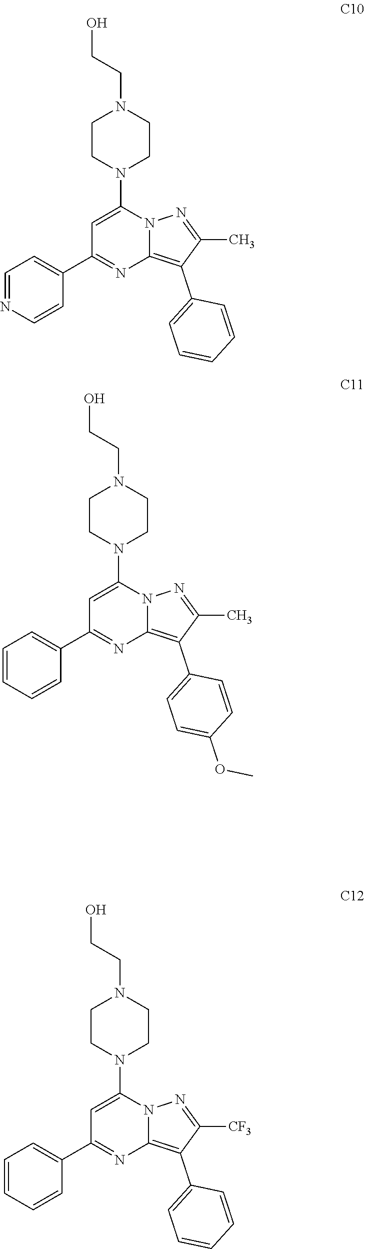 [SMILES: Cc1nn2c(N3CCN(CCO)CC3)cc(-c3ccncc3)nc2c1-c1ccccc1 ; COc1ccc(-c2c(C)nn3c(N4CCN(CCO)CC4)cc(-c4ccccc4)nc23)cc1 ; OCCN1CCN(c2cc(-c3ccccc3)nc3c(-c4ccccc4)c(C(F)(F)F)nn23)CC1]

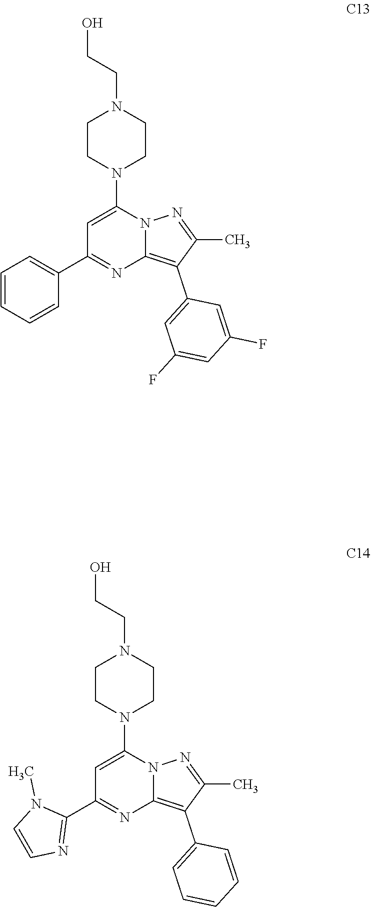 [SMILES: Cc1nn2c(N3CCN(CCO)CC3)cc(-c3ccccc3)nc2c1-c1cc(F)cc(F)c1 ; Cc1nn2c(N3CCN(CCO)CC3)cc(-c3nccn3C)nc2c1-c1ccccc1]

or a subgroup thereof.

In further aspects, exemplary, but non-limiting, compounds according to the present disclosure are presented below along with compound identifiers used throughout the detailed description that follows:

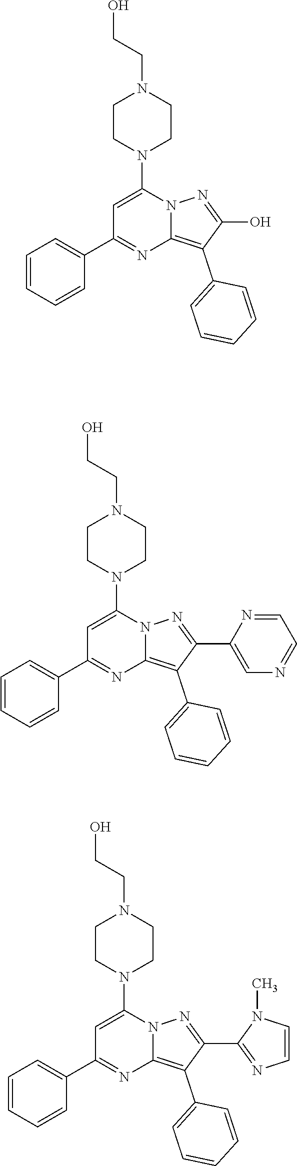 [SMILES: OCCN1CCN(c2cc(-c3ccccc3)nc3c(-c4ccccc4)c(O)nn23)CC1 ; OCCN1CCN(c2cc(-c3ccccc3)nc3c(-c4ccccc4)c(-c4cnccn4)nn23)CC1 ; Cn1ccnc1-c1nn2c(N3CCN(CCO)CC3)cc(-c3ccccc3)nc2c1-c1ccccc1]

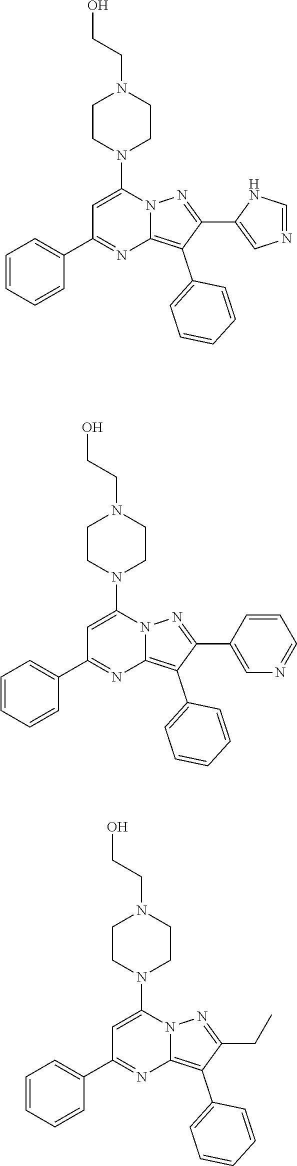 [SMILES: OCCN1CCN(c2cc(-c3ccccc3)nc3c(-c4ccccc4)c(-c4cnc[nH]4)nn23)CC1 ; OCCN1CCN(c2cc(-c3ccccc3)nc3c(-c4ccccc4)c(-c4cccnc4)nn23)CC1 ; CCc1nn2c(N3CCN(CCO)CC3)cc(-c3ccccc3)nc2c1-c1ccccc1]

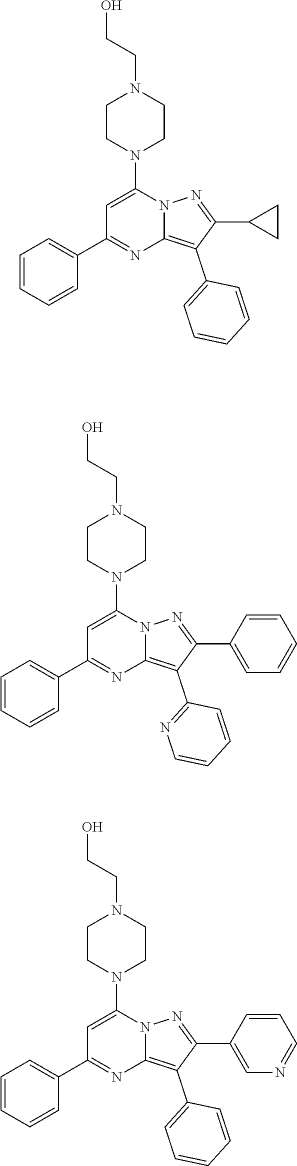 [SMILES: OCCN1CCN(c2cc(-c3ccccc3)nc3c(-c4ccccc4)c(C4CC4)nn23)CC1 ; OCCN1CCN(c2cc(-c3ccccc3)nc3c(-c4ccccn4)c(-c4ccccc4)nn23)CC1 ; OCCN1CCN(c2cc(-c3ccccc3)nc3c(-c4ccccc4)c(-c4cccnc4)nn23)CC1]

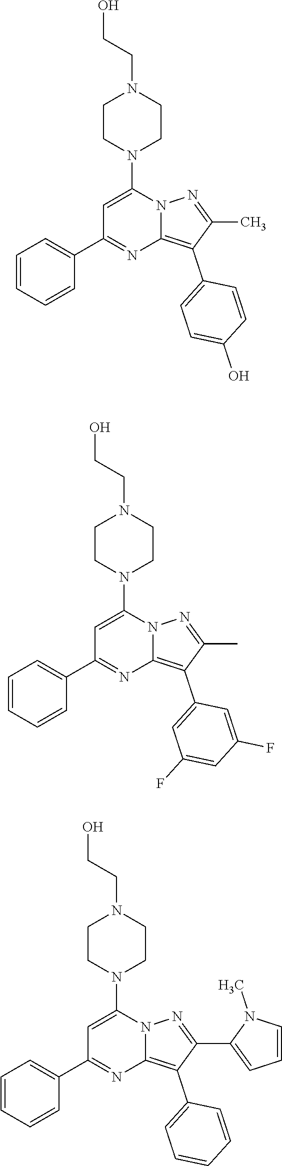 [SMILES: Cc1nn2c(N3CCN(CCO)CC3)cc(-c3ccccc3)nc2c1-c1ccc(O)cc1 ; Cc1nn2c(N3CCN(CCO)CC3)cc(-c3ccccc3)nc2c1-c1cc(F)cc(F)c1 ; Cn1cccc1-c1nn2c(N3CCN(CCO)CC3)cc(-c3ccccc3)nc2c1-c1ccccc1]

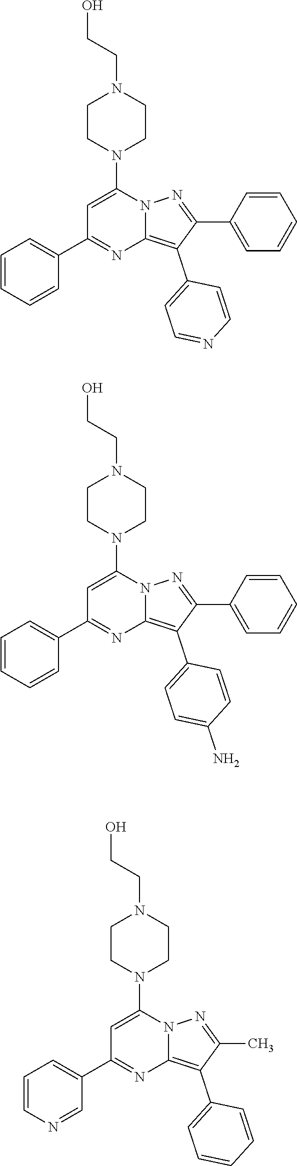 [SMILES: OCCN1CCN(c2cc(-c3ccccc3)nc3c(-c4ccncc4)c(-c4ccccc4)nn23)CC1 ; Nc1ccc(-c2c(-c3ccccc3)nn3c(N4CCN(CCO)CC4)cc(-c4ccccc4)nc23)cc1 ; Cc1nn2c(N3CCN(CCO)CC3)cc(-c3cccnc3)nc2c1-c1ccccc1]

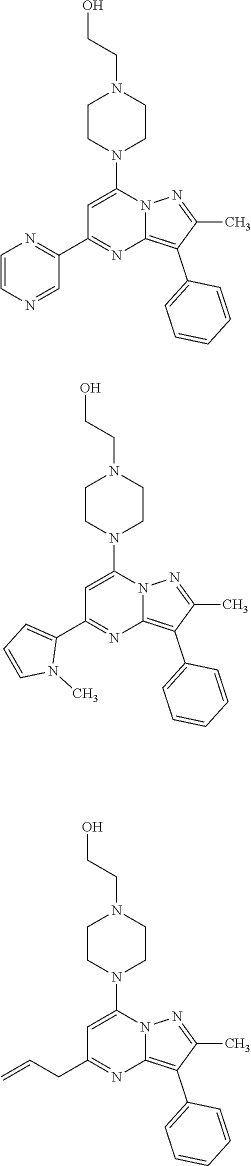 [SMILES: Cc1nn2c(N3CCN(CCO)CC3)cc(-c3cnccn3)nc2c1-c1ccccc1 ; Cc1nn2c(N3CCN(CCO)CC3)cc(-c3cccn3C)nc2c1-c1ccccc1 ; C=CCc1cc(N2CCN(CCO)CC2)n2nc(C)c(-c3ccccc3)c2n1]

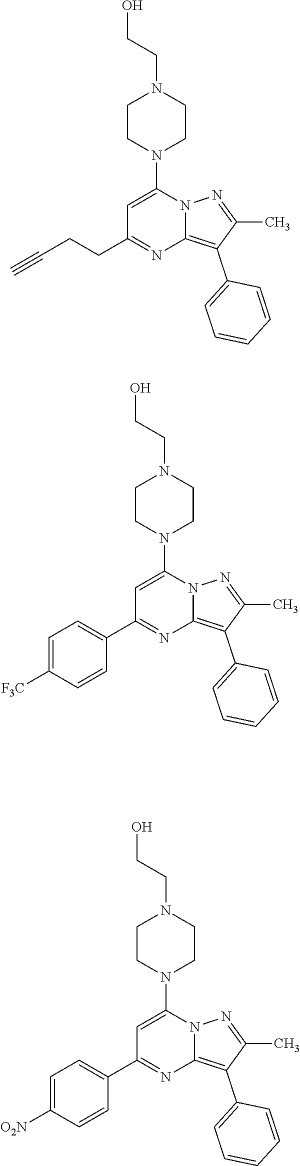 [SMILES: C#CCCc1cc(N2CCN(CCO)CC2)n2nc(C)c(-c3ccccc3)c2n1 ; Cc1nn2c(N3CCN(CCO)CC3)cc(-c3ccc(C(F)(F)F)cc3)nc2c1-c1ccccc1 ; Cc1nn2c(N3CCN(CCO)CC3)cc(-c3ccc([N+](=O)[O-])cc3)nc2c1-c1ccccc1]

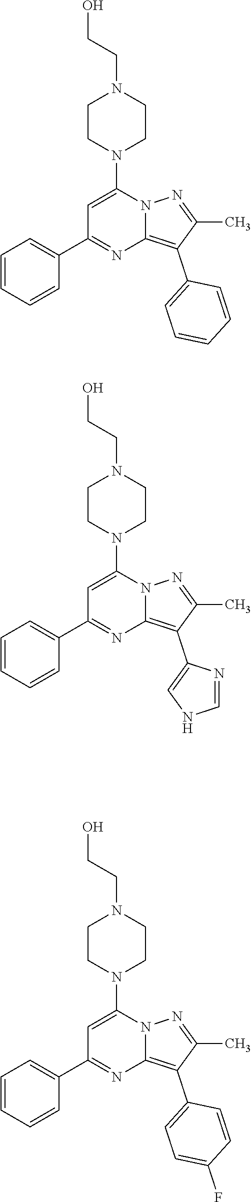 [SMILES: Cc1nn2c(N3CCN(CCO)CC3)cc(-c3ccccc3)nc2c1-c1ccccc1 ; Cc1nn2c(N3CCN(CCO)CC3)cc(-c3ccccc3)nc2c1-c1c[nH]cn1 ; Cc1nn2c(N3CCN(CCO)CC3)cc(-c3ccccc3)nc2c1-c1ccc(F)cc1]

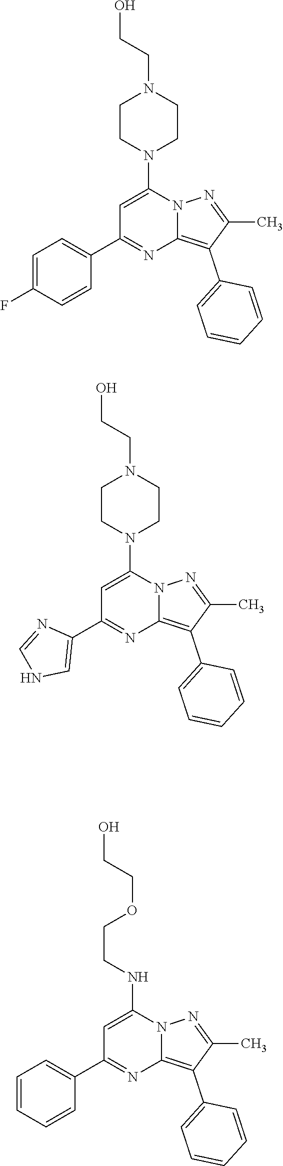 [SMILES: Cc1nn2c(N3CCN(CCO)CC3)cc(-c3ccc(F)cc3)nc2c1-c1ccccc1 ; Cc1nn2c(N3CCN(CCO)CC3)cc(-c3c[nH]cn3)nc2c1-c1ccccc1 ; Cc1nn2c(NCCOCCO)cc(-c3ccccc3)nc2c1-c1ccccc1]

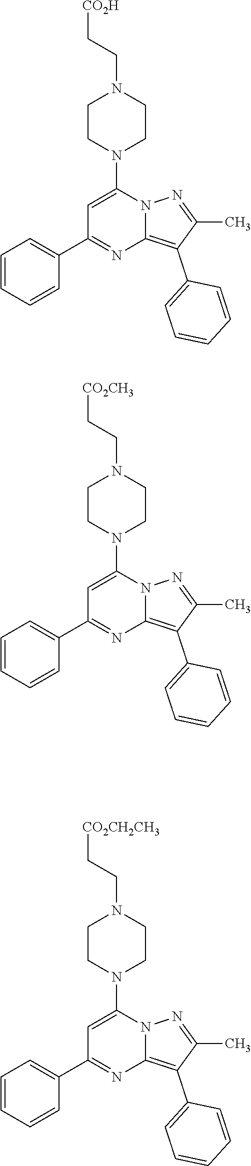

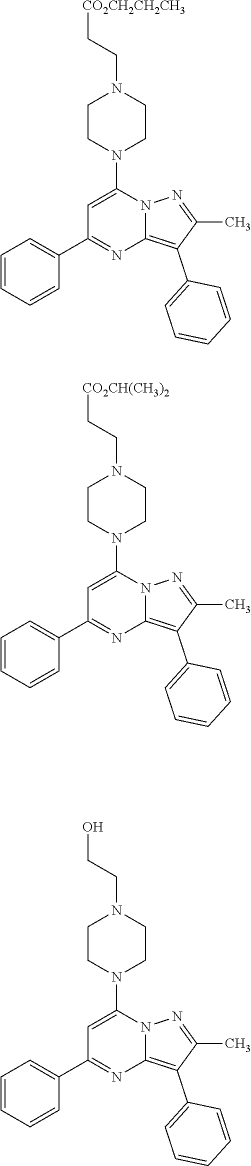

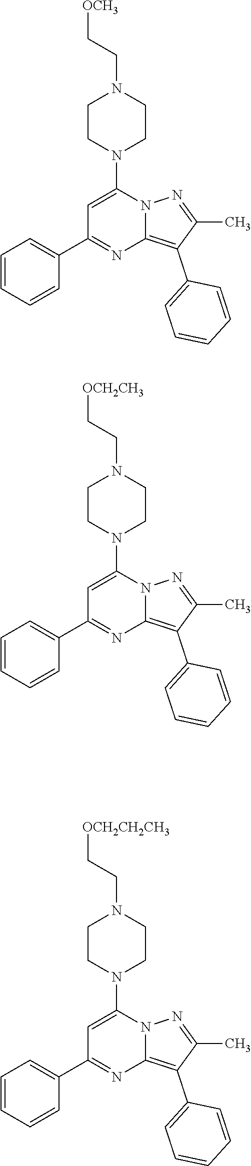

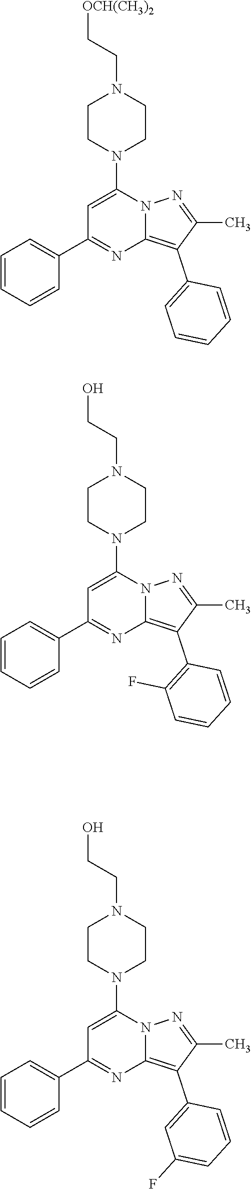

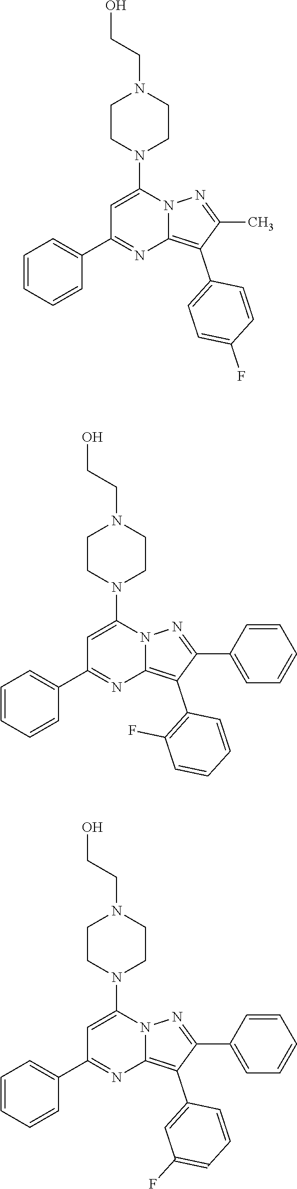 [SMILES: Cc1nn2c(N3CCN(CCO)CC3)cc(-c3ccccc3)nc2c1-c1ccc(F)cc1 ; OCCN1CCN(c2cc(-c3ccccc3)nc3c(-c4ccccc4F)c(-c4ccccc4)nn23)CC1 ; OCCN1CCN(c2cc(-c3ccccc3)nc3c(-c4cccc(F)c4)c(-c4ccccc4)nn23)CC1]

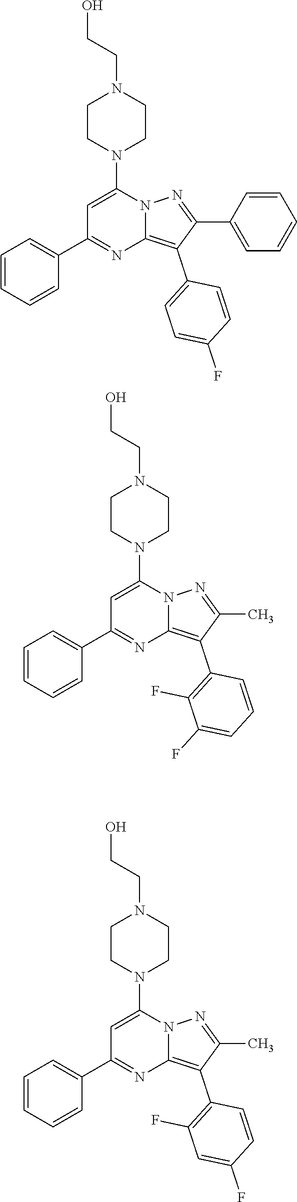 [SMILES: OCCN1CCN(c2cc(-c3ccccc3)nc3c(-c4ccc(F)cc4)c(-c4ccccc4)nn23)CC1 ; Cc1nn2c(N3CCN(CCO)CC3)cc(-c3ccccc3)nc2c1-c1cccc(F)c1F ; Cc1nn2c(N3CCN(CCO)CC3)cc(-c3ccccc3)nc2c1-c1ccc(F)cc1F]

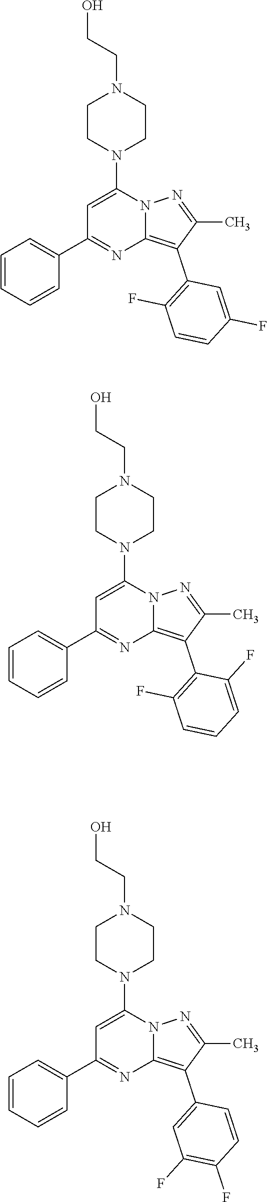 [SMILES: Cc1nn2c(N3CCN(CCO)CC3)cc(-c3ccccc3)nc2c1-c1cc(F)ccc1F ; Cc1nn2c(N3CCN(CCO)CC3)cc(-c3ccccc3)nc2c1-c1c(F)cccc1F ; Cc1nn2c(N3CCN(CCO)CC3)cc(-c3ccccc3)nc2c1-c1ccc(F)c(F)c1]

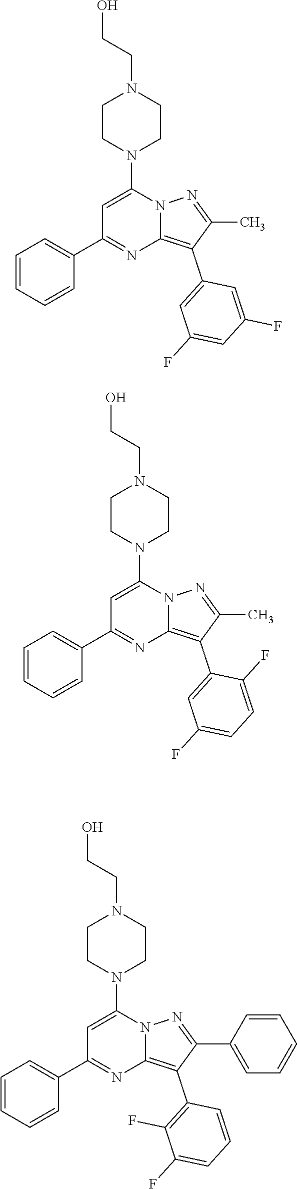 [SMILES: Cc1nn2c(N3CCN(CCO)CC3)cc(-c3ccccc3)nc2c1-c1cc(F)cc(F)c1 ; Cc1nn2c(N3CCN(CCO)CC3)cc(-c3ccccc3)nc2c1-c1cc(F)ccc1F ; OCCN1CCN(c2cc(-c3ccccc3)nc3c(-c4cccc(F)c4F)c(-c4ccccc4)nn23)CC1]

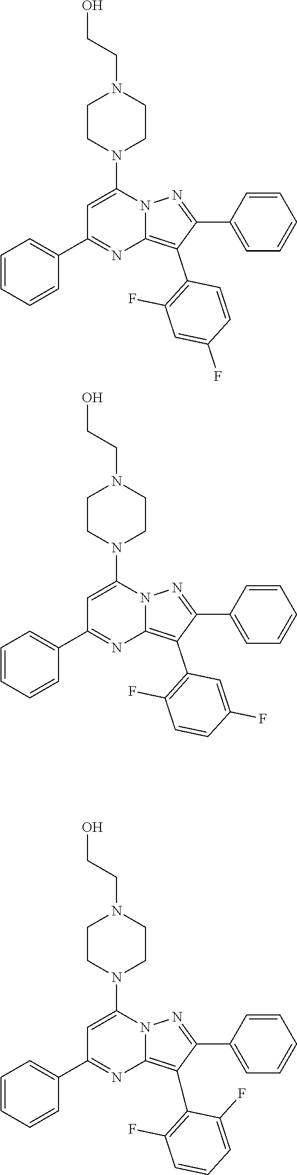 [SMILES: OCCN1CCN(c2cc(-c3ccccc3)nc3c(-c4ccc(F)cc4F)c(-c4ccccc4)nn23)CC1 ; OCCN1CCN(c2cc(-c3ccccc3)nc3c(-c4cc(F)ccc4F)c(-c4ccccc4)nn23)CC1 ; OCCN1CCN(c2cc(-c3ccccc3)nc3c(-c4c(F)cccc4F)c(-c4ccccc4)nn23)CC1]

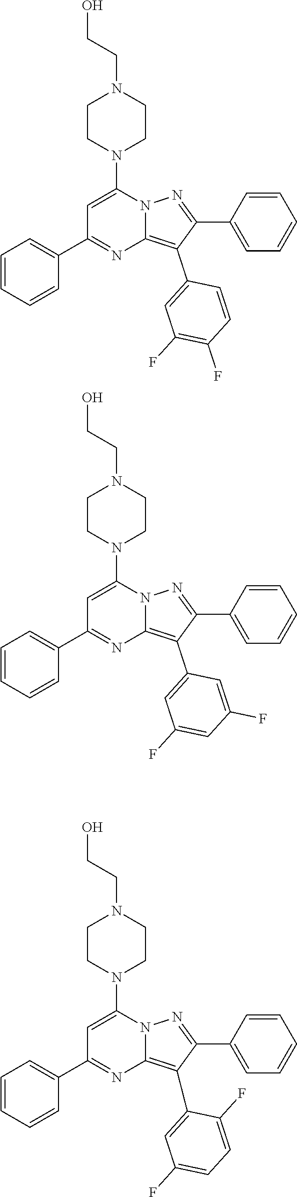 [SMILES: OCCN1CCN(c2cc(-c3ccccc3)nc3c(-c4ccc(F)c(F)c4)c(-c4ccccc4)nn23)CC1 ; OCCN1CCN(c2cc(-c3ccccc3)nc3c(-c4cc(F)cc(F)c4)c(-c4ccccc4)nn23)CC1 ; OCCN1CCN(c2cc(-c3ccccc3)nc3c(-c4cc(F)ccc4F)c(-c4ccccc4)nn23)CC1]

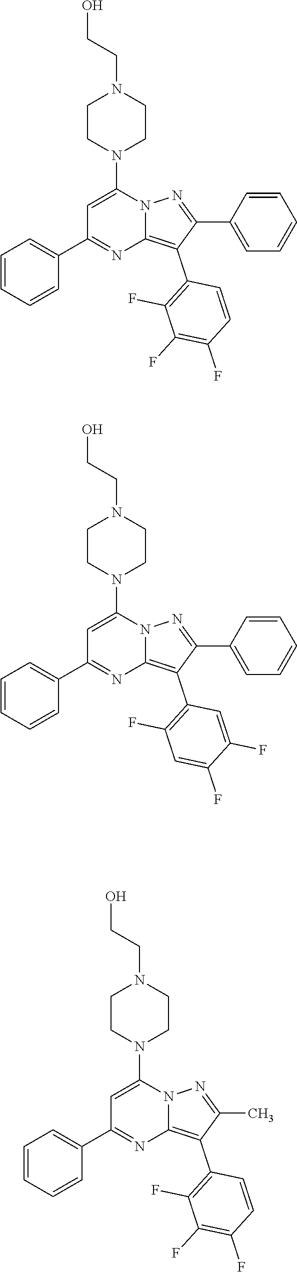 [SMILES: OCCN1CCN(c2cc(-c3ccccc3)nc3c(-c4ccc(F)c(F)c4F)c(-c4ccccc4)nn23)CC1 ; OCCN1CCN(c2cc(-c3ccccc3)nc3c(-c4cc(F)c(F)cc4F)c(-c4ccccc4)nn23)CC1 ; Cc1nn2c(N3CCN(CCO)CC3)cc(-c3ccccc3)nc2c1-c1ccc(F)c(F)c1F]

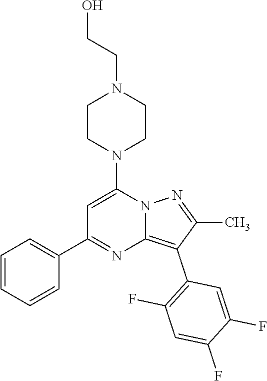 [SMILES: Cc1nn2c(N3CCN(CCO)CC3)cc(-c3ccccc3)nc2c1-c1cc(F)c(F)cc1F]

or a subgroup thereof.

In some aspects, one or both of the nitrogen atoms in the piperazine ring in the compounds disclosed herein can be protonated and/or exhibit a positive charge. In other aspects, the nitrogen atom(s) may not be charged and/or may lack an additional proton. In any of these aspects, charge state of the nitrogen will change based on solution pH, physiological pH, counter ion, solvate, salt, ester, physiological environment, and other factors known to those of skill in the art. In a further aspect, all compounds disclosed herein can take both protonated and unprotonated states at these positions; thus, depictions of these should be considered equivalent regardless of whether charge and proton are shown or not.

In various aspects, it is contemplated herein that the disclosed compounds further comprise their biosteric equivalents. The term “bioisosteric equivalent” refers to compounds or groups that possess near equal molecular shapes and volumes, approximately the same distribution of electrons, and which exhibit similar physical and biological properties. Examples of such equivalents are: (i) fluorine vs. hydrogen, (ii) oxo vs. thia, (iii) hydroxyl vs. amide, (iv) carbonyl vs, oxime: (v) carboxylate vs. tetrazole. Examples of such bioisosteric replacements can be found in the literature and examples of such are: ii) Burger A, Relation of chemical structure and biological activity: in Medicinal Chemistry Third ed., Burger A, ed.:, Wiley-Interscience; New York, 970, 64-80; iii) Burger, A.; “Isosterism and bioisosterism in drug design”; Prog. Drug Res. 1991, 37, 287-371, (iii) Burger A. “Isosterism and bioanalogy in drug design”, Mae Chem. Res, 1994, 4, 89-92; (iv) Clark R D, Ferguson A M, Cramer R D “Bioisosterism and molecular diversity”, Perspect. Drug Discovery Des. 1998, 9/10/11, 213-224, (v) Koyanagi T, Haga T, “Bioisosterism in agrochemicals”, ACS Symp. Ser. 1995, 584, 15-24; (vi) Kubinyi 1, ‘Molecular similarities. Part 1. Chemical structure and biological activity Pharm, Unserer Zeit 1998, 27, 92-106; (Vii) Lipinski C A’: “Bioisosterism in drug design”; Annu. Rep. Med. Chem. 1986, 21, 283-91′, (viii) Patani G A, LaVoie E d, “Bioisosterism: A rational approach in drug design”, Chem. Rev, (Washing n, D 1996, 96, 3147-3176; (ix) Soskic V. Joksimovic J, “Bioisosteric approach in the design of new dopaminergic/serotonergic ligands”, Curr. Med. Chem. 1998, 5, 493-512 (x) Thornber C W, “Isosterism and molecular modification in drug design”, Chem. Soc. Rev. 1979, 8, 563-80.

In a further aspect, the disclosed compounds can be isolated as solvates and, in particular, as hydrates of a disclosed compound, which can be obtained, for example, by crystallization from a solvent or from aqueous solution. In this connection, one, two, three or any arbitrary number of solvate or water molecules can combine with the compounds according to the invention to form solvates and hydrates.

The disclosed compounds can be used in the form of salts derived from inorganic or organic acids, Pharmaceutically acceptable salts include salts of acidic or basic groups present in the disclosed compounds. Suitable pharmaceutically acceptable salts include base addition salts, including alkali metal salts, e.g., sodium or potassium salts; alkaline earth metal salts, e.g., calcium or magnesium salts, and salts formed with suitable organic ligands, e.g., quaternary ammonium salts, which may be similarly prepared by reacting the drug compound with a suitable pharmaceutically acceptable base. The salts can be prepared in situ during the final isolation and purification of the compounds of the present disclosure; or following final isolation by reacting a free base function, such as a secondary or tertiary amine, of a disclosed compound with a suitable inorganic or organic acid; or reacting a free acid function, such as a carboxylic acid, of a disclosed compound with a suitable inorganic or organic base.

Acidic addition salts can be prepared in situ during the final isolation and purification of a disclosed compound, or separately by reacting moieties comprising one or more nitrogen groups with a suitable acid. In various aspects, acids which may be employed to form pharmaceutically acceptable acid addition salts include such inorganic acids as hydrochloric acid, sulfuric acid and phosphoric acid and such organic acids as oxalic acid, maleic acid, succinic acid and citric acid. In a further aspect, salts further include, but are not limited, to the following: hydrochloride, hydrobromide, hydroiodide, nitrate, sulfate, bisulfate, phosphate, acid phosphate, isonicotinate, acetate, lactate, salicylate, citrate, tartrate, pantothenate, bitartrate, ascorbate, succinate, maleate, gentisinate, fumarate, gluconate, glucuronate, saccharate, formate, benzoate, glutamate, methanesulfonate, ethanesulfonate, benzenesulfonate, p-toluenesulfonate, butyrate, camphorate, camphorsulfonate, digluconate, glycerophosphate, hemisulfate, heptanoate, hexanoate, fumarate, hydrochloride, 2-hydroxyethanesulfonate (isethionate), nicotinate: 2-naphthalenesulfonate, oxalate, pectinate, persulfate, 3-phenylpropionate, picrate, pivalate, propionate, succinate, tartrate, thiocyanate, phosphate, glutamate, bicarbonate undecanoate, and pamoate (i.e., 1,1′-methylene-bis-(2-hydroxy-3-naphthoate)) salts. Also, basic nitrogen-containing groups can be quatemized with such agents as lower alkyl halides, such as methyl, ethyl, propyl, and butyl chloride, bromides, and iodides: dialkyl sulfates like dimethyl, diethyl, dibutyl, and diamyl sulfates, long chain halides such as decyl, lauryl, myristyl and stearyl chlorides, bromides and iodides, aralkyl halides like benzyl and phenethyl bromides, and others.

Basic addition salts can be prepared in situ during the final isolation and purification of a disclosed compound, or separately by reacting carboxylic acid moieties with a suitable base such as the hydroxide, carbonate or bicarbonate of a pharmaceutical acceptable metal cation or with ammonia, or an organic primary, secondary or tertiary amine. Pharmaceutical acceptable salts include, but are not limited to, cations based on the alkali and alkaline earth metals, such as sodium, lithium, potassium, calcium, magnesium, aluminum salts and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. Other representative organic amines useful for the formation of base addition salts include diethylamine, ethylenediamine, ethanolamine, diethanolamine, piperazine and the like. In further aspects, bases which may be used in the preparation of pharmaceutically acceptable salts include the following: ammonia, L-arginine, benethamine, benzathine, calcium hydroxide, choline, deanol, diethanolamine, diethylamine, 2-(diethylamino)-ethanol, ethanolamine, ethylenediamine, N-methyl-glucamine, hydrabamine, L-lysine, magnesium hydroxide, 4-(2-hydroxyethyl)-morpholine, piperazine, potassium hydroxide, 1-(2-hydroxyethyl-pyrrolidine, secondary amine, sodium hydroxide, triethanolamine, tromethamine and zinc hydroxide.

Methods of Making the Compounds

In one aspect, C1 can be synthesized by the reaction depicted in Scheme 1:

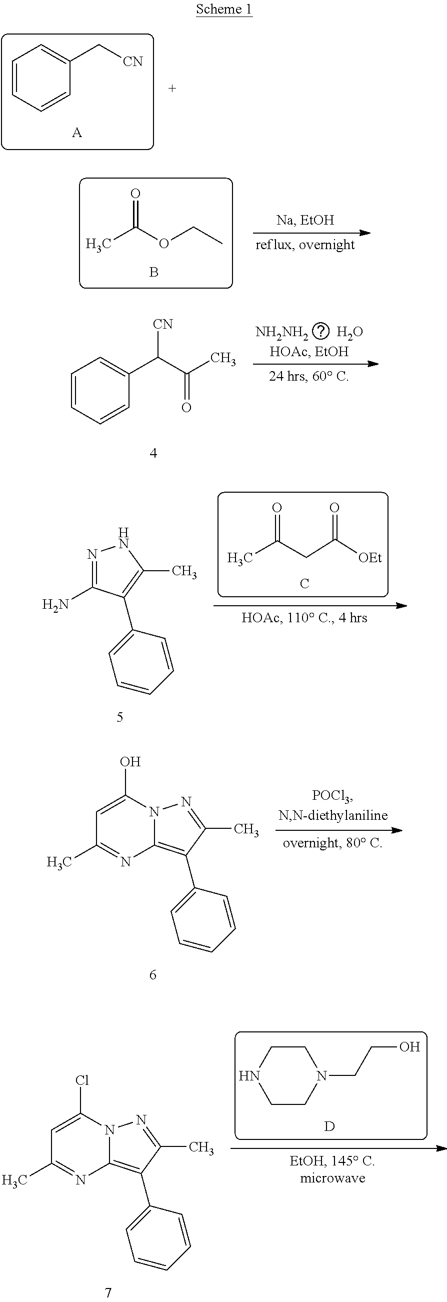

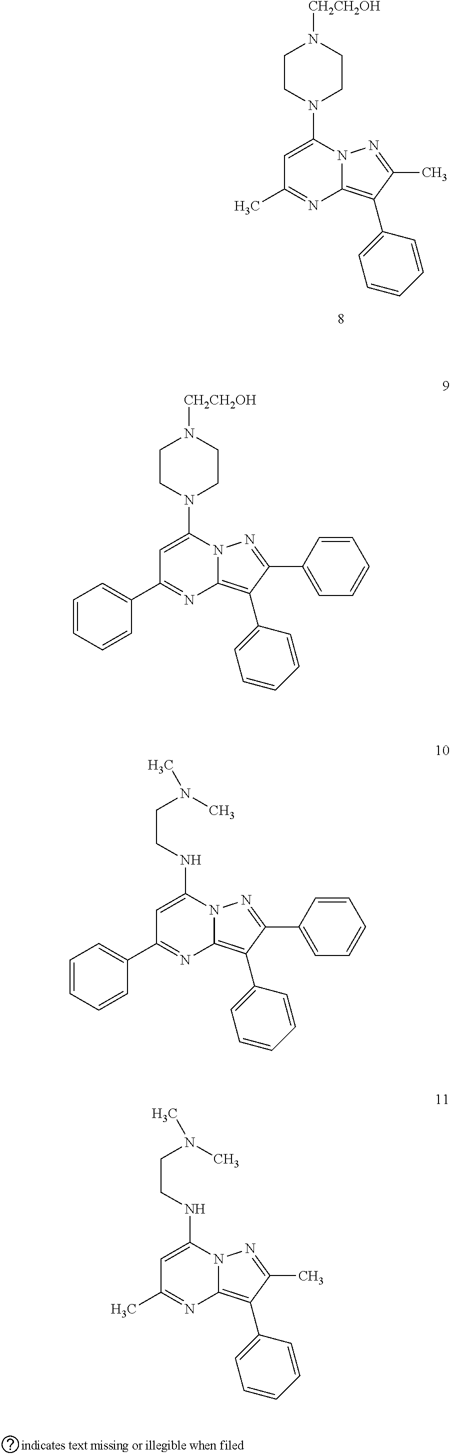

In some aspects, a nitrile (A) is first reacted with an ethyl ester (B). Other nitriles and ethyl esters in addition to those represented by (A) and (B) are also envisioned. The resulting α-cyanoketone (4) product can be treated with hydrazine to form 5-amino-3-methyl-4-phenylpyrazole (5) In one aspect, reaction with selected 1,3-ketoesters provides (6). Numerous 1,3-ketoesters are commercially available and are considered suitable for use in the reactions of Scheme 1. In a further aspect, treatment with POCl₃ results in a chloride that can undergo substitution by a wide variety of amines. Non-limiting examples are provided in Scheme 1 with the compounds represented by (8), (9), (10), and (11).

Additional reagents useful herein are depicted in Scheme 2;

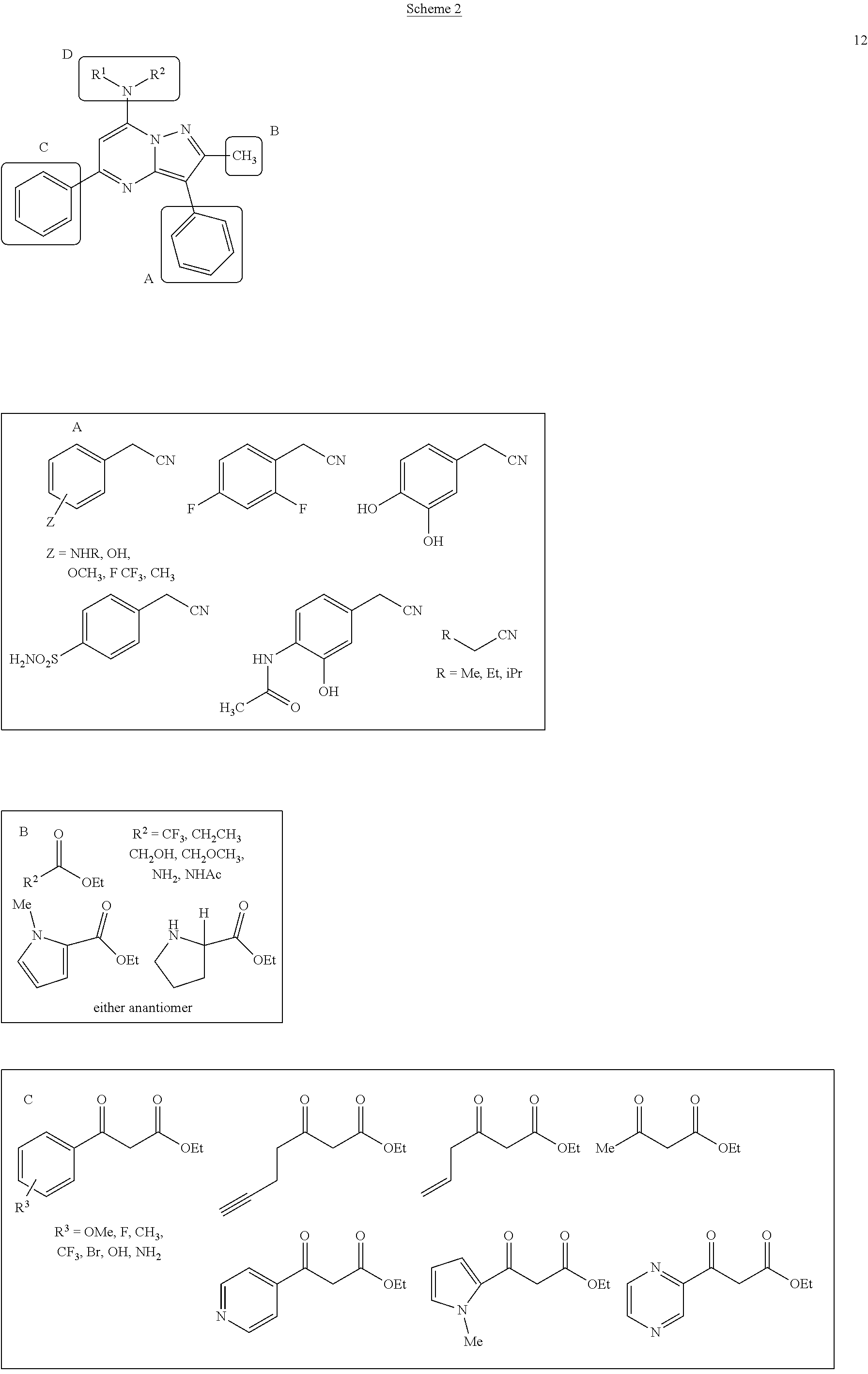

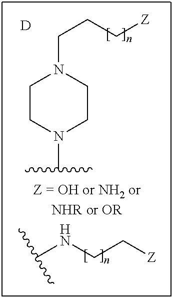

In one aspect, without wishing to be bound by theory, a met group at position C in Scheme 2 is less active, presumably due to the lack of an important TT-stacking interaction between position C and KRAS. In one aspect versions of the molecule with electron withdrawing groups attached to the benzene ring at position C in Scheme 2 should enhance the TT-stacking interaction. In a further aspect, —F and CF₃ are expected to be particularly useful. In another aspect, this interaction can be further probed by synthesizing analogs with electron-donating groups, heterocycles, and other substituents at this position. In still another aspect, alkyl groups offering potential TT-interactions, including but not limited to, alkynes and alkenes can also be substituted at position C.

In a further aspect, without wishing to be bound by theory, docking studies indicate that the methyl group in Scheme 2 at position B falls into the space between an IIe and a Leu residue, indicating that a small group may be most effective at this location. Analogs with larger groups or similarly-sized groups having different electronic properties such as, for example, —CF₃, can be used to probe this interaction.

In a still further aspect, the phenyl group at position A of Scheme 2 appears to slide into a groove with no significant interactions. This can be probed by, in some aspects, preparing molecules where site A has been substituted with electron withdrawing and/or electron donating groups can be examined, in some aspects, this site may tolerate substituents or groups on one side of the phenyl ring. In any of these aspects, substituents can be introduced to the phenyl ring to improve solubility.

in still another aspect, substitution at site D of Scheme 12 can be explored. Without wishing to be bound by theory, site D is thought to be critical to blocking the KRAS:Raf interaction. In one aspect, alkyl groups, heterocycles, alkyl groups that possess polar substituents, and the like, can be attached at position D. It is expected that substitution at site may also affect solubility of analog compounds.

Additional exemplary procedures for synthesizing and characterizing the molecules disclosed herein are provided in the Examples.

Pharmaceutical Compositions

In various aspects, the present disclosure relates to pharmaceutical compositions comprising a therapeutically effective amount of at least one disclosed compound, at least one product of a disclosed method, or a pharmaceutically acceptable salt thereof. As used herein, “pharmaceutically-acceptable carriers” means one or more of a pharmaceutically acceptable diluents, preservatives, antioxidants, solubilizers, emulsifiers, coloring agents, releasing agents, coating agents, sweetening, flavoring and perfuming agents, and adjuvants. The disclosed pharmaceutical compositions can be conveniently presented in unit dosage form and prepared by any of the methods well known in the art of pharmacy and pharmaceutical sciences.

In a further aspect, the disclosed pharmaceutical compositions comprise a therapeutically effective amount of at least one disclosed compound, at least one product of a disclosed method, or a pharmaceutically acceptable salt thereof as an active ingredient, a pharmaceutically acceptable carrier, optionally one or more other therapeutic agent, and optionally one or more adjuvant. The disclosed pharmaceutical compositions include those suitable for oral, rectal, topical, pulmonary, nasal, and parenteral administration, although the most suitable route in any given case will depend on the particular host, and nature and severity of the conditions for which the active ingredient is being administered. In a further aspect, the disclosed pharmaceutical composition can be formulated to allow administration orally, nasally, via inhalation, parenterally, paracancerally, transmucosally, transdermally, intramuscularly, intravenously, intradermally, subcutaneously, intraperitoneally, intraventricularly, intracranially and intratumorally.

As used herein, “parenteral administration” includes administration by bolus injection or infusion, as well as administration by intravenous, intramuscular, intraarterial, intrathecal, intracapsular, intraorbital, intracardiac, intradermal, intraperitoneal, transtracheal, subcutaneous, subcuticular, intraarticular, subcapsular subarachnoid, intraspinal, epidural and intrasternal injection and infusion.

In various aspects, the present disclosure also relates to a pharmaceutical composition comprising a pharmaceutically acceptable carrier or diluent and, as active ingredient, a therapeutically effective amount of a disclosed compound, a product of a disclosed method of making, a pharmaceutically acceptable salt, a hydrate thereof, a solvate thereof or a stereochemically isomeric form thereof. In a further aspect, a disclosed compound, a product of a disclosed method of making, a pharmaceutically acceptable salt, a hydrate thereof, a solvate thereof, or a stereochemically isomeric form thereof, or any subgroup or combination thereof may be formulated into various pharmaceutical forms for administration purposes.

Pharmaceutically acceptable salts can be prepared from pharmaceutically acceptable non-toxic bases or acids. For therapeutic use, salts of the disclosed compounds are those wherein the counter ion is pharmaceutically acceptable. However, salts of acids and bases which are non-pharmaceutically acceptable may also find use, for example, in the preparation or purification of a pharmaceutically acceptable compound. All salts, whether pharmaceutically acceptable or not, are contemplated by the present disclosure. Pharmaceutically acceptable acid and base addition salts are meant to comprise the therapeutically active non-toxic acid and base addition salt forms which the disclosed compounds are able to form.

In various aspects, a disclosed compound comprising an acidic group or moiety, e.g., a carboxylic acid group, can be used to prepare a pharmaceutically acceptable salt. For example, such a disclosed compound may comprise an isolation step comprising treatment with a suitable inorganic or organic base, in some cases, it may be desirable in practice to initially isolate a compound from the reaction mixture as a pharmaceutically unacceptable salt and then simply convert the latter back to the free acid compound by treatment with an acidic reagent, and subsequently convert the free acid to a pharmaceutically acceptable base addition salt. These base addition salts can be readily prepared using conventional techniques, e.g., by treating the corresponding acidic compounds with an aqueous solution containing the desired pharmacologically acceptable cations and then evaporating the resulting solution to dryness, preferably under reduced pressure. Alternatively, they also can be prepared by mixing lower alkanolic solutions of the acidic compounds and the desired alkali metal alkoxide together, and then evaporating the resulting solution to dryness in the same manner as before.

Bases which can be used to prepare the pharmaceutically acceptable base-addition salts of the base compounds are those which can form non-toxic base-addition salts, i.e., salts containing pharmacologically acceptable cations such as, alkali metal cations (e.g., lithium, potassium and sodium), alkaline earth metal cations (e.g., calcium and magnesium), ammonium or other water-soluble amine addition salts such as N-methylglucamine-(meglumine), lower alkanolammonium and other such bases of organic amines. In a further aspect, derived from pharmaceutically acceptable organic non-toxic bases include primary, secondary, and tertiary amines, as well as cyclic amines and substituted amines such as naturally occurring and synthesized substituted amines. In various aspects, such pharmaceutically acceptable organic non-toxic bases include, but are not limited to, ammonia, methylamine, ethylamine, propylamine, isopropylamine, any of the four butylamine isomers, betaine, caffeine, choline, dimethylamine, diethylamine, diethanolamine, dipropylamine, diisopropylamine, di-n-butylamine, N,N′-dibenzylethylenediamine, pyrrolidine, piperidine, morpholine, trimethylamine, thethylamine, tripropylamine, tromethamine, 2-diethylaminoethanol, 2-dimethylaminoethanol, ethanolamine, quinuclidine: pyridine, quinoline and isoquinoline; benzathine, N-methyl-D-glucamine, ethylenediamine, N-ethylmorpholine, N-ethylpiperidine, glutamine, glucosamine, methylglucamine, morpholine, piperazine, piperidine, polyamine resins, procaine, purines, theobromine, hydrabamine salts, and salts with amino acids such as, for example, histidine, arginine, lysine and the like. The foregoing salt forms can be converted by treatment with acid back into the free acid form.

In various aspects, a disclosed compound comprising a protonatable group or moiety, e.g., an amino group, can be used to prepare a pharmaceutically acceptable salt. For example, such a disclosed compound may comprise an isolation step comprising treatment with a suitable inorganic or organic acid. In some cases, it may be desirable in practice to initially isolate a compound from the reaction mixture as a pharmaceutically unacceptable salt and then simply convert the latter back to the free base compound by treatment with a basic reagent, and subsequently convert the free base to a pharmaceutically acceptable acid addition salt. These acid addition salts can be readily prepared using conventional techniques, e.g., by treating the corresponding basic compounds with an aqueous solution containing the desired pharmacologically acceptable anions and then evaporating the resulting solution to dryness, preferably under reduced pressure. Alternatively, they also can be prepared by treating the free base form of the disclosed compound with a suitable pharmaceutically acceptable non-toxic inorganic or organic acid.

Acids which can be used to prepare the pharmaceutically acceptable acid-addition setts of the base compounds are those which can form non-toxic acid-addition salts, i.e., salts containing pharmacologically acceptable anions formed from their corresponding inorganic and organic acids. Exemplary, but non-limiting, inorganic acids include hydrochloric hydrobromic, sulfuric, nitric, phosphoric and the like. Exemplary, but non-limiting, organic acids include acetic, benzenesulfonic, benzoic, camphorsulfonic, citric, ethanesulfonic, fumaric, gluconic, glutamic, isethionic, lactic, maleic, malic, mandelicmethanesulfonic, mucic, pamoic, pantothenic, succinic, tartaric, p-toluenesulfonic acid and the like. In a further aspect, the acid-addition salt comprises an anion formed from hydrobromic, hydrochloric, maleic, phosphoric, sulfuric, and tartaric acids.

In practice, the compounds of the present disclosure, or pharmaceutically acceptable salts thereof, of the present disclosure can be combined as the active ingredient in intimate admixture with a pharmaceutical carrier according to conventional pharmaceutical compounding techniques. The carrier can take a wide variety of forms depending on the form of preparation desired for administration, e.g., oral or parenteral (including intravenous). Thus, the pharmaceutical compositions of the present disclosure can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion or as a water-in-oil liquid emulsion, in addition to the common dosage forms set out above, the compounds of the present disclosure, and/or pharmaceutically acceptable salt(s) thereof, can also be administered by controlled release means and/or delivery devices. The compositions can be, prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

It is especially advantageous to formulate the aforementioned pharmaceutical compositions in unit dosage form for ease of administration and uniformity of dosage. The term “unit dosage form,” as used herein, refers to physically discrete units suitable as unitary dosages, each unit containing a predetermined quantity of active ingredient calculated to produce the desired therapeutic effect in association, with the required pharmaceutical carrier, That is, a “unit dosage form” is taken to mean a single dose wherein ail active and inactive ingredients are combined in a suitable system, such that the patient or person administering the drug to the patient can open a single container or package with the entire dose contained therein, and does not have to mix any components together from two or more containers or packages. Typical examples of unit dosage forms are tablets (including scored or coated tablets), capsules or pills for oral administration; single dose vials for injectable solutions or suspension; suppositories for rectal administration; powder packets; wafers; and segregated multiples thereof. This list of unit dosage forms is not intended to be limiting in any way, but merely to represent typical examples of unit dosage forms.

The pharmaceutical compositions disclosed herein comprise a compound of the present disclosure (or pharmaceutically acceptable salts thereof) s an active ingredient, a pharmaceutically acceptable carrier, and optionally one or more additional therapeutic agents. In various, aspects, the disclosed pharmaceutical compositions can include a pharmaceutically acceptable carrier and a disclosed compound, or a pharmaceutically acceptable salt thereof. In a further aspect, a disclosed compound, or pharmaceutically acceptable salt thereof, can also be included in a pharmaceutical composition in combination with one or more other therapeutically active compounds. The instant compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions can be conveniently presented in unit dosage form and prepared by any of the methods well known in the art of pharmacy.

Techniques and compositions for making dosage forms useful for materials and methods described herein are described, for example, in the following references: Modern Pharmaceutics, Chapters 9 and 10 (Banker & Rhodes, Editors. 1979); Pharmaceutical Dosage Forms: Tablets (Lieberman et al., 1981); Ansel, introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol 7. (David Ganderton, Trevor Jones, James MoGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modern Pharmamutics Drugs and the Pharmaceutical Sciences, Vol 40 (Gilbert S. Banker, Christopher T, Rhodes, Eds.).

The compounds described herein are typically to be administered in admixture with suitable pharmaceutical diluents, excipients, extenders, or carriers (termed herein as a pharmaceutically acceptable, carrier, or a carrier) suitably selected with respect to the intended form of administration and as consistent with conventional pharmaceutical practices. The deliverable compound will be in a form suitable for oral, rectal, topical, intravenous injection or parenteral administration. Carriers include solids or liquids, and the type of carrier is chosen based on the type of administration being used. The compounds may be administered as a dosage that has a known quantity of the compound.

Because of the ease in administration, oral administration can be a preferred dosage form, and tablets and capsules represent the most advantageous oral dosage unit forms in which, case solid pharmaceutical carriers are obviously employed. However, other dosage forms may be suitable depending upon clinical population (e.g., age and severity of clinical condition), solubility properties of the specific disclosed compound used, and the like. Accordingly, the disclosed compounds can be used in oral dosage forms such as pills, powders, granules, elixirs, tinctures, suspensions, syrups, and emulsions. In preparing the compositions for oral dosage form, any convenient pharmaceutical media can be employed. For example, water, glycols, oils, alcohols, flavoring agents, preservatives, coloring agents and the like can be used to form oral liquid preparations such as suspensions, elixirs and solutions; while carriers such as starches, sugars, microcrystalline cellulose, diluents, granulating agents, lubricants, binders, disintegrating agents, and the like can be used to form oral solid preparations such as powders, capsules and tablets. Because of their ease of administration, tablets and capsules are the preferred oral dosage units whereby solid pharmaceutical carriers are employed. Optionally, tablets can be coated by standard aqueous or nonaqueous techniques.

The disclosed pharmaceutical compositions in an oral dosage form can comprise one or more pharmaceutical excipient and/or additive. Non-limiting examples of suitable excipients and additives include gelatin, natural, sugars such as raw sugar or lactose, lecithin, pectin, starches (for example corn starch or amylose), dextran, polyvinyl pyrrolidone, polyvinyl acetate, gum arabic, alginic acid, tylose, talcum, lycopodium, silica gel (for example colloidal), cellulose, cellulose derivatives (for example cellulose ethers in which the cellulose hydroxy groups are partially etherified with lower saturated aliphatic alcohols and/or lower saturated, aliphatic oxyalcohols, for example methyl oxypropyl cellulose, methyl cellulose, hydroxypropyl methyl cellulose, hydroxypropyl methyl cellulose phthalate), fatty acids as well as magnesium, calcium or aluminum salts of fatty acids with 12 to 22 carbon atoms, in particular saturated (for example stearates), emulsifiers, oils and fats, in particular vegetable (for example, peanut oil, castor oil, olive oil, sesame oil, cottonseed oil, corn oil, wheat germ oil, sunflower seed oil, cod liver oil, in each case also optionally hydrated); glycerol esters and polyglycerol esters of saturated fatty acids C₁₂H₂₄O₂ to C₁₈H₃₆O₂ and their mixtures, it being possible for the glycerol hydroxy groups to be totally or also only partly esterified (for example mono-, di- and triglycerides); pharmaceutically acceptable mono- or multivalent alcohols and polyglycols such as polyethylene glycol and derivatives thereof, esters of aliphatic saturated or unsaturated fatty acids to 22 carbon atoms, in particular 10-18 carbon atoms) with monovalent aliphatic alcohols (1 to 20 carbon atoms) or multivalent alcohols such as glycols, glycerol, diethylene glycol, pentactythritol, sorbitol, mannitol and the like, which may optionally also be etherified, esters of citric acid with primary alcohols, acetic acid, urea, benzyl benzoate, dioxolanes, glyceroformals, tetrahydrofurfutyl alcohol, polyglycol ethers with C1-C12-alcohols, dimethylacetarrilde, lactamides, lactates, ethylcarbonates, silicones (in particular medium-viscous polydimethyl siloxanes), calcium carbonate, sodium carbonate, calcium phosphate, sodium phosphate: magnesium carbonate and the like.

Other auxiliary substances useful in preparing an oral dosage form are those which cause disintegration (so-called disintegrants), such as: cross-linked polyvinyl pyrrolidone, sodium carboxymethyl starch, sodium carboxymethyl cellulose or microcrystalline cellulose. Conventional coating substances may also be used to produce the oral dosage, form. Those that may for example be considered are; polymerizates as well as copolymerizes of acrylic acid and/or methacrylic acid and/or their esters; copolymerizes of acrylic and methacrylic acid esters with a lower ammonium group content (for example EudragitR RS), copolymerizes of acrylic and methacrylic acid esters and trimethyl ammonium methacrylate (for example EudragitR RL); polyvinyl acetate; fats, oils, waxes, fatty alcohols; hydroxypropyl methyl cellulose phthalate or acetate succinate; cellulose acetate, phthalate, starch acetate phthalate as well as polyvinyl acetate phthalate, carboxy methyl cellulose; methyl cellulose phthalate, methyl cellulose succinate, -phthalate succinate as well as methyl cellulose phthalic acid half ester; zein; ethyl cellulose as well as ethyl cellulose succinate; shellac, gluten; ethylcarboxyethyl cellulose; ethacrylate-maleic acid anhydride copolymer; maleic acid anhydride-vinyl methyl ether copolymer; styrol-maleic acid copolymerizate; 2-ethyl-hexyl-acrylate maleic acid anhydride; crotonic acid-vinyl acetate copolymer; glutaminic acid/glutamic acid ester, copolymer; carboxymethylethylcellulose glycerol monooctanoate; cellulose acetate succinate; polyarginine.

Plasticizing agents that may be considered as coating substances in the disclosed oral dosage forms are: citric and tartaric acid esters (acetyl-triethyl citrate, acetyl tributyl-, tributyl-triethyl-citrate); glycerol and glycerol esters (glycerol diacetate, -triacetate, acetylated monoglycerides, castor oil); phthalic acid esters (dibutyl-, diamyl-, diethyl-, dimethyl-, dipropyl-phthalate), di-(2-methoxy- or 2-ethoxyethylyphthalate, ethylphthalyl glycolate, butylphthalylethyl glycolate and butylglycolate; alcohols (propylene, glycol, polyethylene glycol of various chain lengths), adipates (diethyladipate, di-(2-methoxy- or 2-ethoxyethyl)-adipate; benzophenone; diethyl- and dibuqlsebacate, dibutylsuccinate, dibutyltartrate; diethylene glycol dipropionate; ethyleneglycol diacetate, -dibutyrate, -dipropionate; tributyl phosphate, tributyrin; polyethylene glycol sorbitan monooleate (polysorbates such as Polysorbar 50); sorbitan monooleate.

Moreover, suitable binders, lubricants, disintegrating agents, coloring agents, flavoring agents, flow-inducing agents, and melting agents may be included as carriers. The pharmaceutical carrier employed can be, for example, a solid, liquid, or gas. Examples of solid carriers include, but are not limited to, lactose, terra alba, sucrose, glucose, methylcellulose, dicalcium phosphate, calcium sulfate, mannitol, sorbitol talc, starch, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

In various aspects, a binder can include, for example, starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. In a further aspect, a disintegrator can include, for example, starch, methyl cellulose, agar, bentonite, xanthan gum, and the like.

In various aspects, an oral dosage form, such as a solid dosage can comprise a disclosed compound that is attached to polymers as targetable drug carriers or as a prodrug. Suitable biodegradable polymers useful in achieving controlled release of a drug include, for example, polylactic acid, polyglycolic acid, copolymers of polylactic and polyglycolic acid, caprolactones, polyhydroxy butyric acid, polyorthoesters, polyacetals, polydihydropyrans, polycyanoacylates, and hydrogels, preferably covalently crosslinked hydrogels.

Tablets may contain the active ingredient in admixture with non-toxic pharmaceutically acceptable excipients which are suitable for the manufacture of tablets. These excipients may be, for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques to delay disintegration and absorption in the gastrointestinal tract and thereby provide a sustained action over a longer period.

A tablet containing a disclosed compound can be prepared by compression or molding, optionally with one or more accessory ingredients or adjuvants. Compressed tablets can be prepared by compressing, in a suitable machine, the active ingredient in a free-flowing form such as powder or granules, optionally mixed with a binder, lubricant inert diluent, surface active or dispersing agent. Molded tablets can be made by molding in a suitable machine, a mixture of the powdered compound moistened with an inert liquid diluent.

In various aspects, a solid oral dosage form, such as a tablet, can be coated with an enteric coating to prevent ready decomposition in the stomach. In various aspects, enteric coating agents include, but are not limited to, hydroxypropylmethylcellulose phthalate, methacrylic acid-methacrylic acid ester copolymer, polyvinyl acetate-phthalate and cellulose acetate phthalate. Akihiko Hasegawa “Application of solid dispersions of Nifedipine with enteric coating agent to prepare a sustained-release dosage form” Chem. Pharm, Bull. 33:1615-1619 (1985). Various enteric coating materials may be selected on the basis of testing to achieve an enteric coated dosage form designed ab initio to have a preferable combination of dissolution time, coating thicknesses and diametral crushing strength (e.g., see S. C. Porter et at “The Properties of Enteric Tablet Coatings Made From Polyvinyl Acetate-phthalate and Cellulose acetate Phthalate”, J. Pharm. Pharmacol, 22:42p (1970)). In a further aspect, the enteric coating may comprise hydroxypropyl-methylcellulose phthalate, methacrylic acid-methacrylic acid ester copolymer, polyvinyl acetate-phthalate and cellulose acetate phthalate.

In various aspects, an, oral dosage form can be a solid dispersion with a water soluble or a water insoluble carrier. Examples of water soluble or water insoluble carrier include, but are not limited to, polyethylene glycol, polyvinylpyrrolidone, hydroxypropylmethyl-cellulose, phosphatidylcholine, polyoxyethylene hydrogenated castor oil, hydroxypropyl-methylcellulose phthalate, carboxymethylethylcellulose, or hydroxypropylmethylcellulose, ethyl cellulose, or stearic acid.

In various aspects, are oral dosage form can be in a liquid dosage form, including those that are ingested, or alternatively, administered as a mouth wash or gargle. For example, a liquid dosage form can include aqueous suspensions, which contain the active materials in admixture with excipients suitable for the manufacture of aqueous suspensions. In addition, oily suspensions may be formulated by suspending the active ingredient in a vegetable oil, for example rachis oil, olive oil, sesame oil or coconut oil, or in a mineral oil such as liquid paraffin. Oily suspensions may also contain various excipients. The pharmaceutical compositions of the present disclosure may also be in the form of oil-in-water emulsions, which may also contain excipients such as sweetening and flavoring agents.

For the preparation of solutions or suspensions it is, for example, possible to use water, particularly sterile water, or physiologically acceptable organic solvents, such as alcohols (ethanol, propanol, isopropanol, 1,2-propylene glycol, polyglycols and their derivatives, fatty alcohols, partial esters of glycerol), oils (for example peanut oil, olive oil, sesame oil, almond oil, sunflower oil, soya bean oil, castor oil, bovine hoof oil), paraffins, dimethyl sulphoxide, triglycerides and the like.

In the case of a liquid dosage form such as a drinkable solutions, the following substances may be used as stabilizers or solubilizers lower aliphatic mono- and multivalent alcohols with 2-4 carbon atoms, such as ethanol, n-propanol, glycerol, polyethylene glycols with molecular weights between 200-600 (for example 1 to 40% aqueous solution), diethylene glycol monoethyl ether, 1,2-propylene glycol, organic amides, for example amides of aliphatic C1-O6-carboxylic acids with ammonia or primary, secondary or tertiary C1-C4-amines or C1-C4-hydroxy amines such as urea, urethane, acetamide, N-methyl acetamide, N,N-diethyl acetamide, N,N-dimethyl acetamide, lower aliphatic amines and diamines with 2-6 carbon atoms, such as ethylene diamine, hydroxyethyl theophylline, tromethamine (for example as 0.1 to 20% aqueous solution), aliphatic amino acids.

In preparing, the disclosed liquid dosage form can comprise solubilizers and emulsifiers such as the following non-limiting examples can be used: polyvinyl pyrrolidone, sorbitan fatty acid esters such as sorbitan trioleate, phosphatides such as lecithin, acacia, tragacanth, polyoxyethylated sorbitan monooleate and other ethoxylated fatty acid esters of sorbitan, polyoxyethylated fats, polyoxyethylated oleotriglycerides, linolizated oleotriglycerides, polyethylene oxide condensation products of fatty alcohols, alkylphenols or fatty acids or also 1-methyl-3-(2-hydroxyethyl)imidazolidone-(2). In this context, polyoxyethylated means that the substances in question contain polyoxyethylene chains, the degree of polymerization of which generally lies between 2 and 40 and in particular between 10 and 20, Polyoxyethylated substances of this kind may for example be obtained by reaction of hydroxyl group-containing compounds (for example mono- or diglycerides or unsaturated compounds such as those containing oleic acid radicals) with ethylene oxide (for example 40 Mot ethylene oxide per 1 Mol glyceride). Examples of oleotriglycerides are olive oil, peanut oil, castor oil, sesame oil, cottonseed oil, corn oil. See also Dr. H. P, Fiedler “Lexikon der Hillsstoffe für Pharmazie, Kostnetik und angrenzende Gebiete” 1971, pages 191-195.

In various aspects, a liquid dosage form can further comprise preservatives, stabilizers, buffer substances, flavor correcting agents, sweeteners, colorants, antioxidants and complex formers and the like. Complex formers which may be for example be considered are: chelate formers such as ethylene diamine retrascetic acid, nitrilotriacetic acid, diethylene triamine pentacetic acid and their salts.

It may optionally be necessary to stabilize a liquid dosage form with physiologically acceptable bases or buffers to a pH range of approximately 6 to 9. Preference may be given to as neutral or weakly basic a pH value as possible (up to pH 8).

In order to enhance the solubility and/or the stability of a disclosed compound in a disclosed liquid dosage form, a parenteral injection form, or an intravenous injectable form, it can be advantageous to employ α-, or β-cyclodextrins or their derivatives, in particular hydroxyalkyl substituted cyclodextrins, e.g. 2-hydroxypropyl-β-cycloclextrin or sulfobutyl cyclodextrin. Also co-solvents such as alcohols may improve the solubility and/or the stability of the compounds according to the present disclosure in pharmaceutical compositions.

In various aspects, a disclosed liquid dosage form, a parenteral injection form, or an intravenous injectable form can further comprise liposome delivery systems, such as small unilamellar vesicles, large unilamellar vesicles, and multilamellar vesicles. Liposomes can be formed from a variety of phospholipids, such as cholesterol, stearylamine, or phosphatidylcholines.

Pharmaceutical compositions of the present disclosure suitable injection, such as parenteral administration, such as intravenous, intramuscular, or subcutaneous administration. Pharmaceutical compositions for injection can be prepared as solutions or suspensions of the active compounds in water. A suitable surfactant can be included such as, for example, hydroxypropylcellulose, Dispersions can also be prepared in glycerol, liquid polyethylene glycols, and mixtures thereof in oils. Further, a preservative can be included to prevent the detrimental growth of microorganisms.

Pharmaceutical compositions of the present disclosure suitable for parenteral administration can include sterile aqueous or oleaginous solutions, suspensions, or dispersions. Furthermore, the compositions can be in the form of sterile powders for the extemporaneous preparation of such sterile injectable solutions or dispersions. In some aspects, the final injectable form is sterile and must be effectively fluid for use in a syringe. The pharmaceutical compositions should be stable under the conditions of manufacture and storage; thus, preferably should be, preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

Injectable solutions, for example, can be prepared in which the carrier comprises saline solution, glucose solution or a mixture of saline and glucose solution. Injectable suspensions may also be prepared in which case appropriate liquid carriers, suspending agents and the like may be employed. In some aspects, a disclosed Parenteral formulation can comprise about 0.01-0.1 M, e.g., about 0.05 M, phosphate buffer. In a further aspect, a disclosed parenteral formulation can comprise about 0.9% saline.

In various aspects, a disclosed parenteral pharmaceutical composition can comprise pharmaceutically acceptable carriers such as aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include but not limited to water, alcoholic/aqueous solutions, emulsions or suspensions, including saline and buffered media. Parenteral vehicles can include mannitol, normal serum albumin, sodium chloride solution, Ringer's dextrose, dextrose and sodium chloride, lactated Ringer's and fixed oils. Intravenous vehicles include fluid and nutrient replenishers, electrolyte replenishers such as those based on Ringer's dextrose, and the like. Preservatives and other additives may also be present, such as, for example, antimicrobials, antioxidants, collating agents, inert gases and the like. In a further aspect, a disclosed parenteral pharmaceutical composition can comprise may contain minor amounts of additives such as substances that enhance isotonicity and chemical stability, e.g., buffers and preservatives. Also contemplated for injectable pharmaceutical compositions are solid form preparations that are intended to be converted, shortly before use, to liquid form preparations. Furthermore, other adjuvants can be included to render the formulation isotonic with the blood of the subject or patient.

In addition to the pharmaceutical compositions described herein above, the disclosed compounds can also be formulated as a depot preparation. Such long acting formulations can be administered by implantation (e.g., subcutaneously or intramuscularly) or by intramuscular injection. Thus, for example, the compounds can be formulated with suitable polymeric or hydrophobic materials (e.g., as an emulsion in an acceptable oil) or ion exchange resins, or as sparingly soluble derivatives, e.g., as a sparingly soluble salt.

Pharmaceutical compositions of the present disclosure can be in a form suitable for topical administration. As used herein, the phrase “topical application” means administration onto a biological surface, whereby the biological surface includes, for example, a skin area (e.g., hands, forearms, elbows, legs, face, nails, anus and genital areas) or a mucosal membrane. By selecting the appropriate carrier and optionally other ingredients that can be included in the composition, as is detailed herein below, the compositions of the present invention may be formulated into any form typically employed for topical application. A topical pharmaceutical composition can be in a form of a cream: an ointment, a paste, a gel, a lotion, milk, a suspension, an aerosol, a spray, foam, a dusting powder, a pad, and a patch. Further, the compositions can be in a form suitable for use in transdermal devices. These formulations can be prepared, utilizing a compound of the present disclosure, or pharmaceutically acceptable salts thereof, via conventional processing methods. As an example, a cream or ointment is prepared by mixing hydrophilic material and water, together with about 5 wt % to about 10 wt % of the compound, to produce a cream or ointment having a desired consistency.

In the compositions suitable for percutaneous administration, the carrier optionally comprises a penetration enhancing agent and/or a suitable wetting agent, optionally combined with suitable additives of any nature in minor proportions, which additives do not introduce a significant deleterious effect on the skin. Said additives may facilitate the administration to the skin and/or may be helpful for preparing the desired compositions. These compositions may be administered in various ways, e.g.: as a transdermal patch, as a spot-on, as an ointment.

Ointments are semisolid preparations, typically based on petrolatum or petroleum derivatives. The specific ointment base to be used is one that provides for optimum delivery for the active agent chosen for a given formulation, and, preferably, provides for other desired characteristics as well (e.g., emollience). As with other carriers or vehicles, an ointment base should be inert, stable, nonirritating and nonsensitizing. As explained in Remington: The Science and Practice of Pharmacy, 19th Ed., Easton, Pa.: Mack Publishing Co. (1995), pp. 1399-1404, ointment bases may be grouped in four classes: oleaginous bases; emulsifiable bases; emulsion bases; and water-soluble bases. Oleaginous ointment bases include, for example, vegetable oils, fats obtained from animals, and semisolid hydrocarbons obtained from petroleum. Emulsifiable ointment bases: also known as absorbent ointment bases, contain little or no water and include, for example, hydroxystearin sulfate, anhydrous lanolin and hydrophilic petrolatum. Emulsion ointment bases are either water-in-oil (W/O) emulsions or oil-in-water (O/W) emulsions, and include, for example, cetyl alcohol, glyceryl monostearate, lanolin and stearic acid. Preferred water-soluble ointment bases are prepared from polyethylene glycols of varying molecular weight.

Lotions are preparations that are to be applied to the skin surface without friction. Lotions are typically or semiliquid preparations in which solid particles, including the active agent, are present in a water or alcohol base, Lotions are typically preferred for treating large body areas, due to the ease of applying a more fluid composition. Lotions are typically suspensions of solids, and oftentimes comprise a liquid oily emulsion of the oil-in-water type. It is generally necessary that the insoluble matter in a lotion be finely divided, Lotions typically contain suspending agents to produce better dispersions as well as compounds useful for localizing and holding the active agent in contact with the skin, such as methylcellulose, sodium carboxymethyl-cellulose, and the like.

Creams are viscous liquids or semisolid emulsions, either oil-in-water or water-in-oil. Cream bases are typically water-washable, and contain an oil phase, an emulsifier and an aqueous phase. The oil phase, also called the “internal” phase, is generally comprised of petrolatum and/or a fatty alcohol such as cetyl or stearyl alcohol. The aqueous phase typically, although not necessarily, exceeds the oil phase in volume, and generally contains a humectant. The emulsifier in a cream formulation is generally a nonionic, anionic, cationic or amphoteric surfactant. Reference may be made to Remington; The Science and Practice of Pharmacy, supra, for further information.

Pastes are semisolid dosage forms in which the bioactive agent is suspended in a suitable base. Depending on the nature of the base, pastes are divided between fatty pastes or those made from a single-phase aqueous gel. The base in a fatty paste is generally petrolatum, hydrophilic petrolatum and the like. The pastes made from single-phase aqueous gels generally incorporate carboxymethylcellulose or the like as a base. Additional reference may be made to Remington: The Science and Practice of Pharmacy, for further information.

Gel formulations are semisolid, suspension-type systems. Single-phase gels contain organic macromolecules distributed substantially uniformly throughout the carrier liquid, which is typically aqueous, but also, preferably, contain an alcohol and, optionally, an oil. Preferred organic macromolecules, i.e., gelling agents, are crossbred acrylic acid polymers such as the family of carbomer polymers, e.g., carboxypolyalkylenes that may be obtained commercially under the trademark Carbopol™. Other types of preferred polymers in this context are hydrophilic polymers such as polyethylene oxides, polyoxyethylene-polyoxypropylene copolymers and polyvinylalcohol; modified cellulose, such as hydroxypropyl cellulose, hydroxyethyl cellulose, hydroxypropyl methylcellulose, hydroxypropyl methylcellulose phthalate, and methyl cellulose: gums such as tragacanth and xanthan gum; sodium alginate; and gelatin. In order to prepare a uniform gel, dispersing agents such as alcohol or glycerin can be added, or the gelling agent can be dispersed by trituration, mechanical mixing or stirring, or combinations thereof.

Sprays generally provide the active agent in an aqueous and/or alcoholic solution which can be misted onto the skin for delivery. Such sprays include those formulated to provide for concentration of the active agent solution at the site of administration following delivery, e.g., the spray solution can be primarily composed of alcohol or other like volatile liquid in which the active agent can be dissolved. Upon delivery to the skin, the carrier evaporates, leaving concentrated active agent at the site of administration.

Foam compositions are typically formulated in a single or multiple phase liquid form and housed in a suitable container, optionally together with a propellant which facilitates the expulsion of the composition from the container, thus transforming it into a foam upon application. Other foam forming techniques include, for example the Bag-in-a-can formulation technique. Compositions thus formulated typically contain a low-boiling hydrocarbon, e.g., isopropane. Application and agitation of such a composition at the body temperature cause the isopropane to vaporize and generate the foam, in a manner similar to a pressurized aerosol foaming system. Foams can be water-based or aqueous alkanolic, but are typically formulated with high alcohol content which, upon application to the skin of a user, quickly evaporates, driving the active ingredient through the upper skin layers to the site of treatment.

Skin patches typically comprise a backing, to which a reservoir containing the active agent is attached. The reservoir can be, for example, a pad in which the active agent or composition is dispersed or soaked, or a liquid reservoir. Patches typically further include a frontal water permeable adhesive, which adheres and secures the device to the treated region. Silicone rubbers with self-adhesiveness can alternatively be used. In both cases, a protective permeable layer can be used to protect the adhesive side of the patch prior to its use. Skin patches may further comprise a removable cover, which serves for protecting it upon storage.

Examples of patch configuration which can be utilized with the present invention include a single-layer or multi-layer drug-in-adhesive systems which are characterized by the inclusion of the drug directly within the skin-contacting adhesive. In such a transdermal patch design, the adhesive not only serves to affix the patch to the skin, but also serves as the formulation foundation, containing the drug and all the excipients under a single backing film. In the multi-layer drug-in-adhesive patch a membrane is disposed between two distinct drug-in-adhesive layers or multiple drug-in-adhesive layers are incorporated under a single backing film.

Examples of pharmaceutically acceptable carriers that are suitable for pharmaceutical compositions for topical applications include carrier materials that are well-known for use in the cosmetic and medical arts as bases for e.g., emulsions, creams, aqueous solutions, oils, ointments, pastes, gels, lotions, milks, foams, suspensions, aerosols and the like, depending on the final form of the composition. Representative examples of suitable carriers according to the present invention therefore include, without limitation, water, liquid alcohols, liquid glycols, liquid polyalkylene glycols, liquid esters, liquid amides, liquid protein hydrolysates, liquid alkylated protein hydrolysates, liquid lanolin and lanolin derivatives, and like materials commonly employed in cosmetic and medicinal compositions. Other suitable carriers according to the present invention include, without limitation, alcohols, such as, for example, monohydric and polyhydric alcohols, e.g., ethanol, isopropanol, glycerol, sorbitol, 2-methoxyethanol, diethyleneglycol, ethylene glycol, hexyleneglycol, mannitol, and propylene glycol; ethers such as diethyl or dipropyl ether; polyethylene glycols and methoxypolyoxyethylenes (carbowaxes having molecular weight ranging from 200 to 20,000); polyoxyethylene glycerols, polyoxyethylene sorbitols, stearoyl diacetin, and the like.

Topical compositions of the present disclosure can, if desired, be presented in a pack or dispenser device, such as an FDA-approved kit, which may contain one or more unit dosage forms containing the active ingredient. The dispenser device may, for example, comprise a tube. The pack or dispenser device may be accompanied by instructions for administration. The pack or dispenser device may also be accompanied by a notice in a form prescribed by a governmental agency regulating the manufacture, use, or sale of pharmaceuticals, which notice is reflective of approval by the agency of the form of the compositions for human or veterinary administration. Such notice, for example, may include labeling approved by the U.S. Food and Drug Administration for prescription drugs or of an approved product insert. Compositions comprising the topical composition of the invention formulated in a pharmaceutically acceptable carrier may also be prepared, placed in an appropriate container and labeled for treatment of an indicated condition.

Another patch system configuration which can be used by the present invention is a reservoir transdermal system design which is characterized by the inclusion of a liquid compartment containing a drug solution or suspension separated from the release liner by a semi-permeable membrane and adhesive. The adhesive component of this patch system can either be incorporated as a continuous layer between the membrane and the release liner or in a concentric configuration around the membrane. Yet another patch system configuration which can be utilized by the present invention is a matrix system design which is characterized by the inclusion of a semisolid matrix containing a drug solution or suspension which is in direct contact with the release liner. The component responsible for skin adhesion is incorporated in an overlay and forms a concentric configuration around the semisolid matrix.

Pharmaceutical compositions of the present disclosure, can be in a form suitable for rectal administration wherein the carrier is a solid. It is preferable that the mixture forms unit dose suppositories. Suitable carriers include cocoa butter and other materials commonly used in the art. The suppositories can be conveniently formed by first admixing the composition with the softened or melted carrier(s) followed by chilling and shaping, in molds.

Pharmaceutical compositions containing a compound of the present disclosure, and/or pharmaceutically acceptable salts thereof, can also be prepared in powder or liquid concentrate form.

The pharmaceutical composition (or formulation) may be packaged in a variety of ways. Generally, an article for distribution includes a container that contains the pharmaceutical composition in an appropriate form. Suitable containers are well known to those skilled in the art and include materials such as bottles (plastic and glass), sachets, foil blister packs, and the like. The container may also include a tamper proof assemblage to prevent indiscreet access to the contents of the package. In addition, the container typically has deposited thereon a label that describes the contents of the container and any appropriate warnings or instructions.

The disclosed pharmaceutical compositions may, if desired, be presented in a pack or dispenser device which may contain one or more unit dosage forms containing the active ingredient. The pack may for example comprise metal or plastic foil, such as a blister pack. The pack, or dispenser device may be accompanied by instructions for administration. The pack or dispenser may also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, may be the labeling approved by the U.S. Food and Drug Administration for prescription drugs, or the approved product insert. Pharmaceutical compositions comprising a disclosed compound formulated in a compatible pharmaceutical carrier may also be prepared, placed in an appropriate container, and labeled for treatment of an indicated condition.

The exact dosage and frequency of administration depends on the particular disclosed compound, a product of a disclosed method of making, a pharmaceutically acceptable salt, solvate, a hydrate thereof, a solvate thereof, or a stereochemically isomeric form thereof; the particular condition being treated and the severity of the condition being treated; various factors specific to the medical history of the subject to whom the dosage is administered such as the age; weight, sex, extent of disorder and general physical condition of the particular subject, as well as other medication the individual may be taking; as is well known to those skilled in the art. Furthermore, it is evident that said effective daily amount may be lowered or increased depending on the response of the treated subject and/or depending on the evaluation of the physician prescribing the compounds of the present disclosure.

Depending on the mode of administration, the pharmaceutical composition will comprise from 0.05 to 99% by weight, preferably from 0.1 to 70% by weight, more preferably from 0.1 to 60% by weight of the active ingredient, and, from 1 to 99.95% by weight, preferably from 30 to 99.9% by weight, more preferably from 50 to 99.9% by weight of a pharmaceutically acceptable carrier, all percentages being based on the total weight of the composition.

In the treatment of conditions that require reduced KRAS activity, an appropriate dosage level will generally be about 0.01 to 1000 mg per kg patient body weight per day and can be administered in single or multiple doses. In various aspects, the dosage level be about 0.1 to about 500 mg/kg per day, about 0.1 to 250 mg/kg per day, or about 0.5 to 100 mg/kg per day. A suitable dosage level can be about 0.01 to 1000 mg/kg per day, about 0.01 to 500 mg/kg per day, about 0.01 to 250 mg/kg per day, about 0.05 to 100 mg/kg per day, or about 0.1 to 50 mg/kg per day. Within this range the dosage can be 0.05 to 0.5, 0.5 to 5.0 or 5.0 to; 50 mg/kg per day. For oral administration, the compositions are preferably provided in the form of tablets containing 1.0 to 1000 mg of the active ingredient, particularly 1.0, 5.0, 10, 15, 20, 25, 50, 75, 100, 150, 200, 250, 300, 400, 500, 600, 750, 800, 900 and 1000 mg of the active ingredient for the symptomatic adjustment of the dosage of the patient to be treated. The compound can be administered on a regimen of 1 to 4 times per day, preferably once or twice per day. This dosing regimen can be adjusted to provide the optimal therapeutic response.

Such unit doses as described hereinabove and hereinafter can be administered more than once a day, for example, 2, 3, 4, 5 or 6 times a day. In various aspects, such unit doses an be administered 1 or 2 times per day, so that the total dosage for a 70 kg adult is in the range of 0.001 to about 15 mg per kg weight of subject per administration. In a further aspect, dosage is 0.01 to about 1.5 mg per kg weight of subject per administration, and such therapy can extend for a number of weeks or months, and in some cases, years. It will be understood, however, that the specific dose level for any particular patient will depend on a variety of factors including the activity of the specific compound employed; the age, body weight, general health, sex and diet of the individual being treated; the time and route of administration; the rate of excretion; other drugs that have previously been administered; and the severity of the particular disease undergoing therapy, as is well understood by those of skill in the area.

A typical dosage can be one 1 mg to about 100 mg tablet or 1 mg to about 300 mg taken once a day, or, multiple times per day, or one time-release capsule or tablet taken once a day and containing a proportionally higher content of active ingredient. The time-release effect can be obtained by capsule materials that dissolve at different pH values, by capsules that release slowly by osmotic pressure, or by any other known means of controlled release.

It can be necessary to use dosages outside these ranges in some cases as will be apparent to those skilled in the art. Further, it is noted that the clinician or treating physician will know now and when to start, interrupt, adjust, or terminate therapy in conjunction with individual patient response.

The present disclosure is further directed to a method for the manufacture of a medicament for modulating KRAS activity (e.g., treatment of one or more cancers or other disorders associated with KRAS dysfunction) in mammals (e.g., humans) comprising combining one or more disclosed compounds, products, or compositions with a pharmaceutically acceptable carrier or diluent. Thus, in one aspect, the present disclosure further relates to a method for manufacturing a medicament comprising combining at least one disclosed compound or at least one disclosed product with a pharmaceutically acceptable carrier or diluent.

The disclosed pharmaceutical compositions can further comprise other therapeutically active compounds, which are usually applied in the treatment of the above mentioned pathological or clinical conditions.

It is understood that the disclosed compositions can be prepared from the disclosed compounds. It is also understood that the disclosed compositions can be employed in the disclosed methods of using.

As already mentioned, the present disclosure relates to a pharmaceutical composition comprising a therapeutically effective amount of a disclosed compound, a product of a disclosed method of making, a pharmaceutically acceptable salt, a hydrate thereof, a solvate thereof, and a pharmaceutically acceptable carrier. Additionally, the present disclosure relates to a process for preparing such a pharmaceutical composition, characterized in that a pharmaceutically acceptable, carrier is intimately mixed with a therapeutically effective amount of a compound according to the present disclosure.

As already mentioned, the present disclosure also relates to a pharmaceutical composition comprising a disclosed compound, a product of a disclosed method of making, a pharmaceutically acceptable salt, a hydrate thereof, a solvate thereof, and, one or more other drugs in the treatment, prevention, control, amelioration, or reduction of risk of diseases or conditions for a disclosed compound or the other drugs may have utility as well as to the use of such a composition for the manufacture of a medicament. The present disclosure also relates to a combination of disclosed compound, a product of a disclosed method of making, a pharmaceutically acceptable salt, a hydrate thereof, a solvate thereof, and a KRAS inhibitor. The present disclosure also relates to such a combination for use as a medicine. The present disclosure also relates to a product comprising (a) disclosed compound, a product of a disclosed method of making, a pharmaceutically acceptable salt, a hydrate thereof, a solvate thereof, and (b) an additional anticancer therapeutic agent, as a combined preparation for simultaneous, separate or sequential use in the treatment or prevention of a condition in a mammal, including a human, the treatment or prevention of which is affected or facilitated by the modulatory effect of the disclosed compound and the additional therapeutic agent. The different drugs of such a combination or product may be combined in a single preparation together with pharmaceutically acceptable carriers or diluents, or they may each be present in a separate preparation together with pharmaceutically acceptable carriers or diluents.

in one aspect, disclosed herein is a pharmaceutical composition including a therapeutically effective amount of any of the compounds disclosed herein or a pharmaceutically acceptable salt, solvate, and a pharmaceutically acceptable carrier.

Methods of Using the Compounds

In a further aspect, the present disclosure provides methods of treatment comprising administration of a therapeutically effective amount of a disclosed compound or pharmaceutical composition as disclosed herein above to a subject in need thereof.

The compounds, salts, esters, solvates, and the like disclosed herein can be provided to subjects in need of treatment for a cancer or other disorder associated with aberrant KRAS activity. Optimization of the structures disclosed herein for binding with KRAS can result in better patient outcomes.

C1 possesses a pyrazolopyrimidine core instead of the indole and/or imidazole cores common in previously known KRAS ligands. C1 is relatively large, with its pyrazole ring being methylated and benzylated and its pyrimidine ring modified by benzene and 1-piperazinethanol. These substituents allow C1 to make extensive predicted contacts with KRAS residues in the switch 2 region (residues 30-40) and the effector-binding loop (residues 56-75). As a result, C1 binds to GTP-KRAS with sub micromolar affinity but appears to have much weaker affinity for GDP-KRAS as well as GTP-/GDP-bound NRAS and HRAS. C1 significantly reduces effector binding, inhibits KRAS-mediated MAPK signaling, and inhibits the proliferation of cancer cells. This mechanism of action is unique given that previously known KRAS inhibitors primarily affect guanine nucleotide exchange factor (GEF) activity.

In one aspect, disclosed herein is a method for treatment of a cancer associated with KRAS dysfunction in a mammal, wherein the method includes the step of administering to the mammal a therapeutically effective amount of at least one compound disclosed herein or a pharmaceutically acceptable salt thereof. In some aspects, the mammal is a human. In a further aspect, the mammal has been diagnosed with a need for treatment of the cancer prior to the administering step. In some aspects, the method further includes the step of identifying a mammal in need of treatment of the cancer.

In any of these aspects, the cancer can be selected from colorectal cancer, small cell lung cancer, non-small cell lung cancer, pancreatic cancer, ovarian cancer, thyroid cancer, stomach cancer, squamous cell carcinoma, melanoma, bladder cancer, renal cell carcinoma, another cancer, or a combination thereof.

In still another aspect, disclosed herein is a method for the treatment of disorder of uncontrolled cellular proliferation associated with KRAS dysfunction in a mammal, the method including the step of administering to the mammal a therapeutically effective amount of any of the compounds disclosed herein or a pharmaceutically acceptable salt thereof. In one aspect, the mammal is a human. In another aspect, the mammal has been diagnosed with a need for treatment of the disorder prior to the administering step. In still another aspect, the method also includes the step of identifying a mammal in need of treatment of the disorder.

In yet another aspect, disclosed herein is a method for inhibition of aberrant KRAS activity in at least one cell, the method including the step of contacting the at least one cell with an effective amount of at least one of any of the compounds disclosed herein or a pharmaceutically acceptable salt thereof. In another aspect, the cell is mammalian. In still another aspect, the cell is human. In one aspect, the cell has been isolated from a mammal prior to the contacting step. In yet another aspect, the contacting step is via administration of the compound to a mammal. In one aspect, the mammal has been diagnosed with a need for treatment of a disorder related to aberrant KRAS activity prior to the administering step.

Kits

Also disclosed are kits comprising at least one disclosed compound, or a pharmaceutically acceptable salt, hydrate, solvate, ester, and one or more of: (a) at least one agent known to decrease KRAS activity; (b) at least one agent known to treat a disorder or a cancer associated with increased KRAS activity; (c) at least one agent known to treat a disease of uncontrolled cellular proliferation; (d) instructions for treating a disorder associated with increased KRAS activity; and/or (e) instructions for administering the compound in connection with radiation therapy or another anti-cancer therapy. When at least one compound disclosed herein and at least one agent known to treat a disease of uncontrolled cellular proliferation, the at least one compound and at least one agent can be co-formulated, in some aspects, or can be co-packaged, in some aspects.

The disclosed compounds and/or pharmaceutical compositions comprising the disclosed compounds can conveniently be presented as a kit, whereby two or more components, which may be active or inactive ingredients, carriers, diluents, and the like, are provided with instructions for preparation of the actual dosage form by the patient or person administering the drug to the patient. Such kits may be provided with all necessary materials and ingredients contained therein, or they may contain instructions for using or making materials or components that must be obtained independently by the patient or person administering the drug to the patient. In further aspects, a kit can include optional components that aid in the administration of the unit dose to patients, such as vials for reconstituting powder forms, syringes for injection, customized IV, delivery systems, inhalers, etc, Additionally, a kit can contain instructions for preparation and administration of the compositions. The kit can be manufactured as a single use unit dose for one patient, multiple uses for a particular patient (at a constant dose or in which the individual compounds may vary in potency as therapy progresses); or the kit may contain multiple doses suitable for administration to multiple patients (“bulk packaging”). The kit components may be assembled in cartons, blister packs, bottles, tubes, and the like.

In a further aspect, the disclosed kits can be packaged in a daily dosing regimen (e.g., packaged on cards, packaged with dosing cards, packaged on blisters or blow-molded plastics, etc.). Such packaging promotes products and increases patient compliance with drug regimens. Such packaging can also reduce patient confusion. The present invention also features such kits further containing instructions for use.

In a further aspect, the present disclosure also provides a pharmaceutical pack or kit comprising one or more containers filled with one or more of the ingredients of the pharmaceutical compositions of the invention, Associated with such container(s) can be a notice in the form prescribed by a governmental agency regulating the manufacture, use or sale of pharmaceuticals or biological products, which notice reflects approval by the agency of manufacture, use or sale for human administration.

In various aspects, the disclosed kits can also comprise compounds and/or products co-packaged, co-formulated, and/or co-delivered with other components. For example, a drug manufacturer, a drug reseller, a physician, a compounding shop, or a pharmacist can provide a kit comprising a disclosed compound and/or product and another component for delivery to a patient.

It is contemplated that the disclosed kits can be used in connection with the disclosed methods of making, the disclosed methods of using or treating, and/or the disclosed compositions.

Research Tools

The disclosed compounds and pharmaceutical compositions have activity as inhibitors of mutated forms of KRAS As such, the disclosed compounds are also useful as research tools. Accordingly, one aspect of the present disclosure relates to a method of using a compound of the invention as a research tool, the method comprising conducting a biological assay using a compound of the invention. Compounds of the invention can also be used to evaluate new chemical compounds. Thus another aspect of the invention relates to a method of evaluating a test compound in a biological assay, comprising: (a) conducting a biological assay with a test compound to provide a first assay value; (b) conducting, the biological assay with a compound of the invention to provide a second assay value; wherein step (a) is conducted either before, after or concurrently with step (b); and (c) comparing the first assay value from step (a) with the second assay value from step (b). Still another aspect of the invention relates to a method of studying a biological system, e.g., a model animal for a clinical condition, or biological sample comprising a cell or tissue sample expressing a mutated KRAS protein in the cell membrane, the method comprising: (a) contacting the biological system or sample with a compound of the invention; and (b) determining the effects caused by the compound on the biological system or sample.

Now having described the aspects of the present disclosure, in general, the following Examples describe some additional aspects of the present disclosure. While aspects of the present disclosure are described in connection with the following examples and the corresponding text and figures, there is no intent to limit aspects of the present disclosure to this description. On the contrary, the intent is to cover all alternatives, modifications, and equivalents included within the spirit and scope of the present disclosure.

### Examples

The following examples are put forth so as to provide those of ordinary skill in the art with a complete disclosure and description of how the compounds, compositions, articles, devices and/or methods claimed herein are made and evaluated, and are intended to be purely exemplary of the disclosure and are not intended to limit the scope of what the inventors regard as their disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1: Protocol for the Synthesis of Pyrazolopyrimidine Derivatives

The following exemplary synthetic procedure can be modified to produce additional pyrazolopyrimidine derivatives according to the present disclosure by modification of starting materials (i.e., using commercially available or synthesized reagents with, for example, different substituents).

To produce the following compound, the following synthetic procedure Baas employed:

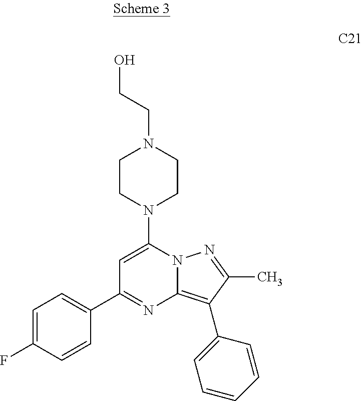 [SMILES: "C21" ; Cc1nn2c(N3CCN(CCO)CC3)cc(-c3ccc(F)cc3)nc2c1-c1ccccc1]

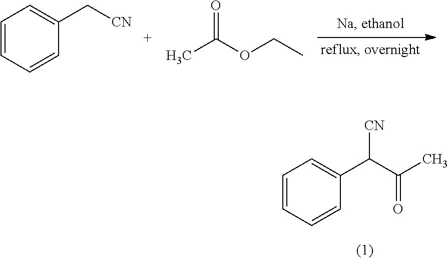

To a 100 mL three neck flask equipped with a reflux condenser and a mechanical stirrer were added, 26 mL, absolute ethanol and sodium (1200 mg, 52 mmol). The resulting mixture was stirred at room temperature until sodium completely dissolved. Phenylacetonitrile (4.8 mL, 40 mmol) was, added to the sodium ethoxide at room temperature followed by anhydrous ethyl acetate (5.8 mL, 60 mmol). The resulting mixture was refluxed overnight under nitrogen. After cooling to room temperature the resultant precipitate was dissolved in water and washed with dichloromethane. The organic layer was discarded. Then 1 M HCl was, added dropwise until the aqueous phase without any solid precipitate was obtained. The aqueous phase was extracted with DCM and the combined organic phase was washed with saturated NaCl and dried over anhydrous magnesium sulfate. The solvent was concentrated under reduced pressure. The resultant product was dried and used in next step without further purification.

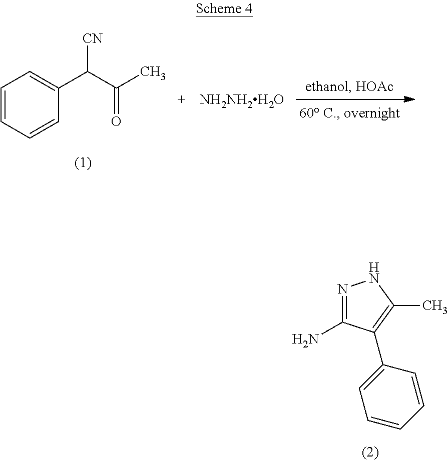

To a solution of compound 1 (2225 g, 14 mmol) in absolute ethanol (74 mL), 086 of acetic acid was added. While stirring under nitrogen at room temperature, hydrazine monohydrate was added dropwise. The reaction mixture was stirred at 60° C. overnight, After cooling to room temperature, the reaction mixture was evaporated to dryness. Then the residue was extracted with water and saturated NaHCO₃. Organic layer was washed with saturated NaCl and dried over anhydrous magnesium sulfate. The solvent was concentrated under reduced pressure. The crude material was purified by column chromatography on silica using a 10% MeOH/DCM as the solvent to obtain compound 2 in 88% yield.

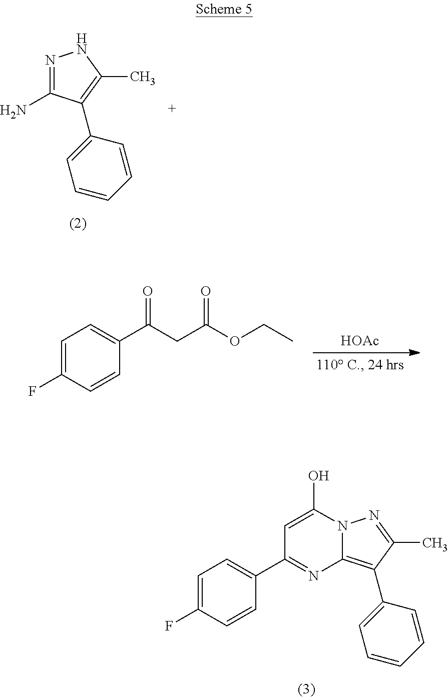 [SMILES: Cc1[nH]nc(N)c1-c1ccccc1 ; CCOC(=O)CC(=O)c1ccc(F)cc1 ; Cc1nn2c(O)cc(-c3ccc(F)cc3)nc2c1-c1ccccc1 ; "HOAc"]

Compound 2 (603.7 mg, 3.485 mmol) was dissolved in 70 mL of acetic acid. To this solution 0.94 mL of ethyl 4-fluorobenzoylacetate was added. The mixture was heated to 110° C. under nitrogen for 24 hours. After cooling to room temperature the solvent was evaporated to dryness. The crude product was recrystallized by dissolving in hot ethanol and allowing it to cool. The resulting solid was filtered and the mother liquor was discarded. Compound 3 was obtained in 21% yield.

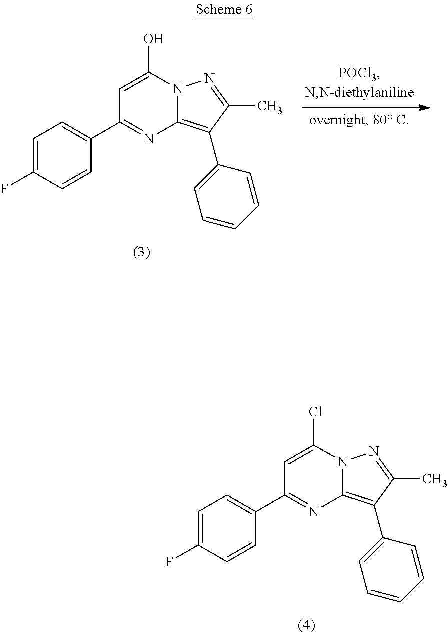 [SMILES: Cc1nn2c(O)cc(-c3ccc(F)cc3)nc2c1-c1ccccc1 ; Cc1nn2c(Cl)cc(-c3ccc(F)cc3)nc2c1-c1ccccc1]

N,N-diethylaniline (0.15 mL, 0.909 mmol) was added to the compound 3 (138.4 mg, 0.433 mmol) in a round bottom flask and placed in an ice bath. Then neat POCl₃ (3 mL) was added dropwise to this mixture at 0° C. and the mixture was stirred at 80 under nitrogen overnight, After cooling to room temperature the mixture was concentrated by evaporating the solvent. The residue was partitioned between ice water and DCM. The pH of the aqueous phase was adjusted to ˜8 with 6N NaOH solution. The layers were separated and the aqueous phase was extracted with DCM. The combine organic layer was dried over magnesium sulfate. The solvent was concentrated under reduced pressure. The crude material was purified using column chromatography on silica using 10% ethyl acetate/hexane gradient to get the compound 4 in a 74% yield.

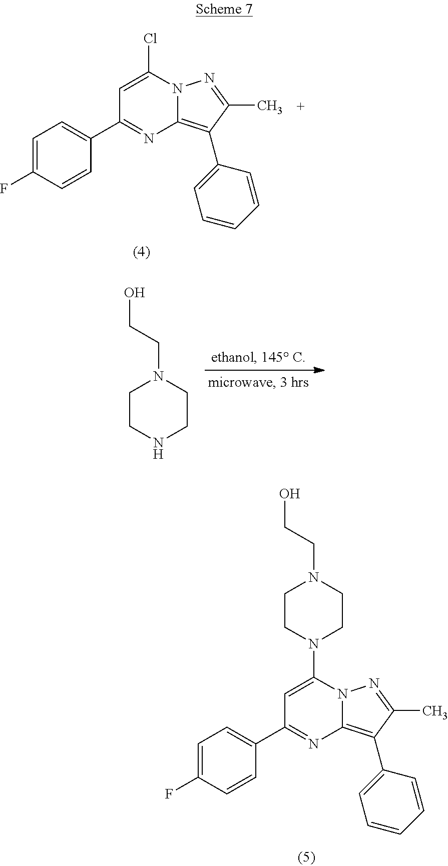 [SMILES: Cc1nn2c(Cl)cc(-c3ccc(F)cc3)nc2c1-c1ccccc1 ; OCCN1CCNCC1 ; Cc1nn2c(N3CCN(CCO)CC3)cc(-c3ccc(F)cc3)nc2c1-c1ccccc1]

To a solution of compound 4 (129.6 mg, 0.384 mmol) in absolute ethanol (3.8 mL) was added piperazine-1-ethanol (0.28 mL, 2.304 mmol) in a microwave reaction vessel. The vessel was sealed and irradiated at 145° C. for 3 hours, After cooling to room temperature the solvent was evaporated and the crude was purified using column chromatography on silica using 5% MeOR/DCM as the solvent to provide the product 5 in 92% yield.

### Example 2: Docking Studies

Docking studies, was performed as described in McCarthy et al, ACS Omega 2019, 4, 2, 292-2930.

Docking studies were performed on a lead compound (C1) that has been shown to bind to GTP-KRAS with high affinity, disrupt effector Rat binding, reduce signal transduction through KRAS, and inhibit proliferation of cancer cells. Furthermore, while this compound does bind to active GTP-KRAS with high affinity, it does not bind to inactive KRAS or GDP- or GTP-bond NRAS and HRAS (two close homologs of KRAS).

### Example 3: Binding Affinity Studies

Measurement of compound binding affinity to KKRAS using microscale thermophoresis (MST). MST is based on the movement of molecules in response to temperature gradient. Measurement was performed following the protocol recommended by the instrument manufacturer (NanoTemper Technologies GmbH) Briefly, the purified KRAS (GDP or GNP-bound) was labeled using the Monolith MT™ Protein Labeling Kit RED-NHS (NanoTemper Tech). KRAS was buffer-exchanged in the labeling buffer (40 mM HEPES, pH 7.5, 5 mM MgCl₂, and 500 mM NaCl) using a spin column (provided in the kit) The concentration of the eluted protein was adjusted (10 to 20 μM) and 2-3 fold concentration of the dye (in labeling buffer) was added (200 μL final volume), The solution was incubated for 30 minutes at room temperature in the dark. Labeled KRAS was purified using the column provided in the kit or MST measurements, 16 point serial dilutions of ligands were made in MST Assay Buffer (40 mM HEPES, pH 7.5, 5 mM MgCl₂, 100 mM NaCl+0.05% Tween-20+2-4% DMSO) and added to 100 nM KRAS. The solutions were loaded in capillaries and measurements were done using 20% LED power and 40% MST power. The data were fit in Igor Pro using Hill equation or the function in the instrument software.

Microscale thermophoresis (MST) assays were used to measure the affinity of lead compound analogs for KRAS. In a typical experiment, 100 nM KRAS labeled with a dye amine-reactive red NT-647 from NanoTemper) was mixed with different concentrations of ligand and incubated in a binding buffer (40 mM HEPES, pH 7.5, 5 mM MgCl₂, 150 mM NaCl, 0.05% polysorbate 20, and 2% DMSO) at room temperature for 10 min before being loaded into a capillary. Thermophoresis is carried out and dissociation constant calculated by monitoring changes in fluorescence intensity. A monolith NT.115 Reader from NanoTemper can be used for MST experiments, with 40% MST and 20% LED power.

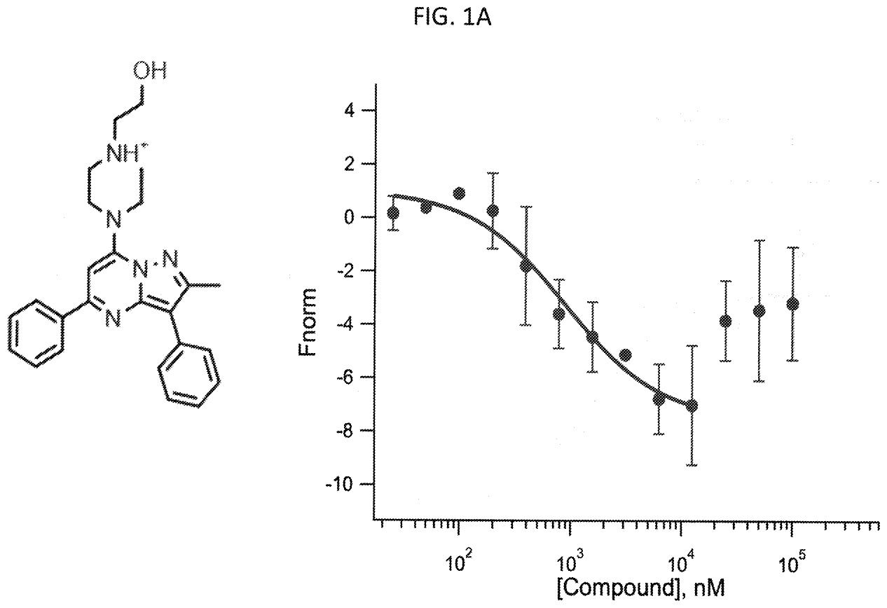

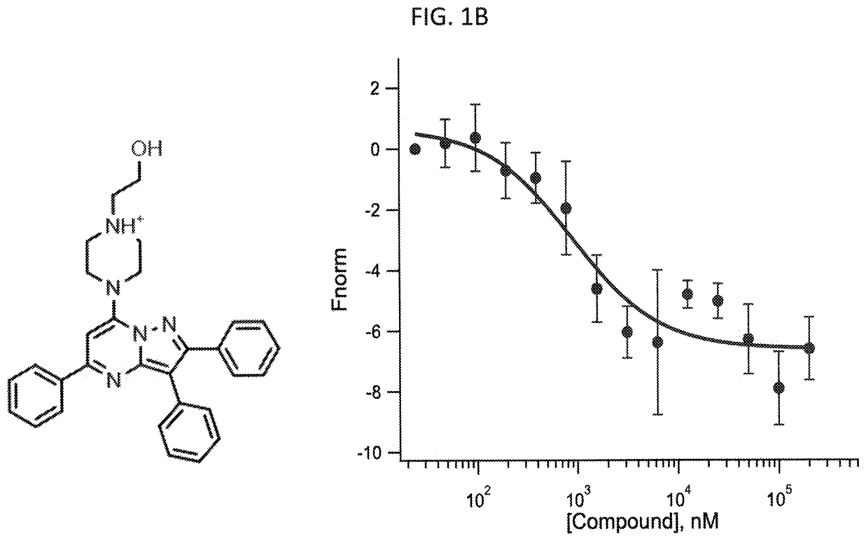

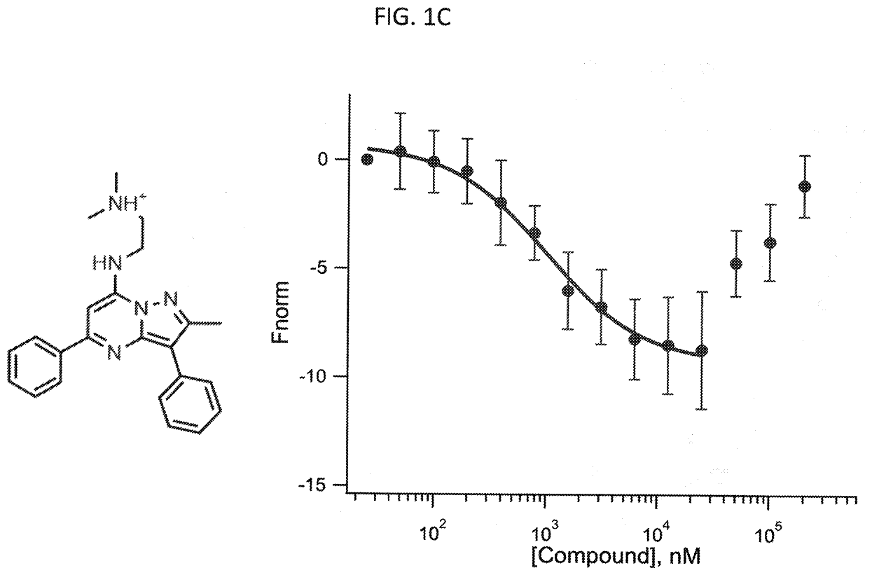

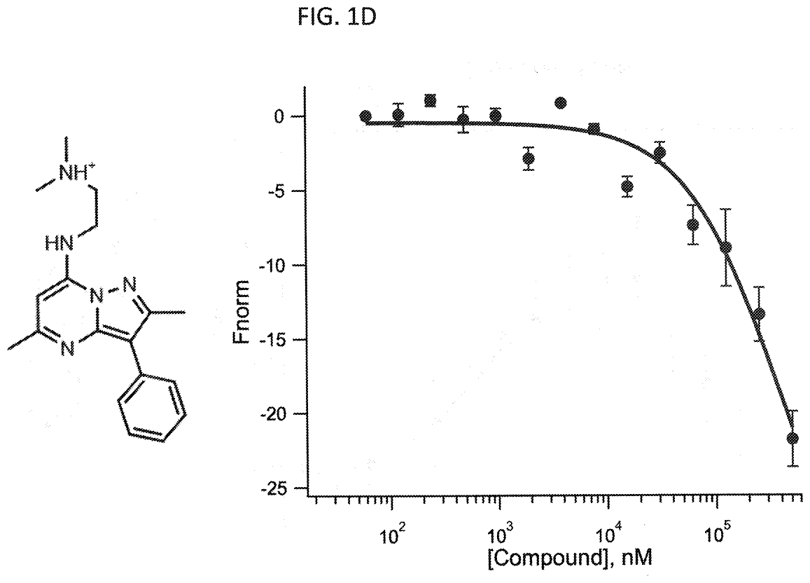

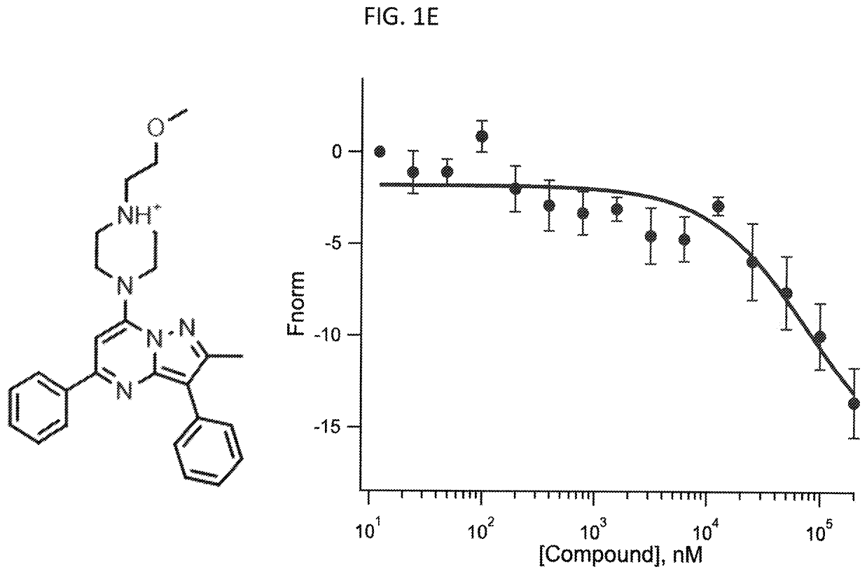

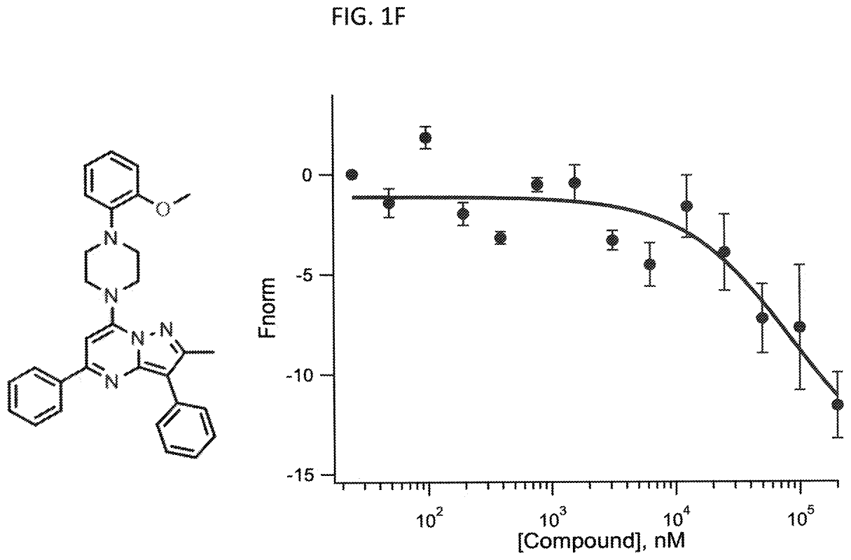

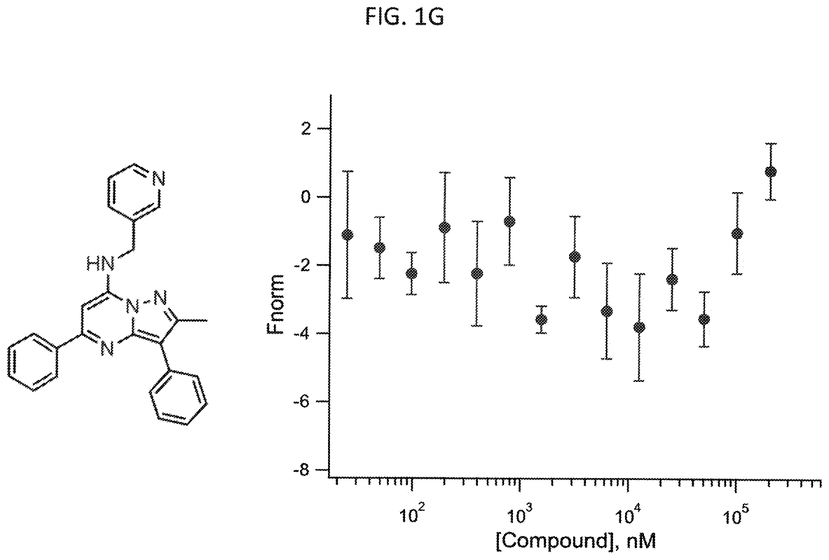

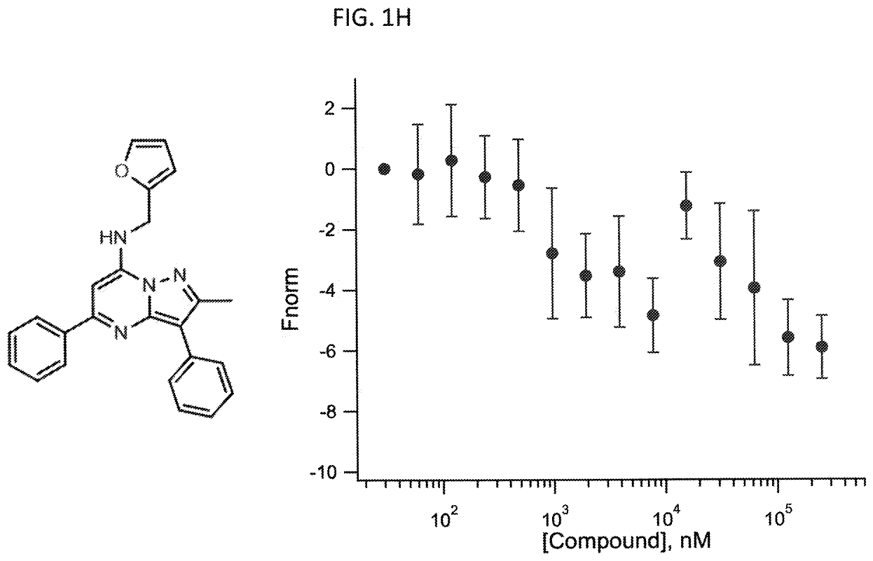

Thermophoresis results for selected compounds are presented in FIGS. 1A-1G. Dissociation constants (IQ)) calculated from the thermophoresis results of selected compounds according to the present disclosure are presented in Table 1 below.

TABLE 1
Dissociation Constants of Selected Compounds.

| Compound Identifier | k_{D} (μM) |
|---|---|
| C1 | 0.87 ± 0.18 |
| C3 | 0.88 ± 0.36 |
| C4 | 0.99 ± 0.14 |
| CS | >200 |
| C6 | >76 |
| C7 | >86 |

In general, a lower k_{D} indicates higher binding affinity. Thus, of the compounds of Table 1, C1, C3, and C4 have the best binding to KRAS of the compounds studied thus far. In some instances (e.g., compounds C5, C6, and C7) the compounds may be poorly soluble, which can interfere with binding. Binding studies using microscale thermophoresis can also be used to characterize interaction of any of the compounds disclosed herein with wild type and or mutant KRAS, including G12V, G12D, G13D, and Q61H mutants, and including both wild type and mutant KRAS bound to GTP or GDP. Instead of or in addition to MST, surface plasmon resonance (SPR) experiments can be used as needed for binding affinity studies.

### Example 4: Biochemical and Cell Biological Characterization

Compounds exhibiting better binding affinity and/or selectivity (e.g., k_{D}≤50 μM) can be evaluated by cell signaling assays to measure levels of phosphorylated RAF (serine/threonine-specific protein kinases), ERK (mitogen-activated protein kinases), AKT (protein kinase B), and ME′ (mitogen-activated protein kinase kinase) in BHK (baby hamster kidney fibroblasts) cells expressing oncogenic mutant KRAS in the presence and absence of a ligand and cell proliferation assays to assess the effect of ligands on growth of cancer cells including panels of pancreatic, colorectal, and lung cancer cells. Western blots can be used to measure levels of total and phosphorylated proteins as indicated. Derivatives that exhibit better efficacy and selectivity in cells can be subjected to additional characterizations to determine mode of action, including fluorescence-based biophysical assays to measure the rates of nucleotide exchange and release, GTP hydrolysis, and effector binding.

CyQuant cell viability assays can be used to quantitatively characterize the effects of disclosed compounds on the growth of a panel of cancer cells, Cell lines useful herein include those listed in Table 2 below.

TABLE 2
Cell Lines.

| Cell Type | Cel1 Line | KRAS Mutation |
|---|---|---|
| Lung cancer | NCI H1975 | Wild type |
| | H522 | Wild type |
| | SKLU-1 | Mutant |
| | NCIH1299 | Mutant |
| | NCIH23 | Mutant |
| Coion cancer | CaCo-2 | Wild type |
| | SK-CO-1 | Mutant |
| | SW948 | Mutant |
| | AQ116 | Mutant |
| Pancreatic cancer | BxPC-3 | Wild type |
| | HPNE | Wild type |
| | MiaPaCa-2 | Mutant |
| | MOH | Mutant |
| | MPano-96 | Mutant |
| | PANC1 | Mutant |
| Endometrial | KLE | Wild type |
| | Ishikawa | Wild type |
| | HedA | Mutant |
| | Hec1B | Mutant |
| | Hec50 | Mutant |
| RASless mouse | MEF | Control |
| embryonic fibroblast | | |

In other experiments, the compounds disclosed herein can be tested for their abilities to affect rates of intrinsic and SOS-catalyzed nucleotide exchange and release. These assays use fluorescently-labeled BODIPY-GDP (BGDP) and BODIPY-GTP (BGTP), which have increased fluorescence intensity when bound to KRAS. Rate of nucleotide exchange is determined by rate of increase in fluorescence after BGTP is added to GDP-KRAS in the absence or presence of SOS Rate of nucleotide release is determined from rate of decrease in, fluorescence when excess GTP is added to KRAS loaded with BGDP in the presence or absence of SOS, where SOS is “Sons of Sevenless” referring to a set of genes encoding guanine nucleotide exchange factors.

In purified systems, fluorescence polarization can be used to determine if compounds disrupt KRAS-effector interaction. This assay is based on the change in fluorescence polarization when GST-RafRBD binds to KRAS loaded with a non-hydrolyzable fluorescent GTP analog (e.g., BODIPY-GTP-γ-S). BODIPY-GTP-γ-S is incubated with or without compound for 30 min at room temperature and increasing concentrations of GST-RaftBD are added. Fluorescence polarization is measured after 30 min and dissociation constant for KRAS-Raf binding is determined by plotting fluorescence polarization versus GST-RafRBD concentration with or without ligand.

To complement fluorescence polarization experiments, compounds will be tested for disruption of KRAS-effector interaction in cell lysates using a pull-down assay, Endogenous GTP-RAS and GFP=GTP-KRAS will be pulled down using GST-RafRBD bound to agarose and detected by Western blot using a pan-RAS antibody, Active GST-GTP-KRAS has a higher molecular weight than endogenous GTP-KRAS.

Effect of ligands on intrinsic and GAP-catalyzed GTP hydrolysis can be tested using the Promega GTPase-Glo Assay kit following a protocol provided by the manufacturer. For selected ligands, rates of GTP hydrolysis can be analyzed using [γ-³²P]GTP assays following published protocols. A commercially available GAP such as NF1-333 or RASA1 can be used for GAP-stimulated hydrolysis.

X-ray crystallographic studies can also be performed to determine structures for ligands that bind to KRAS with high affinity and have significant inhibitory activities in cells as well as exhibiting favorable modes of action including disruption of effector binding, acceleration of GTP hydrolysis, and/or inhibition of SOS activity.

### Example 5

| | | | | EC₅₀ (μM)* | | |
|---|---|---|---|---|---|---|
| | Kd (μM) | | | | SOS | |
| | MST | IF | | Intrinsic | Mediated | |
| Ligand | G12D | WT | G12D | WT | WT | Structure |
| C11″ | 0.87 ± 0.1 | | 4.54 ± 0.1 | | | 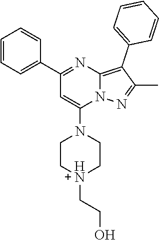 |
| C13″ (**) | | | | 28.8 ± 4.0 | 57.8 ± 4 | 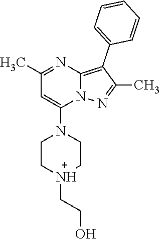 [SMILES: Cc1cc(N2CC[NH+](CCO)CC2)n2nc(C)c(-c3ccccc3)c2n1] |
| C14″ (**) | 0.88 ± 0.4 | 12.3 ± 0.1 | 11.2 ± 0.2 | 12.4 ± 4.8 | there is effect but data is noisy | 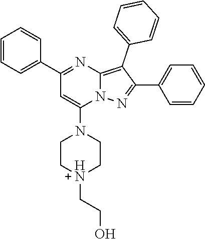 |
| C15 | 0.99 ± 0.1 | | | effect in single- dose @ 25 uM | effect in single- dose @ 25 uM | 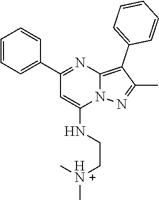 |
| C16 | >200 | | | | effect in single- dose @ 25 uM | 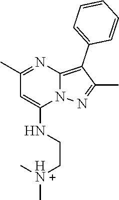 |
| C17″ | >76 | | | effect in single- dose @ 25 uM | effect in single- dose @ 25 uM | 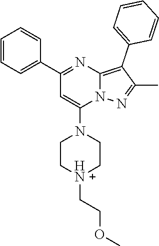 |
| C18 | >86 | | | | | 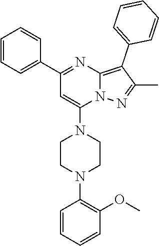 [SMILES: COc1ccccc1N1CCN(c2cc(-c3ccccc3)nc3c(-c4ccccc4)c(C)nn23)CC1] |
| C19 | ND? | | | | | 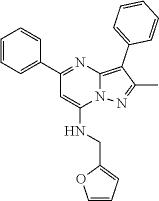 [SMILES: Cc1nn2c(NCc3ccco3)cc(-c3ccccc3)nc2c1-c1ccccc1] |
| C20 | | | | | | 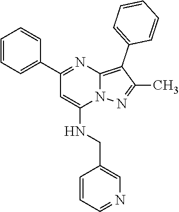 [SMILES: Cc1nn2c(NCc3cccnc3)cc(-c3ccccc3)nc2c1-c1ccccc1] |
| C21″ (**) | 0.52 ± 0.2 | 4.6 ± 0.2 | 6.8 ± 0.5 | 34.0 ± 6.4 | 18.8 ± 3.3 | 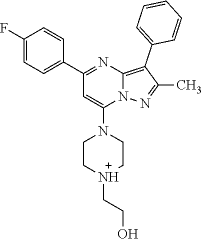 [SMILES: Cc1nn2c(N3CC[NH+](CCO)CC3)cc(-c3ccc(F)cc3)nc2c1-c1ccccc1] |
| C22″ | >100 | | | | no effect in single dose @ 25 uM | 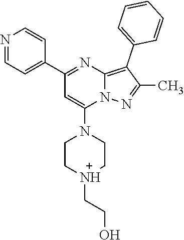 [SMILES: Cc1nn2c(N3CC[NH+](CCO)CC3)cc(-c3ccncc3)nc2c1-c1ccccc1] |
| C23″ | >200 | | | effect in single- dose @ 25 uM | effect in single- dose @ 25 uM | 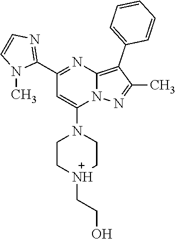 [SMILES: Cc1nn2c(N3CC[NH+](CCO)CC3)cc(-c3nccn3C)nc2c1-c1ccccc1] |
| C24″ | NB | | | | | 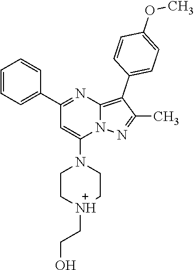 [SMILES: COc1ccc(-c2c(C)nn3c(N4CC[NH+](CCO)CC4)cc(-c4ccccc4)nc23)cc1] |
| C25″ | | | | | effect in single- dose @ 25 uM | 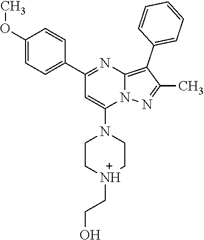 [SMILES: COc1ccc(-c2cc(N3CC[NH+](CCO)CC3)n3nc(C)c(-c4ccccc4)c3n2)cc1] |
| C26″ | | | | effect in single- dose @ 25 uM | effect in single- dose @ 25 uM | 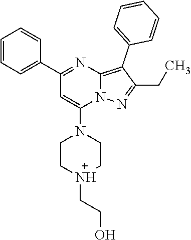 [SMILES: CCc1nn2c(N3CC[NH+](CCO)CC3)cc(-c3ccccc3)nc2c1-c1ccccc1] |
| C27″ | | | | | no effect in single dose @ 25 uM | 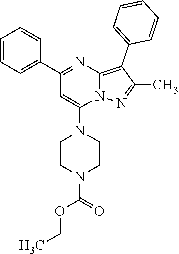 [SMILES: CCOC(=O)N1CCN(c2cc(-c3ccccc3)nc3c(-c4ccccc4)c(C)nn23)CC1] |
| C28″ (**) | | 21.7 ± 0.4 | 23.5 ± 0.8 | effect in single- dose @ 25 uM | No effect in single dose @ 25 uM | 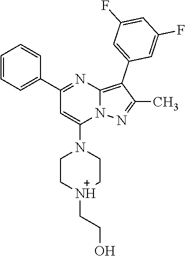 [SMILES: Cc1nn2c(N3CC[NH+](CCO)CC3)cc(-c3ccccc3)nc2c1-c1cc(F)cc(F)c1] |
| C29″ | | | | | effect in single- dose @ 25 uM | 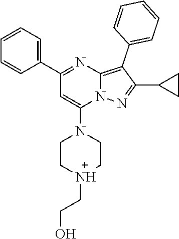 [SMILES: OCC[NH+]1CCN(c2cc(-c3ccccc3)nc3c(-c4ccccc4)c(C4CC4)nn23)CC1] |
| C30″ (**) | | 12.2 ± 0.4 | 18.4 ± 10 | ND; there is effect but data is noisy | 15.9 ± 3.4 | 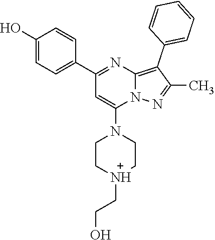 [SMILES: Cc1nn2c(N3CC[NH+](CCO)CC3)cc(-c3ccc(O)cc3)nc2c1-c1ccccc1] |
| C31″ | | | | No effect in single dose @ 25 uM | No effect in single dose @ 25 uM | 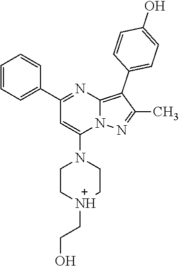 [SMILES: Cc1nn2c(N3CC[NH+](CCO)CC3)cc(-c3ccccc3)nc2c1-c1ccc(O)cc1] |
| C32″ | | | | effect in single- dose @ 25 uM | effect in single- dose @ 25 uM | 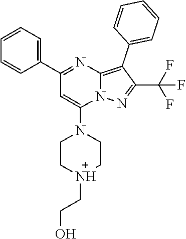 [SMILES: OCC[NH+]1CCN(c2cc(-c3ccccc3)nc3c(-c4ccccc4)c(C(F)(F)F)nn23)CC1] |
| C33″ (**) | | | 79 ± 78 | effect in single- dose @ 25 uM | No effect in single dose @ 25 uM | 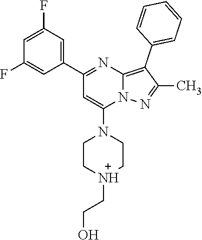 [SMILES: Cc1nn2c(N3CC[NH+](CCO)CC3)cc(-c3cc(F)cc(F)c3)nc2c1-c1ccccc1] |
| C34″ | | | | | | 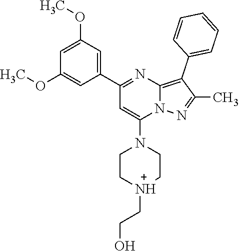 [SMILES: COc1cc(OC)cc(-c2cc(N3CC[NH+](CCO)CC3)n3nc(C)c(-c4ccccc4)c3n2)c1] |
| C35″ | | 8.4 ± 0.2 | 21.6 ± 4 | effect in single- dose @ 25 uM | No effect in single dose @ 25 uM | 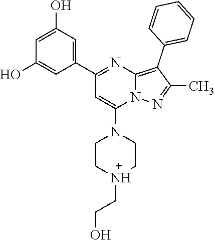 [SMILES: Cc1nn2c(N3CC[NH+](CCO)CC3)cc(-c3cc(O)cc(O)c3)nc2c1-c1ccccc1] |
| C36 | | 20.6 ± 0.4 | 22.4 ± 8 | | | 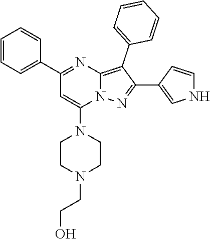 [SMILES: OCCN1CCN(c2cc(-c3ccccc3)nc3c(-c4ccccc4)c(-c4cc[nH]c4)nn23)CC1] |
| C37 | | 22.6 ± 0.3 | 11.8 ± 0.2 | | | 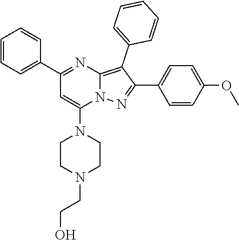 [SMILES: COc1ccc(-c2nn3c(N4CCN(CCO)CC4)cc(-c4ccccc4)nc3c2-c2ccccc2)cc1] |
| C38 | | 19.1 ± 0.1 | 63.1 ± 5.3 | | | 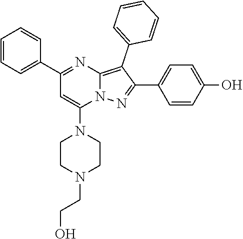 [SMILES: OCCN1CCN(c2cc(-c3ccccc3)nc3c(-c4ccccc4)c(-c4ccc(O)cc4)nn23)CC1] |
| C39 | | 39.5 ± 1.9 | 22.9 ± 2.2 | | | 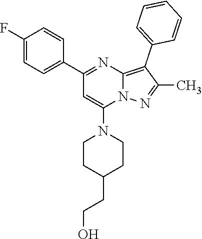 [SMILES: Cc1nn2c(N3CCC(CCO)CC3)cc(-c3ccc(F)cc3)nc2c1-c1ccccc1] |
| C40 | | 63.1 ± 5.3 | 25.7 ± 0.2 | | | 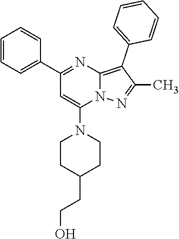 [SMILES: Cc1nn2c(N3CCC(CCO)CC3)cc(-c3ccccc3)nc2c1-c1ccccc1] |
| C41 | | ~20 | | ~15-25 | | 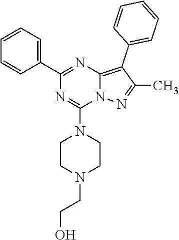 [SMILES: Cc1nn2c(N3CCN(CCO)CC3)nc(-c3ccccc3)nc2c1-c1ccccc1] |
| C42 | | | | | | 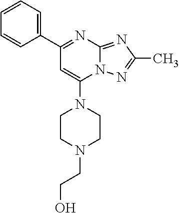 [SMILES: Cc1nc2nc(-c3ccccc3)cc(N3CCN(CCO)CC3)n2n1] |
| C43 | | ~5-15 | | | | 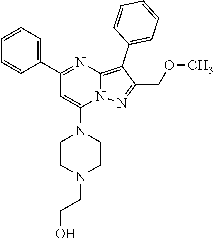 [SMILES: COCc1nn2c(N3CCN(CCO)CC3)cc(-c3ccccc3)nc2c1-c1ccccc1] |
| C44 | | ~5-15 | | | | 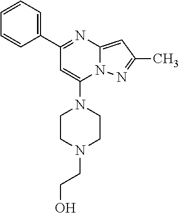 [SMILES: Cc1cc2nc(-c3ccccc3)cc(N3CCN(CCO)CC3)n2n1] |
| C45 | | 28.3 ± 5.4 | | | | 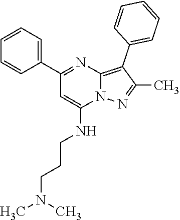 [SMILES: Cc1nn2c(NCCCN(C)C)cc(-c3ccccc3)nc2c1-c1ccccc1] |

*where dose-response measurements have been made, we indicated the EC₅₀ values. In some cases, single-dose tests at 25 microMolar ligand concentration were conducted and the results are indicated with the binary “effect” or “No effect”.
**For these ligands, single-dose intrinsic nucleotide exchange assays have also been done using the G12D mutant KRAS, and the results were positive

### Example 6: General Procedure

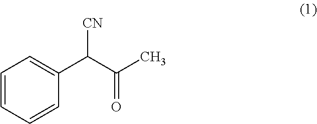

To a 100 mL three neck flask equipped with a reflux condenser and a mechanical stirrer was added 26 mL absolute ethanol and sodium (1.2 g, 52 mmol, 1.3 equiv.). The resulting mixture was stirred at room temperature until the sodium completely dissolved. Phenylacetonitrile (4.8 mL, 40 mmol, 1 equiv.) was added to the sodium ethoxide at room temperature followed by anhydrous ethyl acetate (5.8 mL, 60 mmol, 1.5 equiv.). The resulting mixture was refluxed overnight under nitrogen. After cooling to room temperature the resultant precipitate was dissolved in water and washed with DCM. The organic layer was discarded. Then 1 M HCl was added dropwise until the aqueous phase until any solid precipitate dissolved. The aqueous phase was extracted with DC M and the combined organic phase was washed with saturated NaCl and dried over anhydrous magnesium sulfate. The solvent was concentrated under reduced pressure, 3.398 g of white solid was obtained, 53% yield. The resultant product was used in next step without further purification.

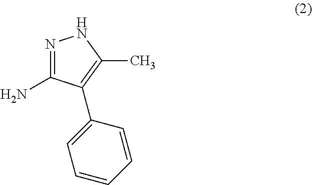

To a solution of compound 1 (697.7 mg, 4.38 mmol, 1 equiv.) in absolute ethanol (23 mL), acetic acid (0.27 mL, 4.73 mmol 1.08 equiv.) was added. While stirring under nitrogen, at room temperature, hydrazine monohydrate (0.43 mL, 8.76 mmol, 2 equiv.) was added dropwise. The reaction mixture was stirred at 60° C. overnight. After cooling to room temperature, the reaction mixture was evaporated to dryness. The residue was extracted with water and saturated NaHCO₃. The organic layer was washed with saturated NaCl and dried over anhydrous magnesium sulfate. The solvent was concentrated under reduced pressure. The crude was purified by column chromatography on silica gel using 10% MeOH in CH₂Cl₂. 665 mg of product was obtained, 88% yield.

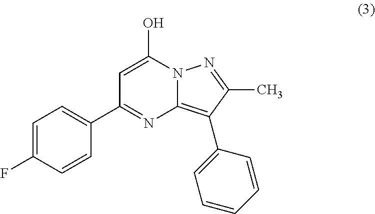 [SMILES: Cc1nn2c(O)cc(-c3ccc(F)cc3)nc2c1-c1ccccc1]

Compound 2 (603.7 mg, 3.49 mmol, 1 equiv.) was dissolved in 70 mL of acetic acid. To this solution ethyl 4-fluorobenzoylacetate (0.94 mL, 5.23 mmol, 1.5 equiv.) was added. The mixture was heated to 110° C. under nitrogen for 24 hours. After cooling to room temperature the solvent was evaporated to dryness. The crude product was recrystallized by dissolving in hot ethanol and allowing it to cool. The resulting solid was filtered and the mother liquor was discarded, affording 234.0 mg of white solid, 21% yield.

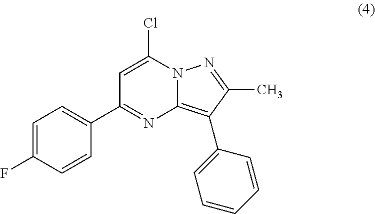 [SMILES: Cc1nn2c(Cl)cc(-c3ccc(F)cc3)nc2c1-c1ccccc1]

N,N-diethylaniline 0.15 mL, 0.91 mmol, 2.1 equiv.) was added to the compound 3 (138.4 mg, 0.43 mmol, 1 equiv.) in a round bottom flask and placed in an ice bath. To this mixture neat POCl₃ (3 mL) was added dropwise at 0° C. The reaction was stirred at 80° C. under nitrogen overnight. After cooling to room temperature the mixture was concentrated by evaporating the solvent. The residue was partitioned between ice water and DC M. The pH of the aqueous phase was adjusted to ˜8 with 6N NaOH solution. The layers were separated and the aqueous phase was extracted with DCM. The organic phases were combined and washed with brine, dried over anhydrous MgSO₄, filtered and concentrated to yield the crude product. The crude was purified by column chromatography on silica gel using 10% EtOAc in hexane, 109 mg of product was obtained, 75% yield.

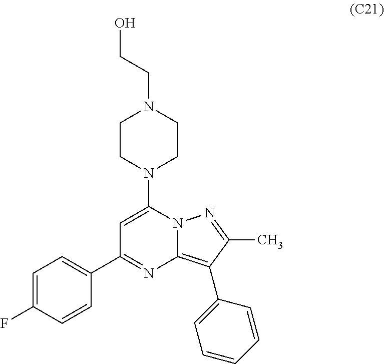 [SMILES: Cc1nn2c(N3CCN(CCO)CC3)cc(-c3ccc(F)cc3)nc2c1-c1ccccc1]

To a solution of compound 4 (129.6 mg, 0.38 mmol, 1 equiv. in absolute ethanol (3.8 mL) was added piperazine-1 ethanol (0.28 mL, 2.30 mmol, 6 equiv.) in a microwave reaction vessel. The vessel was sealed and irradiated at 145° C. for 3 hours. After cooling to room temperature the solvent was evaporated and the crude was purified by column chromatography on silica gel using 5% MeOH in CH₂Cl₂, 152 mg of product was obtained, 92% yield.

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

### Example 7

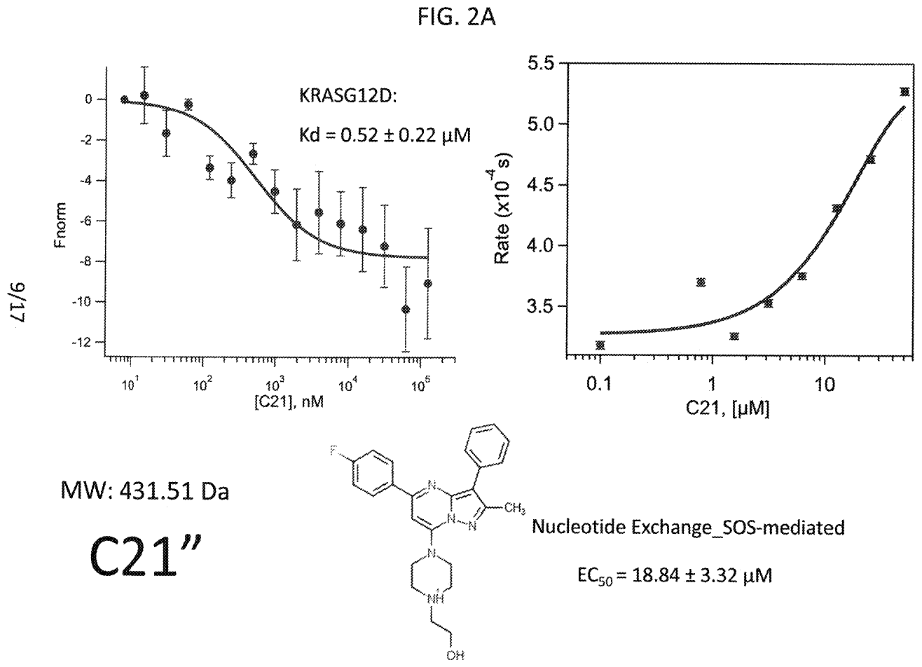

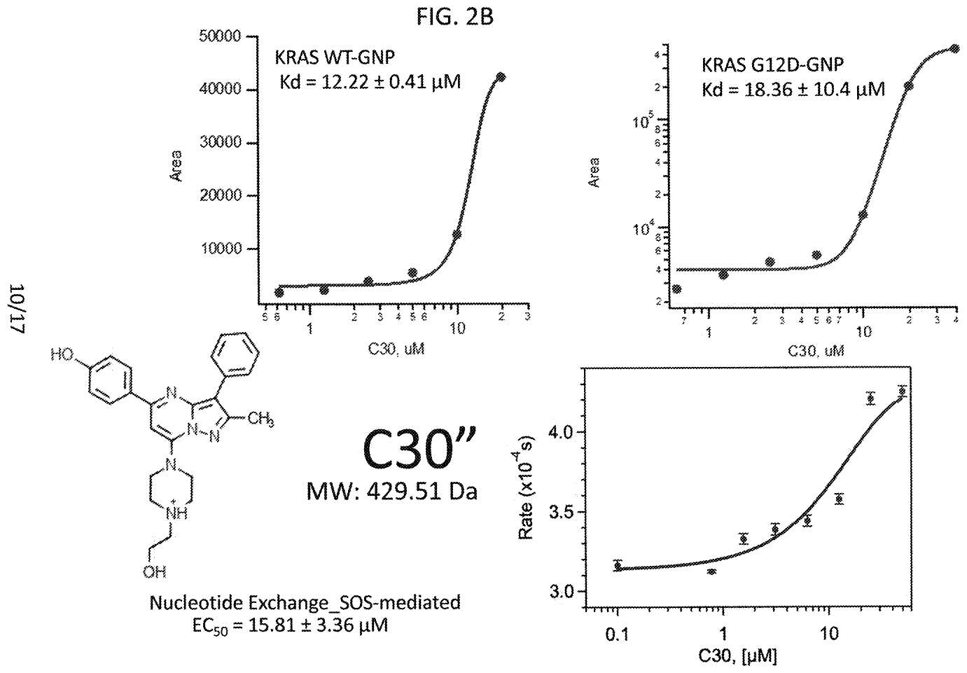

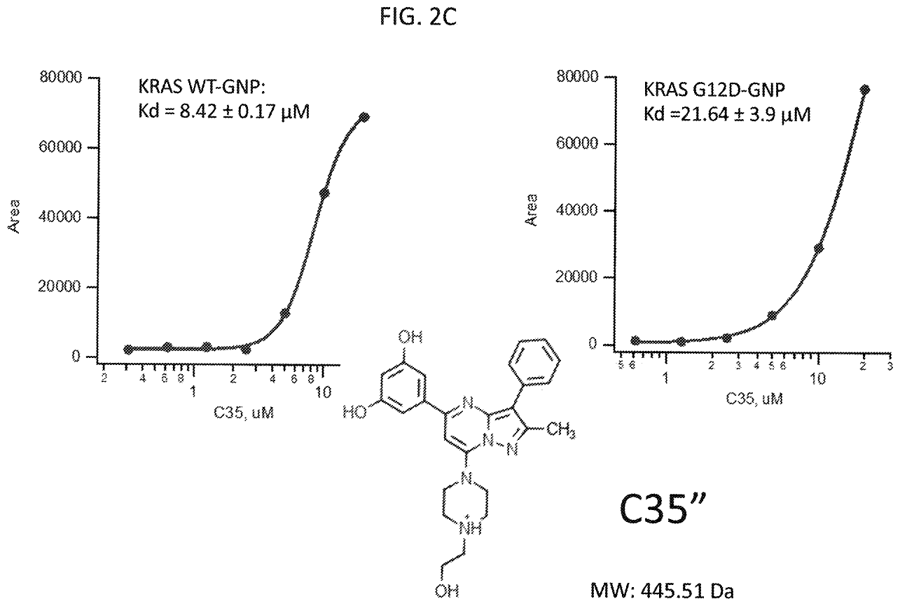

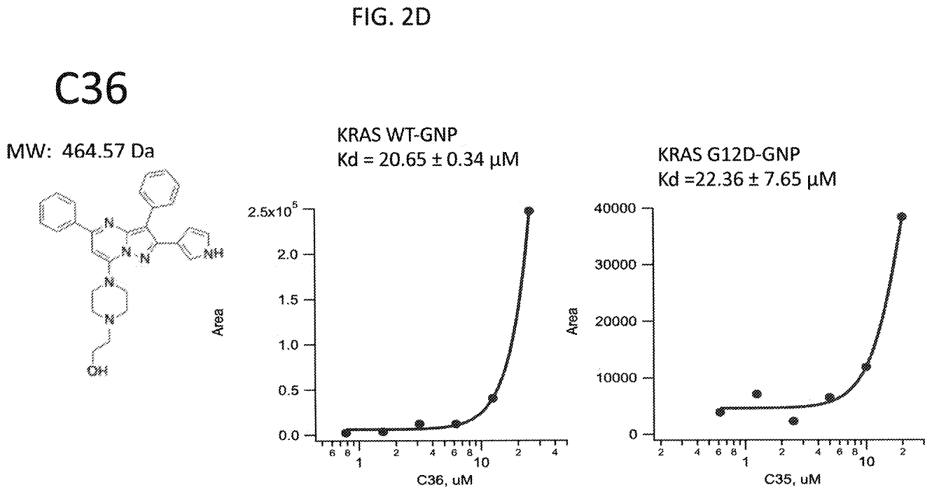

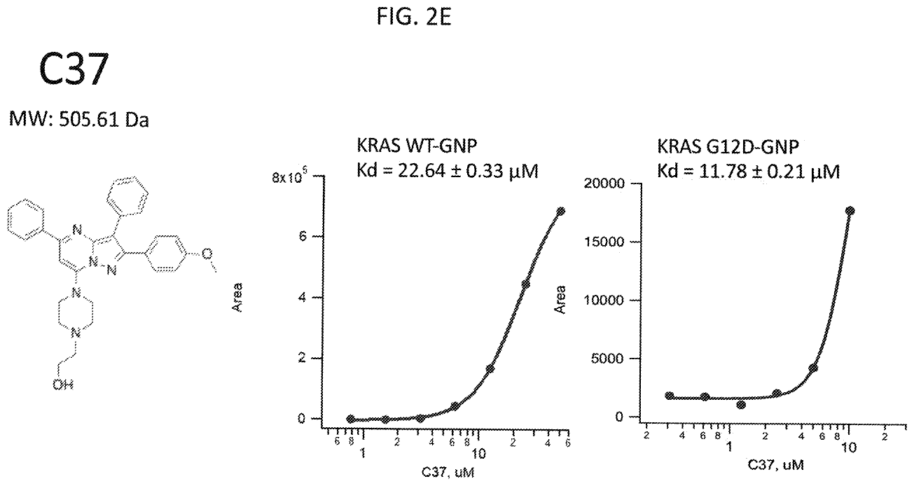

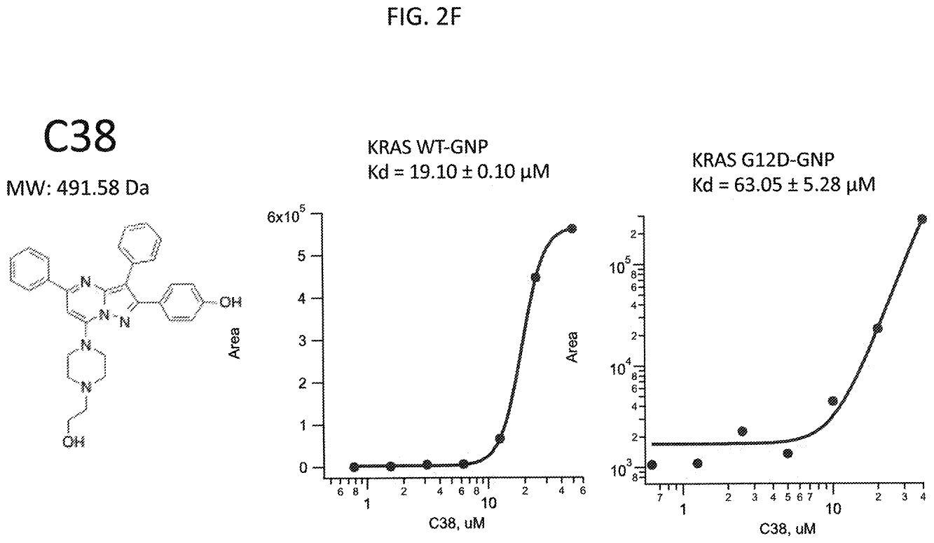

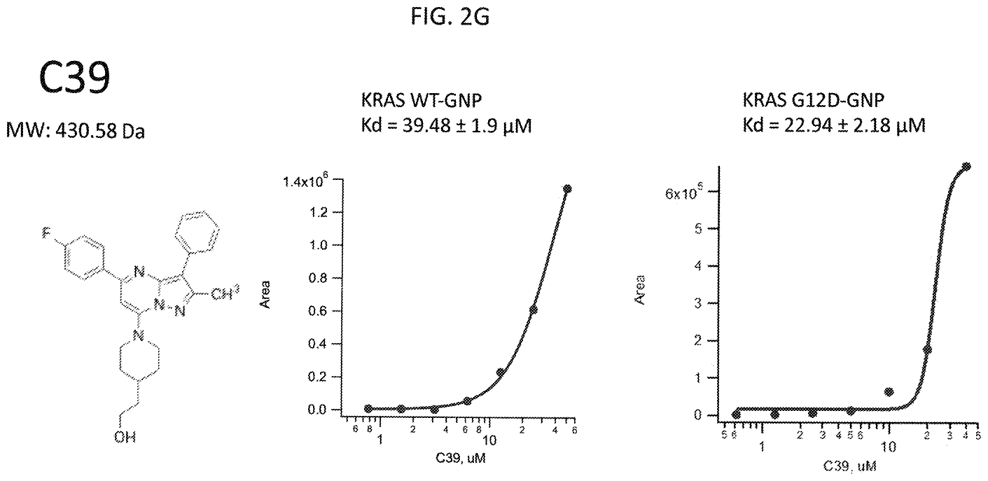

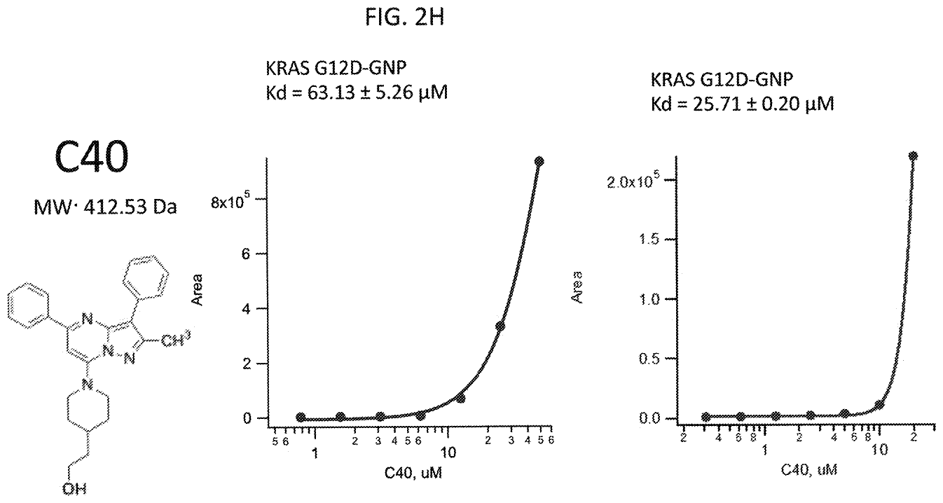

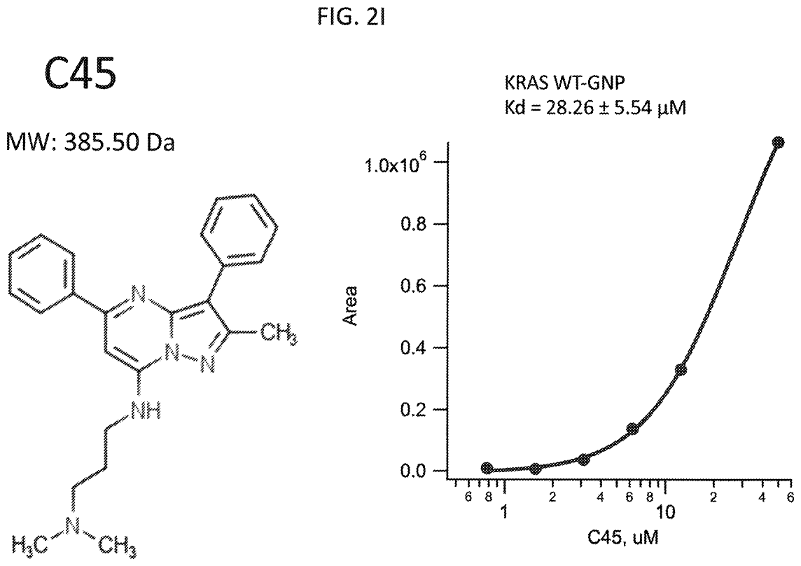

FIGS. 2A-I illustrates the measurements of binding affinity of ligands to wild type (WT) or G12D KRAS bound to the GTP analogue GNP and, for selected exemplary embodiments, effect of ligands on the rate of SOS-mediated nucleotide exchange reaction, Binding affinity was determined using intrinsic fluorescence, which made use of the ligands' intrinsic fluorescence and the accompanying change (decrease or increase in intensity) upon binding to KRAS.

Solutions of ligand (0 to 50 μM) are mixed with equal volumes of KRAS (fixed concentration) and fluorescence spectra are obtained. The difference spectra (between KRAS-ligand mixture and ligand alone) are plotted and the area under the curve is obtained. Kd is estimated from the plot of area versus the ligand concentration. SOS-mediated nucleotide rate was monitored using the fluorescence intensity increase of BODIPY-GTP (B GTP) as it displaces GDP from KRAS. The reaction mixture (100 μL, 96 well plate) contained 0.5 μM K-Ras-GDP, 0.5 μm B-GTP, 0.5 μM SOS and varying concentrations of ligands (0 to 25 μM). B-GTP and SOS mixture was added just before measurements(excitation: 485 nm, emission: 510 nm; TECAN Infinite 200 plate reader). The reaction was monitored for 1 hour. The experiments were conducted with minimal light, Fluorescence intensities were normalized with respect to the value obtained at 120 s and the traces were fit with exponential function (Igor Pro, Wavemetrics).

Note: For FIGS. 1A-H 1 (as well as one of the curves in FIGS. 2A-I), dissociation constant (Kd) was determined using either Microscale Thermophoresis (MST) or Intrinsic Fluorescence (IF) assay. MST assay uses fluorescence to monitor movement of molecules in response to temperature gradient created by infrared laser. Purified KRAS was labeled with the Monolith MTTM Protein Labeling Kit RED-NHS (NanoTemper) and assayed following vendor's protocol.

### LIST OF EMBODIMENTS

1. A compound having a structure represented by a formula;

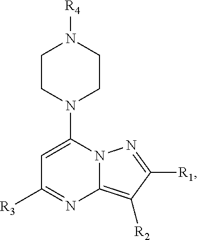

wherein R₁ is aryl, substituted aryl, —(C1-C7 alkanediyl—R₅, or —(C1-C7 alkanediyl)-(C═O)—R₅;
wherein each of R₂ and R₃ is independently C1-C8 alkyl, cycloalkyl heterocycloalkyl, aryl, heteroaryl, substituted aryl, or substituted heteroaryl;
wherein R₄ is —(C1-C7 alkyl) or —(C1-C7 alkanediyl)-R₆; and
wherein each of R₅ and R₆ is independently hydrogen, methoxy, —SF₅, —OH, —SH, —NH₂, or halogen;
or a pharmaceutically acceptable salt, solvate, or ester, thereof;
provided that the compound does not have a structure represented by a formula of:

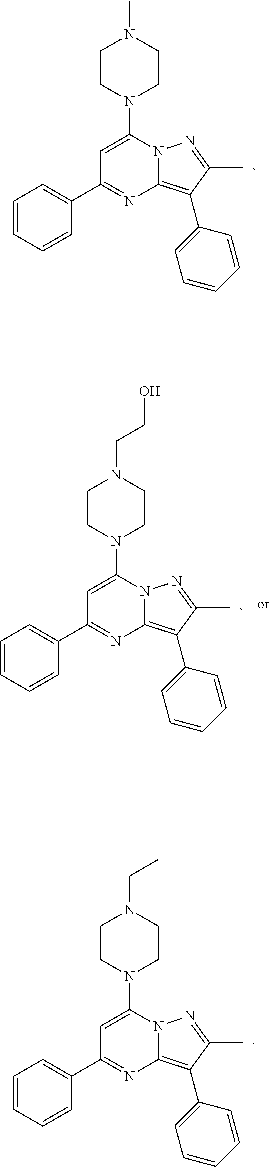 [SMILES: Cc1nn2c(N3CCN(C)CC3)cc(-c3ccccc3)nc2c1-c1ccccc1 ; Cc1nn2c(N3CCN(CCO)CC3)cc(-c3ccccc3)nc2c1-c1ccccc1 ; CCN1CCN(c2cc(-c3ccccc3)nc3c(-c4ccccc4)c(C)nn23)CC1]

2. The compound of embodiment 1, wherein R₁ is methyl, —CF₃ or phenyl.

3. The compound of embodiment 1, wherein R₂ is phenyl or 4-methoxyphenyl.

4. The compound of embodiment 1, wherein R₃, is phenyl, methyl, 4-methoxyphenyl, 4-pyridinyl, N-methylimidazole, hydroxyphenyl (preferably 4-hydroxyphenyl) or 4-fluorophenyl.

5. The compound of embodiment 1, wherein R₄ is —(CH₂)₂OH, —(CH₂)₂—OCH₃ or 2-methoxyphenyl.

6. The compound of embodiment 1, having a structure represented by a formula:

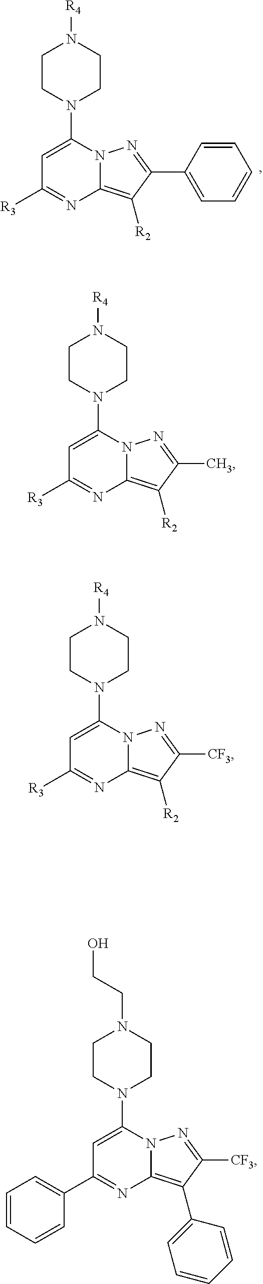

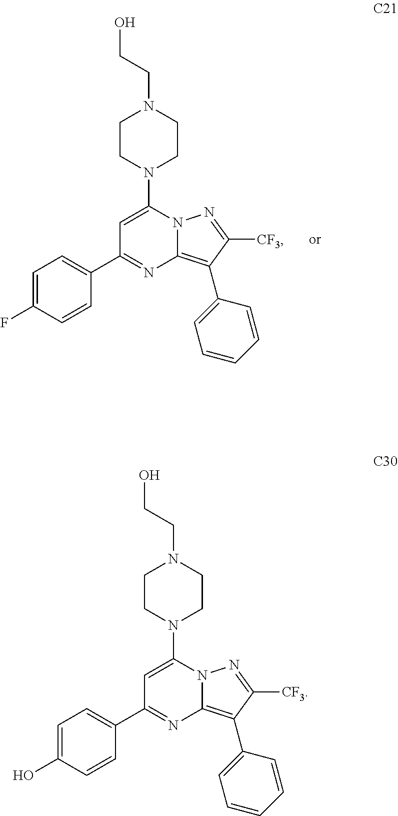 [SMILES: OCCN1CCN(c2cc(-c3ccc(F)cc3)nc3c(-c4ccccc4)c(C(F)(F)F)nn23)CC1 ; OCCN1CCN(c2cc(-c3ccc(O)cc3)nc3c(-c4ccccc4)c(C(F)(F)F)nn23)CC1]

7, The compound of embodiment 1, having a structure represented by a formula:

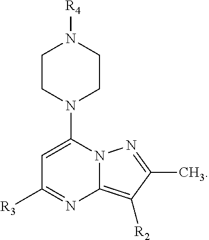

8. The compound of embodiment 1, having a structure represented by a formula:

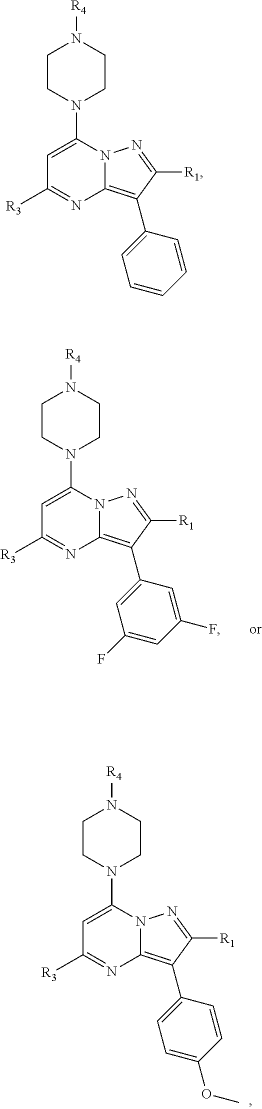

9. The compound of embodiment 1, having a structure represented by a formula:

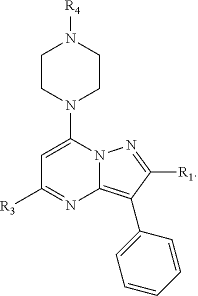

10, The compound of embodiment 1, having a structure represented by a formula:

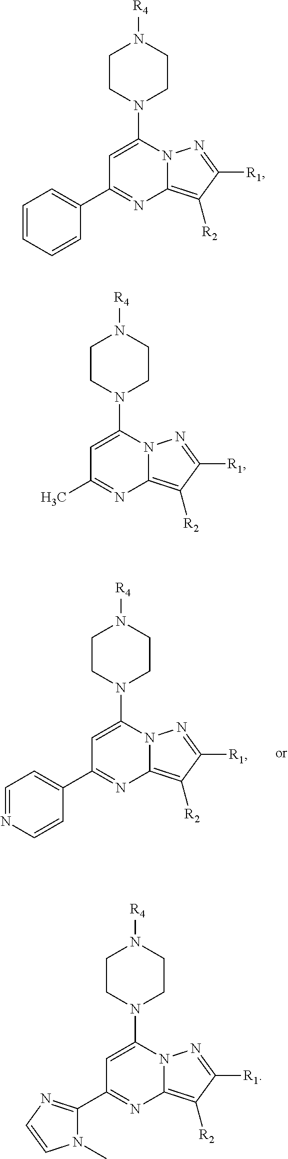

11. The compound of embodiment 1, having a structure represented by a formula:

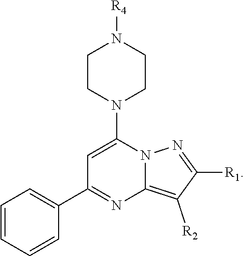

12. The compound of embodiment 1, having a structure represented by a formula:

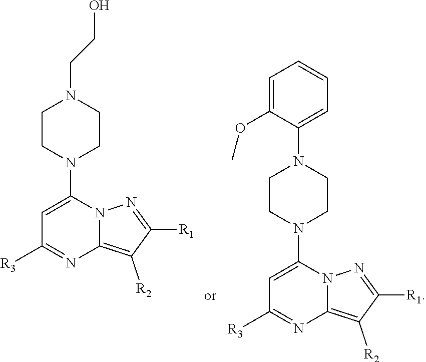

13. The compound of embodiment 1, having a structure represented by formula:

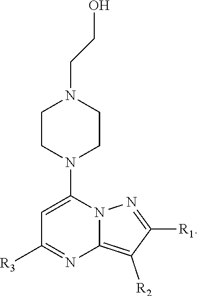

14. The compound of err embodiment 1, having a structure represented by a formula:

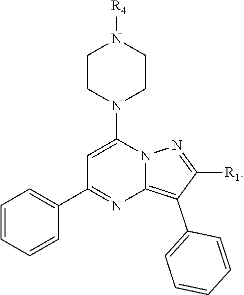

15. The compound of embodiment Error! Reference source not found., wherein R₁ is methyl, —CF₃, or phenyl.

16. The compound of embodiment Error! Reference source not found., wherein R₄ is—(CH₂)₂OH, —(CH₂)₂—OCH₃, or 2-methoxyphenyl.

17. The compound of embodiment 1, having a structure represented by a formula:

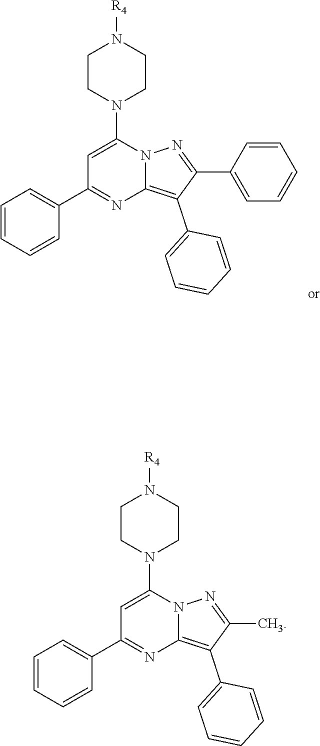

18. The compound of embodiment 1, having a structure represented by a formula:

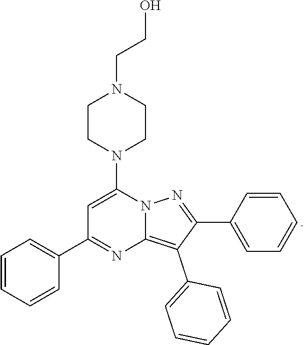 [SMILES: OCCN1CCN(c2cc(-c3ccccc3)nc3c(-c4ccccc4)c(-c4ccccc4)nn23)CC1]

19, The compound of embodiment 1, having a structure represented by a formula:

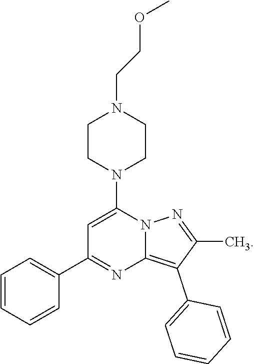 [SMILES: COCCN1CCN(c2cc(-c3ccccc3)nc3c(-c4ccccc4)c(C)nn23)CC1]

20. The compound of embodiment 1, having a structure represented by a formula:

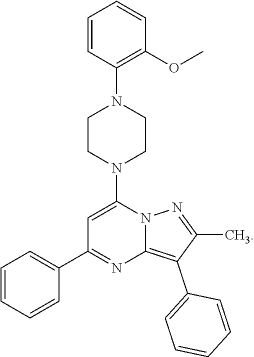 [SMILES: COc1ccccc1N1CCN(c2cc(-c3ccccc3)nc3c(-c4ccccc4)c(C)nn23)CC1]

21. A compound having a structure represented by a formula:

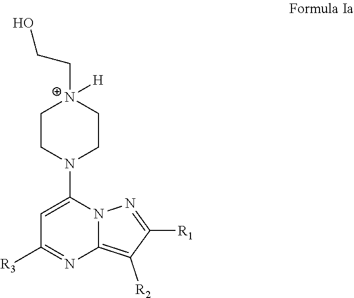

wherein R₁ is aryl, substituted aryl, —(C1-C7 alkanediyl)-R₅, or —(C1-C7 alkanediyl)-(C═O)—R₅;
wherein each of R₂ and R₃ is independently C1-C8 alkyl, cycloalkyl, heterocycloalkyl, aryl, hetero aryl, substituted aryl, or substituted heteroaryl; wherein R₄ is -1C1-C7 alkyl) or —(C1-C7 alkanediyl)-R₅; and wherein each of R₅ and R is independently hydrogen, methoxy, —SF₅, OH, —SH, —NH₂, or halogen; or a pharmaceutically acceptable salt, solvate, or ester thereof.

22. The compound of embodiment 21, wherein said compound is any of:

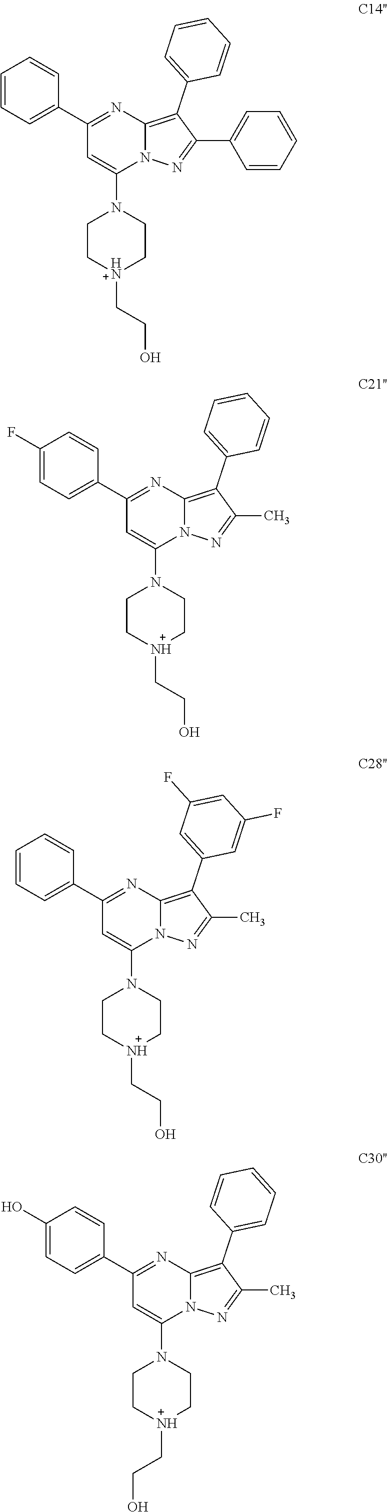

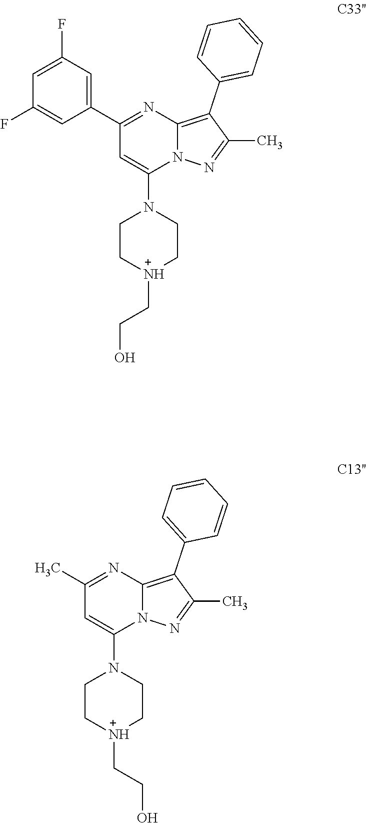 [SMILES: Cc1nn2c(N3CC[NH+](CCO)CC3)cc(-c3cc(F)cc(F)c3)nc2c1-c1ccccc1 ; Cc1cc(N2CC[NH+](CCO)CC2)n2nc(C)c(-c3ccccc3)c2n1]

23. A Pharmaceutical composition comprising a therapeutically effective amount of a compound of any of embodiments 1-Error! Reference source not found., or a pharmaceutically acceptable salt, solvate, and a pharmaceutically acceptable carrier.

24, A method for the treatment of a cancer associated with KRAS dysfunction in a mammal comprising the step of administering to the mammal a therapeutically effective amount of at least one compound of any of embodiments 1-Error! Reference source not found., or a pharmaceutically acceptable salt thereof.

25. The method of embodiment 9, wherein the mammal is a human.

26. The method of embodiment 9, wherein the mammal has been diagnosed with a need for treatment of the cancer prior to the administering step.

27. The method of embodiment 9, further comprising the step of identifying a mammal in need of treatment of the cancer.

28. The method of embodiment 9, wherein cancer is selected from colorectal cancer, small cell lung cancer, non-small cell lung cancer, pancreatic cancer, ovarian cancer, thyroid cancer, stomach cancer, squamous cell carcinoma, melanoma, bladder cancer, renal cell carcinoma, another cancer, or a combination thereof.

29. A method for the treatment of a disorder of uncontrolled cellular proliferation associated with KRAS dysfunction in a mammal comprising the step of administering to the mammal a therapeutically effective amount of at least one compound of any of embodiments 1-Error! Reference source not found., or a pharmaceutically acceptable salt thereof.

30. The method of embodiment 11, wherein the mammal is a human.

31. The method of embodiment 29, wherein the mammal has been diagnosed with a need for treatment of the disorder prior to the administering step.

32. The method of embodiment 11, further comprising the step of identifying a mammal in need of treatment of the disorder.

33. A method for inhibition of aberrant KRAS activity in a mammal comprising the step of administering to the mammal a therapeutically effective amount of at least one compound of any of embodiments 1-Error! Reference source not found., or a pharmaceutically acceptable salt thereof.

34. The method of embodiment 12, wherein the mammal is a human.

35, The method of embodiment 12, wherein the mama al has been diagnosed with a need for inhibition of aberrant KRAS activity prior to the administering step.

36. The method of embodiment 12, further comprising the step of identifying a mammal in need for inhibition of aberrant KRAS activity.

37. A method for inhibition of aberrant KRAS activity in at least one cell, comprising the step of contacting the at least one cell with an effective, amount of at least one compound of any of embodiments 1-Error! Reference source not found., or a pharmaceutically acceptable salt thereof.

38. The method of embodiment 13, wherein the cell is mammalian.

39. The method of embodiment 37, wherein the cell is human.

40. The method of embodiment 37, wherein the cell has been isolated from a mammal prior to the contacting step.

41. The method of embodiment 37, wherein contacting is via administration to a mammal.

42. The method of embodiment 41, wherein the mammal has been diagnosed with a need for inhibition of aberrant KRAS activity prior to the administering step.

43. The method of embodiment 41, wherein the mammal has been diagnosed with a need for treatment of a disorder related to aberrant KRAS activity prior to the administering step.

44. A kit comprising at least one compound of any of embodiments 1-Error! Reference source not found., or a pharmaceutically acceptable salt thereof; and one or more of:
a) at least one agent known to decrease aberrant KRAS activity;
b) at least one agent known to treat a cancer associated with aberrant KRAS activity;
c) at least one agent known to treat a disease of uncontrolled cellular proliferation; and
d) instructions for treating a disorder associated with KRAS dysfunction.

45. The kit of embodiment 44, wherein the at least one cot pound and the at least one agent are co-formulated.

46. The kit of embodiment 44, wherein the at least one compound and the at least one agent are co-packaged.

1. A compound having a structure represented by a formula: 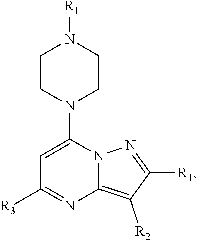
wherein R₁ is aryl, substituted aryl, —(C1-C7 alkanediyl)-R₅, or —(C1-C7 alkanediyl)-(C═O)—R₅;
wherein each of R₂ and R₃ is independently C1-C8 alkyl, cycloalkyl, heterocycloalkyl, aryl, heteroaryl, substituted aryl, or substituted heteroaryl;
wherein R₄ is 2-methoxyphenyl, —(C1-C7 alkyl) or—(C1-C7 alkanediyl)-R₆; and
wherein each of R₅ and R₆ is independently hydrogen,-methoxy, —SF₅, —OH, —SH, —NH₂, or halogen;
or a pharmaceutically acceptable salt, solvate, or ester, thereof;
provided that the compound does not have a structure represented by a formula of:

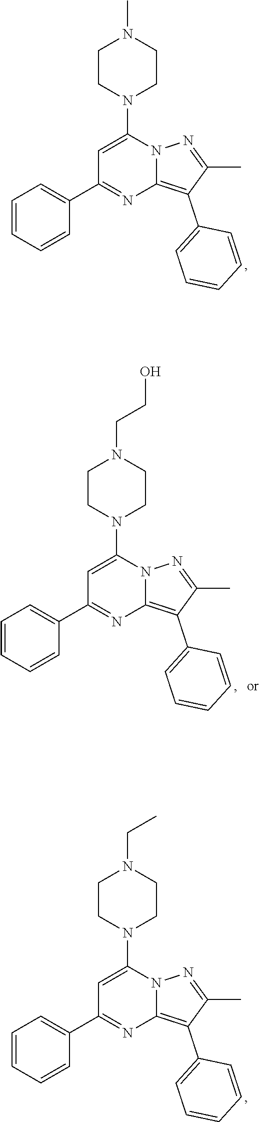 [SMILES: Cc1nn2c(N3CCN(C)CC3)cc(-c3ccccc3)nc2c1-c1ccccc1 ; Cc1nn2c(N3CCN(CCO)CC3)cc(-c3ccccc3)nc2c1-c1ccccc1 ; CCN1CCN(c2cc(-c3ccccc3)nc3c(-c4ccccc4)c(C)nn23)CC1]

2. The compound of claim 1, wherein R₁ is methyl, —CF₃ or phenyl.

3. The compound of claim 1, wherein R₃ is phenyl, methyl, 4-methoxyphenyl, 4-pyridinyl, N-methylimidazole, hydroxyphenyl or 4-fluorophenyl.

4. The compound of claim 1, wherein R₄ is —(CH₂)₂—OH, —(CH₂)₂—OCH₃, or 2-methoxyphenyl.

5. The compound of claim 1, having a structure represented by a formula: 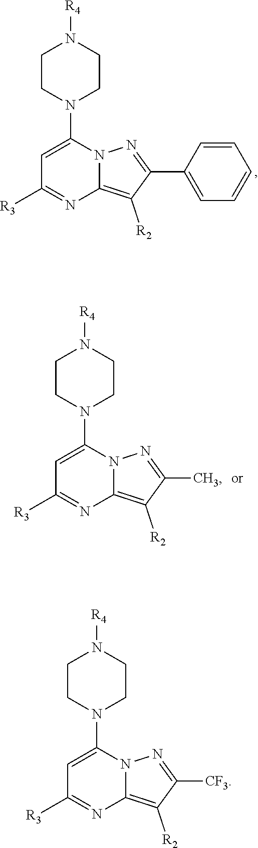
or

6. A compound having a structure represented by a formula: 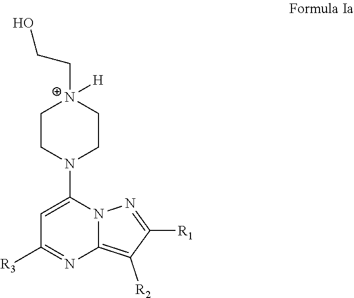
wherein R₁ is aryl, substituted aryl, —(C1-C7 alkanediyl)-R₅, or —(C1-C7 alkanediyl)-(C═O)—R₅;
wherein each of R₂ and R₃ is independently C1-C8 alkyl, cycloalkyl, heterocycloalkyl, aryl, heteroaryl, substituted aryl, or substituted heteroaryl;
and
wherein each of R₅ is independently hydrogen, methoxy, —SF₅, —OH, —SH, —NH₂, or halogen; or a pharmaceutically acceptable salt, solvate, or ester thereof.

7. The compound of claim 6, wherein said compound is any of:

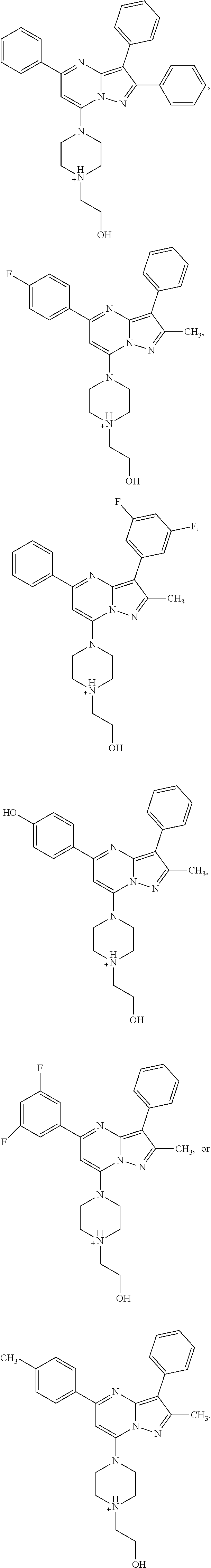

8. A pharmaceutical composition comprising a therapeutically effective amount of a compound of claim 1, or a pharmaceutically acceptable salt or solvate thereof, and a pharmaceutically acceptable carrier.

9. A method for the treatment of a cancer associated with KRAS dysfunction in a mammal comprising the step of administering to the mammal a therapeutically effective amount of at least one compound of claim 1, or a pharmaceutically acceptable salt thereof.

10. The method of claim 9, wherein the cancer is selected from colorectal cancer, small cell lung cancer, non-small cell lung cancer, pancreatic cancer, ovarian cancer, thyroid cancer, stomach cancer, squamous cell carcinoma, melanoma, bladder cancer, renal cell carcinoma, or a combination thereof.

11. A method for the treatment of a disorder of uncontrolled cellular proliferation associated with KRAS dysfunction in a mammal comprising the step of administering to the mammal a therapeutically effective amount of at least one compound of claim 1, or a pharmaceutically acceptable salt thereof.

12. A method for inhibition of aberrant KRAS activity in a mammal comprising the step of administering to the mammal a therapeutically effective amount of at least one compound of claim 1, or a pharmaceutically acceptable salt thereof.

13. A method for inhibition of aberrant KRAS activity in at least one cell, comprising the step of contacting the at least one cell with an effective amount of at least one compound of claim 1, or a pharmaceutically acceptable salt thereof.

14. A kit comprising at least one compound of claim 1, or a pharmaceutically acceptable salt thereof; and one or more of:
a) at least one agent known to decrease aberrant KRAS activity;
b) at least one agent known to treat a cancer associated with aberrant KRAS activity;
c) at least one agent known to treat a disease of uncontrolled cellular proliferation; and
d) instructions for treating a disorder associated with KRAS dysfunction.

15. The kit of claim 14, wherein the at least one compound and the at least one agent are co-formulated.

16. The compound of claim 1, wherein said compound is represented by a formula chosen from: 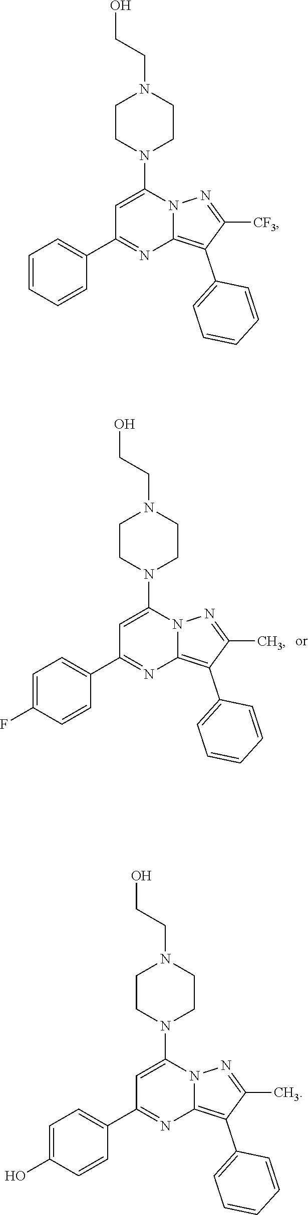 [SMILES: OCCN1CCN(c2cc(-c3ccccc3)nc3c(-c4ccccc4)c(C(F)(F)F)nn23)CC1 ; Cc1nn2c(N3CCN(CCO)CC3)cc(-c3ccc(F)cc3)nc2c1-c1ccccc1 ; "or" ; Cc1nn2c(N3CCN(CCO)CC3)cc(-c3ccc(O)cc3)nc2c1-c1ccccc1]
